(19) United States
(12) Patent Application Publication
(10) Pub. No.: US 2024/0424143 A1
(43) Pub. Date: Dec. 26, 2024

(54) ENGINEERED MUSCLE AND CENTRAL NERVOUS SYSTEM COMPOSITIONS

(71) Applicants: The Broad Institute, Inc., Cambridge, MA (US); President and Fellows of Harvard College, Cambridge, MA (US); Massachusetts Institute of Technology, Cambridge, MA (US)

(72) Inventors: Pardis Sabeti, Cambridge, MA (US); Mohammadsharif Tabebordbar, Cambridge, MA (US); Simon Ye, Cambridge, MA (US)

(21) Appl. No.: 18/689,897

(22) PCT Filed: Sep. 8, 2022
(86) PCT No.: PCT/US22/76117

§ 371 Date: Mar. 7, 2024

Related U.S. Application Data
(60) Provisional application No. 63/242,008, filed on Sep. 8, 2021.

(51) Int. Cl. A61K 48/00; C12N 15/86

(52) U.S. Cl. CPC A61K 48/0058; C12N 15/86; C12N 2750/14122; C12N 2750/14143; C12N 2750/14152

(57) ABSTRACT

Described in several exemplary embodiments are compositions including a targeting moiety effective to target a central nervous system cell and formulations thereof. In certain embodiments, the targeting moiety is composed of one or more n-mer inserts, that can include one or more RGD motifs, and/or one or more P-motifs. Also described in certain example embodiments are vector systems configured to generate polypeptides containing the one or more targeting moieties. Also described herein are methods of generating a targeting moiety effective to target a central nervous system cell and using the compositions containing the targeting moieties described herein, such as to deliver a cargo to a subject and/or treat a central nervous system disease, disorder, or system thereof.

### CROSS-REFERENCE TO RELATED APPLICATIONS

This application claims the benefit of and priority to U.S. Provisional Patent Application No. 63/242,008, filed on Sep. 8, 2021, the contents of which is incorporated by reference herein in its entirety.

### REFERENCE TO AN ELECTRONIC SEQUENCE LISTING

The contents of the electronic sequence listing (“BROD-5455WP_ST26.xml”; Size is 359,525 bytes and it was created on Sep. 7, 2022) is herein incorporated by reference in its entirety.

### TECHNICAL FIELD

The subject matter disclosed herein is generally directed to engineered central nervous system targeting compositions including, but not limited to, recombinant adeno-associated virus (AAV) vectors, and systems, compositions, and uses thereof.

### BACKGROUND

Recombinant AAVs (rAAVs) are the most commonly used delivery vehicles for gene therapy and gene editing. Nonetheless, rAAVs that contain natural capsid variants have limited cell tropism. Indeed, rAAVs used today mainly infect the liver after systemic delivery. Further, the transduction efficiency of conventional rAAVs in other cell-types, tissues, and organs by these conventional rAAVs with natural capsid variants is limited. Therefore, AAV-mediated polynucleotide delivery for diseased that affect cells, tissues, and organs other than the liver, such as the central nervous system) typically requires an injection of a large dose of virus (typically about 2×10¹⁴ vg/kg), which often results in liver toxicity. Furthermore, because large doses are required when using conventional rAAVs, manufacturing sufficient amounts of a therapeutic rAAV needed to dose adult patients is extremely challenging. Additionally, due to differences in gene expression and physiology, mouse and primate models respond differently to viral capsids. Transduction efficiency of different virus particles varies between different species, and as a result, preclinical studies in mice often do not accurately reflect results in primates, including humans. As such there exists a need for improved rAAVs for use in the treatment of various genetic diseases.

Citation or identification of any document in this application is not an admission that such a document is available as prior art to the present invention.

### SUMMARY

Described in certain example embodiments herein are compositions comprising: a targeting moiety effective to target a muscle cell or both a muscle cell and a central nervous system (CNS) cell, wherein the targeting moiety comprises one or more n-mer inserts each comprising one or more P-motifs, wherein at least one P-motif comprises or consists of the amino acid sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7; or one or more RGD motifs, wherein at least one of the RGD motifs comprises or consists of XₘRGDXₙ, wherein m is 0-4 amino acids, wherein n is 0-15 amino acids, and wherein Xₘ, and Xₙ are each independently selected from any amino acid; or both, and optionally a cargo, wherein the cargo is coupled to or is otherwise associated with the targeting moiety.

In certain example embodiments, the one or more of the one or more P-motifs and one or more of the RGD motifs are each independently selected from any one set forth in one or more of SEQ ID NOs: 4-1698 (Tables 4-11).

In certain example embodiments, the one or more RGD motifs and/or the one or more P motifs are selected from any one or more set forth in: SEQ ID NOs: 4-250 (Table 4); SEQ ID NOs: 497-647 (Table 6); SEQ ID NOs: 799-1074 (Table 8); SEQ ID NOs: 1301-1497 (Table 10); or any combination thereof.

In certain example embodiments, the one or more RGD motifs and/or the one or more P motifs are selected from any one or more set forth in: SEQ ID NOs: 251-496 (Table 5); SEQ ID NOs: 648-798 (Table 7); SEQ ID NOs: 1498-1698 (Table 9); SEQ ID NOs: 1075-1300 (Table 11); or any combination thereof.

In certain example embodiments, the one or more RGD motifs and/or the one or more P motifs are selected from any one or more set forth in: SEQ ID NOs: 4-250 (Table 4) and/or SEQ ID NOs: 251-496 (Table 5); SEQ ID NOs: 497-647 (Table 6) and/or SEQ ID NOs: 648-798 (Table 7); SEQ ID NOs: 799-1074 (Table 8) and/or SEQ ID NOs: 1498-1698 (Table 9); or SEQ ID NOs: 1301-1497 (Table 10) and/or SEQ ID NOs: 1075-1300 (Table 11).

In certain example embodiments, the targeting moiety is effective target a skeletal muscle cell; a cardiac muscle cell; a skeletal muscle cell and a CNS cell; or a cardiac muscle cell and a CNS cell.

In certain example embodiments, the one or more n-mer inserts are each 3-25 or 3-15 amino acids in length.

In certain example embodiments: X₁ is S, T, or A; X₂ is L, V, F, or I; or both.

In certain example embodiments, the one or more RGD motifs and/or one or more P-motif is immediately preceded by AQ or DG in the targeting moiety.

In certain example embodiments, the targeting moiety comprises a polypeptide, a polynucleotide, a lipid, a polymer, a sugar, or a combination thereof.

In certain example embodiments, the targeting moiety comprises a viral protein.

In certain example embodiments, the viral protein is a capsid protein.

In certain example embodiments, one or more of the n-mer motifs are incorporated into the viral protein such that at least one of the one or more RGD motifs, at least one of the one or more P motifs, or both is/are located between two amino acids of the viral protein such that at least one of the one or more RGD motifs and/or one or more P-motifs is external to a viral capsid.

In certain example embodiments, the viral protein is an adeno associated virus (AAV) protein.

In certain example embodiments, the AAV protein is an AAV capsid protein.

In certain example embodiments, one or more of the one or more n-mer inserts are incorporated into the AAV protein such that at least one or more of the one more RGD motifs and/or one or more of the at least one or more P motifs are each inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

In certain example embodiments, at least one of the one or more n-mer inserts is incorporated into the AAV protein such that at least one of the one more RGD motifs and/or at least one of the one or more P motifs is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

In certain example embodiments, the AAV capsid protein is an engineered AAV capsid protein having reduced or eliminated uptake in a non-CNS and/or non-muscle cell as compared to a corresponding wild-type AAV capsid polypeptide.

In certain example embodiments, the non-CNS and/or non-muscle cell is a liver cell.

In certain example embodiments, the wild-type capsid polypeptide is an AAV1, AAV2, AAV3, AAV4, AAV5, AAV6, AAV7, AAV8, AAV9, AAV rh.74, or AAV rh.10 capsid polypeptide.

In certain example embodiments, the engineered AAV capsid protein comprises one or more mutations that result in reduced or eliminated uptake in a non-CNS and/or non-muscle cell.

In certain example embodiments, the one or more mutations are in position 267, in position 269, in position 504, in position 505, in position 590, or any combination thereof in the AAV9 capsid protein (SEQ ID NO: 1) or in one or more positions corresponding thereto in a non-AAV9 capsid polypeptide.

In certain example embodiments, the non-AAV9 capsid protein is an AAV1, AAV2, AAV3, AAV4, AAV5, AAV6, AAV7, AAV8, AAV rh.74, or AAV rh.10 capsid polypeptide.

In certain example embodiments the mutation in position 267 in the AAV9 capsid protein (SEQ ID NO: 1) or position corresponding thereto in a non-AAV9 capsid polypeptide is a G or X mutation to A, wherein X is any amino acid.

In certain example embodiments, the mutation in position 269 in the AAV9 capsid protein (SEQ ID NO: 1) or position corresponding thereto in a non-AAV9 capsid polypeptide is an S or X to T mutation, wherein X is any amino acid.

In certain example embodiments, the mutation in position 504 in the AAV9 capsid protein (SEQ ID NO: 1) or position corresponding thereto in a non-AAV9 capsid polypeptide is a G or X to A mutation, wherein X is any amino acid.

In certain example embodiments, the mutation in position 505 in the AAV9 capsid protein (SEQ ID NO: 1) or position corresponding thereto in a non-AAV9 capsid polypeptide is a P or X to A mutation, wherein X is any amino acid.

In certain example embodiments, the mutation in position 590 in the AAV9 capsid protein (SEQ ID NO: 1) or position corresponding thereto in a non-AAV9 capsid polypeptide is a Q or X to A mutation, wherein X is any amino acid.

In certain example embodiments, the engineered AAV capsid protein is an engineered AAV9 capsid polypeptide comprising a mutation at position 267, position 269 or both of a wild-type AAV9 capsid protein (SEQ ID NO: 1), wherein the mutation at position 267 is a G to A mutation and wherein the mutation at position 269 is an S to T mutation.

In certain example embodiments, the engineered AAV capsid protein is an engineered AAV9 capsid polypeptide comprising a mutation at position 590 of a wild-type AAV9 capsid protein (SEQ ID NO: 1), wherein the mutation at position 509 is a Q to A mutation.

In certain example embodiments, the engineered AAV capsid protein is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

In certain example embodiments, the composition is an engineered viral particle.

In certain example embodiments, the engineered viral particle is an engineered AAV viral particle.

In certain example embodiments, the AAV viral particle is an engineered AAV1, AAV2, AAV3, AAV4, AAV5, AAV6, AAV7, AAV8, AAV9, AAV rh.74, or AAV rh.10 viral particle.

In certain example embodiments, the optional cargo is capable of treating or preventing a CNS, a muscle disease or disorder, or both.

In certain example embodiments, the muscle disease and/or CNS disease or disorder is an auto immune disease; a cancer; a muscular dystrophy; a neuro-muscular disease; a sugar or glycogen storage disease; an expanded repeat disease; a dominant negative disease; a cardiomyopathy; a viral disease; a progeroid disease; or any combination thereof.

In certain example embodiments, the optional cargo is a morpholino, a peptide-linked morpholino, an antisense oligonucleotide, a PMO, a therapeutic transgene, a polynucleotide encoding a therapeutic polypeptide or peptide, a PPMO, one or more peptides, one or more polynucleotides encoding a CRISPR-Cas protein, a guide RNA, or both, a ribonucleoprotein, wherein the ribonucleoprotein comprises a CRISPR-Cas system molecule, a therapeutic transgene RNA, or other gene modifying or therapeutic RNA and/or protein, or any combination thereof.

In certain example embodiments, the optional cargo is capable of inducing exon skipping in a gene, optionally a dystrophin gene.

In certain example embodiments, the cargo is a mini- or micro-dystrophin gene.

In certain example embodiments, the mini- or micro-dystrophin gene comprises spectrin-like repeats 1, 2, 3, and 24, and optionally an nNOS domain.

In certain example embodiments, the expanded repeat disease is Huntington's disease, a Myotonic Dystrophy, or Facioscapulohumeral muscular dystrophy (FSHD).

In certain example embodiments, the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD.

In certain example embodiments, the myotonic dystrophy is Type 1 or Type 2.

In certain example embodiments, the cardiomyopathy is dilated cardiomyopathy, hypertrophic cardiomyopathy, DMD-associated cardiomyopathy, or Dannon disease.

In certain example embodiments, the sugar or glycogen storage disease is a MPS type III disease or Pompe disease. In certain example embodiments, the MPS type III disease, is MPS Type IIIA, IIIB, IIIC, or IIID.

In certain example embodiments, the neuro-muscular disease is Charcot-Marie-Tooth disease or Friedreich's Ataxia.

Described in certain example embodiments herein are vector systems comprising a vector comprising: one or more polynucleotides, wherein at least one of the one or more polynucleotides encodes all or part of a targeting moiety effective to target a muscle cell or both a muscle cell and a central nervous system (CNS) cell, wherein the targeting moiety comprises one or more n-mer inserts comprising one or more P-motifs, wherein at least one P-motif comprises or consists of the amino acid sequence XₘPX₁QGTX₂RXₙ, wherein X₁, X₂, Xₘ, and Xₙ are each independently selected from any amino acid, wherein m is 0, 1, 2, or 3, and wherein n is 0, 1, 2, 3, 4, 5, 6, or 7; or one or more RGD motifs, wherein at least one of the RGD motifs comprises or consists of XₘRGDXₙ, wherein m is 0-4 amino acids, wherein n is 0-15 amino acids, and wherein Xₘ, and Xₙ are each independently selected from any amino acid; or both, wherein at least one of the one or more polynucleotides encodes the at least one RGD motif, at least one P-motif, or both; and optionally, a regulatory element operatively coupled to one or more of the one or more polynucleotides.

In certain example embodiments, the one or more of the one or more P-motifs and one or more of the RGD motifs are each independently selected from any one set forth in any one or more of SEQ ID NOs: 4-1698 (Tables 4-11).

In certain example embodiments, the one or more RGD motifs and/or the one or more P motifs are selected from any one or more set forth in: SEQ ID NOs: 4-250 (Table 4); SEQ ID NOs: 497-647 (Table 6); SEQ ID NOs: 799-1074 (Table 8); SEQ ID NOs: 1301-1497 (Table 10); or any combination thereof

In certain example embodiments, the one or more RGD motifs and/or the one or more P motifs are selected from any one or more set forth in: SEQ ID NOs: 251-496 (Table 5); SEQ ID NOs: 648-798 (Table 7); SEQ ID NOs: 1498-1698 (Table 9); SEQ ID NOs: 1075-1300 (Table 11); or any combination thereof

In certain example embodiments, the one or more RGD motifs and/or the one or more P motifs are selected from any one or more set forth in: SEQ ID NOs: 4-250 (Table 4) and/or SEQ ID NOs: 251-496 (Table 5); SEQ ID NOs: 497-647 (Table 6) and/or SEQ ID NOs: 648-798 (Table 7); SEQ ID NOs: 799-1074 (Table 8) and/or SEQ ID NOs: 1498-1698 (Table 9); or SEQ ID NOs: 1301-1497 (Table 10) and/or SEQ ID NOs: 1075-1300 (Table 11).

In certain example embodiments, the targeting moiety is effective at targeting a skeletal muscle cell; a cardiac muscle cell; a skeletal muscle cell and a CNS cell; or a cardiac muscle cell and a CNS cell.

In certain example embodiments, the one or more n-mer motifs are each 3-25 or 3-15 amino acids in length.

In certain example embodiments, X₁ is S, T, or A; X₂ is L, V, F, or I, or both.

In certain example embodiments, the n-mer insert is immediately preceded by AQ or DG in the targeting moiety.

In certain example embodiments, the vector system further comprises a cargo.

In certain example embodiments, the cargo is a cargo polynucleotide and is optionally operatively coupled to one or more of the one or more polynucleotides encoding all or part of the targeting moiety.

In certain example embodiments, the vector system is capable of producing virus particles, virus particles that contain the cargo, or both.

In certain example embodiments, the vector system is capable of producing a polypeptide comprising one or more of the targeting moieties.

In certain example embodiments, the polypeptide is a viral polypeptide.

In certain example embodiments, the viral polypeptide is a capsid polypeptide.

In certain example embodiments, the capsid polypeptide is an adeno associated virus (AAV) capsid polypeptide.

In certain example embodiments, the virus particles are AAV virus particles.

In certain example embodiments, the AAV virus particles or AAV capsid polypeptide are engineered AAV1, AAV2, AAV3, AAV4, AAV5, AAV6, AAV7, AAV8, AAV9, AAV rh.74, or AAV rh.10 viral particles or polypeptides.

In certain example embodiments, one or more of the one or more n-mer inserts are incorporated in the targeting moiety such that at least one of the one or more RGD motifs, at least one of the one or more P motifs, or both is/are located between two amino acids of the viral protein such that at least one of the one or more RGD motifs and/or one or more P-motifs is external to a viral capsid of the virus particles.

In certain example embodiments, one or more of the one or more n-mer inserts are incorporated into the AAV protein such that at least one or more of the one more RGD motifs and/or at least one or more of the one or more P motifs are each inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

In certain example embodiments, at least one of the one or more n-mer inserts is incorporated into the AAV protein such that at least one of the one more RGD motifs and/or at least one of the one or more P motifs is inserted between amino acids 588 and 589 in the AAV9 capsid polynucleotide or in an analogous position in an AAV1, AAV2, AAV3, AAV4, AAV5, AAV6, AAV7, AAV8, AAV rh.74, AAV rh.10 capsid polypeptide.

In certain example embodiments, the AAV capsid protein is an engineered AAV capsid protein having reduced or eliminated uptake in a non-CNS cell or a non-muscle cell as compared to a corresponding wild-type AAV capsid polypeptide.

In certain example embodiments, the non-CNS or non-muscle cell is a liver cell.

In certain example embodiments, the wild-type capsid polypeptide is an AAV1, AAV2, AAV3, AAV4, AAV5, AAV6, AAV7, AAV8, AAV9, AAV rh.74, or AAV rh.10 capsid polypeptide.

In certain example embodiments, wherein the engineered AAV capsid protein comprises one or more mutations that result in reduced or eliminated uptake in a non-CNS cell.

In certain example embodiments, the one or more mutations are in position 267, in position 269, in position 504, in position 505, in position 590, or any combination thereof in the AAV9 capsid protein (SEQ ID NO: 1) or in one or more positions corresponding thereto in a non-AAV9 capsid polypeptide.

In certain example embodiments, the non-AAV9 capsid protein is an AAV1, AAV2, AAV3, AAV4, AAV5, AAV6, AAV7, AAV8, AAV rh.74, or AAV rh.10 capsid polypeptide.

In certain example embodiments, the mutation in position 267 in the AAV9 capsid protein (SEQ ID NO: 1) or position corresponding thereto in a non-AAV9 capsid polypeptide is a G or X mutation to A, wherein X is any amino acid.

In certain example embodiments, the mutation in position 269 in the AAV9 capsid protein (SEQ ID NO: 1) or position corresponding thereto in a non-AAV9 capsid polypeptide is an S or X to T mutation, wherein X is any amino acid.

In certain example embodiments, the mutation in position 504 in the AAV9 capsid protein (SEQ ID NO: 1) or position corresponding thereto in a non-AAV9 capsid polypeptide is a G or X to A mutation, wherein X is any amino acid.

In certain example embodiments, the mutation in position 505 in the AAV9 capsid protein (SEQ ID NO: 1) or position corresponding thereto in a non-AAV9 capsid polypeptide is a P or X to A mutation, wherein X is any amino acid.

In certain example embodiments, the mutation in position 590 in the AAV9 capsid protein (SEQ ID NO: 1) or position corresponding thereto in a non-AAV9 capsid polypeptide is a Q or X to A mutation, wherein X is any amino acid.

In certain example embodiments, the engineered AAV capsid protein is an engineered AAV9 capsid polypeptide comprising a mutation at position 267, position 269 or both of a wild-type AAV9 capsid protein (SEQ ID NO: 1), wherein the mutation at position 267 is a G to A mutation and wherein the mutation at position 269 is an S to T mutation.

In certain example embodiments, the engineered AAV capsid protein is an engineered AAV9 capsid polypeptide comprising a mutation at position 590 of a wild-type AAV9 capsid protein (SEQ ID NO: 1), wherein the mutation at position 509 is a Q to A mutation.

In certain example embodiments, the engineered AAV capsid protein is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

In certain example embodiments, the vector comprising the one or more polynucleotides does not comprise splice regulatory elements.

In certain example embodiments, the vector further comprises a polynucleotide that encodes a viral rep protein.

In certain example embodiments, the viral rep protein is an AAV rep protein.

In certain example embodiments, the polynucleotide that encodes the viral rep protein is on the same vector or a different vector as the one or more polynucleotides.

In certain example embodiments, the polynucleotide that encodes the viral rep protein is operatively coupled to a regulatory element.

In certain example embodiments, the vector system is capable of producing a composition or portion thereof as described in any of the above paragraphs and/or elsewhere herein.

Described in certain example embodiments are polypeptide encoded, produced, or both by a vector system as described in any one or more of the paragraphs above or elsewhere herein.

In certain example embodiments, the polypeptide is a viral polypeptide.

In certain example embodiments, the viral polypeptide is an AAV polypeptide.

In certain example embodiments, the polypeptide is coupled to or otherwise associated with a cargo.

Described in certain example embodiments are particles produced by a vector system as in any one of claims 48-88, optionally including a polypeptide as in any one of the above paragraphs or elsewhere herein.

In certain example embodiments, the particle is a viral particle.

In certain example embodiments, the viral particle is an adeno-associated virus (AAV) particle, lentiviral particle, or a retroviral particle.

In certain example embodiments, the particle comprises a cargo.

In certain example embodiments, the viral particle has a muscle tropism, or a muscle and central nervous system (CNS) tropism.

In certain example embodiments, the cargo is capable or preventing a CNS disease or, a muscle disease or disorder, or both a CNS and muscle disease or disorder.

In certain example embodiments, the CNS or muscle disease or disorder is (a) an auto immune disease; (b) a cancer; (c) a muscular dystrophy; (d) a neuro-muscular disease; (e) a sugar or glycogen storage disease; (f) an expanded repeat disease; (g) a dominant negative disease; (h) a cardiomyopathy; (i) a viral disease; (j) a progeroid disease; or (k) any combination thereof.

In certain example embodiments, the cargo is capable of inducing exon skipping in a gene.

In certain example embodiments, the cargo is capable of inducing exon skipping in a dystrophin gene.

In certain example embodiments, the cargo is a mini- or micro-dystrophin gene.

In certain example embodiments, the mini- or micro-dystrophin gene comprises spectrin-like repeats 1, 2, 3, and 24, and optionally an nNOS domain.

In certain example embodiments, the expanded repeat disease is Huntington's disease, a Myotonic Dystrophy, or Facioscapulohumeral muscular dystrophy (FSHD).

In certain example embodiments, the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD.

In certain example embodiments, the myotonic dystrophy is Type 1 or Type 2.

In certain example embodiments, the cardiomyopathy is dilated cardiomyopathy, hypertrophic cardiomyopathy, DMD-associated cardiomyopathy, or Dannon disease.

In certain example embodiments, the sugar or glycogen storage disease is a MPS type III disease or Pompe disease.

In certain example embodiments, wherein the MPS type III disease, is MPS Type IIIA, IIIB, IIIC, or IIID.

In certain example embodiments, the neuro-muscular disease is Charcot-Marie-Tooth disease or Friedreich's Ataxia.

In certain example embodiments, the polypeptide, the particle, or both have increased muscle cell potency, muscle cell specificity, reduced immunogenicity, or any combination thereof

Described in certain example embodiments are cells comprising: a composition as in any one of the above paragraphs and elsewhere herein; a vector system as in any one of the above paragraphs and elsewhere herein; a polypeptide as in any one of the above paragraphs and elsewhere herein; a particle as in any one of the above paragraphs and elsewhere herein; or a combination thereof.

In certain example embodiments, the cell is prokaryotic. In certain example embodiments, the cell is eukaryotic.

Described in certain example embodiments herein are pharmaceutical formulations comprising: a composition as in any one of the above paragraphs and elsewhere herein; a vector system as in any one of the above paragraphs and elsewhere herein; a polypeptide as in any one of the above paragraphs and elsewhere herein; a particle as in any one of the above paragraphs and elsewhere herein; a cell as in any one of the above paragraphs and elsewhere herein; or a combination thereof; and a pharmaceutically acceptable carrier.

Described in certain example embodiments herein are methods of treating a muscle disease, disorder, or a symptom thereof, or both a muscle and a central nervous system disease, disorder, or a symptom thereof comprising: administering, to the subject in need thereof, a composition as in any one of the above paragraphs and elsewhere herein a vector system as in any one of the above paragraphs and elsewhere herein; a polypeptide as in any one of the above paragraphs and elsewhere herein a particle as in any one of the above paragraphs and elsewhere herein a cell as in any one of the above paragraphs and elsewhere herein; a pharmaceutical formulation as in any one of the above paragraphs and elsewhere herein; or a combination thereof.

In certain example embodiments, the central nervous system disease or disorder comprises a secondary muscle disease, disorder, or symptom thereof.

In certain example embodiments, the central nervous system disease or disorder is Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, or a combination thereof.

In certain example embodiments, the CNS or muscle disease or disorder is (a) an auto immune disease; (b) a cancer; (c) a muscular dystrophy; (d) a neuro-muscular disease; (e) a sugar or glycogen storage disease; (f) an expanded repeat disease; (g) a dominant negative disease; (h) a cardiomyopathy; (i) a viral disease; (j) a progeroid disease; or (k) any combination thereof.

In certain example embodiments, the expanded repeat disease is Huntington's disease, a Myotonic Dystrophy, or Facioscapulohumeral muscular dystrophy (FSHD).

In certain example embodiments, the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD.

In certain example embodiments, the myotonic dystrophy is Type 1 or Type 2.

The method of any one of claims 116-122, wherein the cardiomyopathy is dilated cardiomyopathy, hypertrophic cardiomyopathy, DMD-associated cardiomyopathy, or Dannon disease.

In certain example embodiments, the sugar or glycogen storage disease is a MPS type III disease or Pompe disease.

In certain example embodiments, the MPS type III disease, is MPS Type IIIA, IIIB, IIIC, or IIID.

In certain example embodiments, the neuro-muscular disease is Charcot-Marie-Tooth disease or Friedreich's Ataxia.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows the adeno-associated virus (AAV) transduction mechanism, which results in production of mRNA from the transgene.

FIG. 2 shows a graph that can demonstrate that mRNA-based selection of AAV variants can be more stringent than DNA-based selection. The virus library was expressed under the control of a CMV promoter.

FIGS. 3A-3B show graphs that can demonstrate a correlation between the virus library and vector genome DNA (FIG. 3A) and mRNA (FIG. 3B) in the liver.

FIGS. 4A-4F show graphs that can demonstrate capsid variants present at the DNA level and expressed at the mRNA level identified in different tissues. For this experiment, the virus library was expressed under the control of a CMV promoter.

FIGS. 5A-5C show graphs that can demonstrate capsid mRNA expression in different tissues under the control of cell-type specific promoters (as noted on x-axis). CMV was included as an exemplary constitutive promoter. CK8 is a muscle-specific promoter. MHCK7 is a muscle-specific promoter. hSyn is a neuron specific promoter. Expression levels from the cell type-specific promoters have been normalized based on expression levels from the constitutive CMV promoter in each tissue.

FIGS. 6A-6B show (FIG. 6A) a schematic demonstrating embodiments of a method of producing and selecting capsid variants for tissue-specific gene delivery across species and (FIG. 6B) a schematic demonstrating benchmarking of the top selected capsids.

FIG. 7 shows a schematic demonstrating embodiments of generating an AAV capsid variant library, particularly insertion of a random n-mer (n=3-15 amino acids) into a wild-type AAV, e.g., AAV9.

FIG. 8 shows a schematic demonstrating embodiments of generating an AAV capsid variant library, particularly variant AAV particle production. Each capsid variant encapsulates its own coding sequence as the vector genome.

FIG. 9 shows schematic vector maps of representative AAV capsid plasmid library vectors (see e.g., FIG. 8) that can be used in an AAV vector system to generate an AAV capsid variant library.

FIG. 10 shows a graph that can demonstrate the viral titer (calculated as AAV9 vector genome/15 cm dish) produced by constructs containing different constitutive and cell-type specific mammalian promoters.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide targeting moieties with muscle specificity (e.g., cardiac and/or skeletal muscle (collectively referred to as “muscle-specific” targeting moieties). Embodiments disclosed herein also provide targeting moieties with dual specificity for central nervous system (CNS) and skeletal muscle or CNS and cardiac muscle (collectively referred to as “CNS-muscle specific targeting moieties”). The targeting moieties can be coupled to or otherwise associated with a cargo and/or delivery vehicle or system. Embodiments disclosed herein provide polypeptides and particles that can incorporate one or more of the CNS-muscle- or muscle-specific targeting moieties. The polypeptides and/or particles can be coupled to, attached to, encapsulate, or otherwise incorporate a cargo, thereby associating the cargo with the targeting moiety(ies). Embodiments disclosed herein provide CNS-muscle- or muscle-specific targeting moieties that can contain one or more of an n-mer insert that can contain or be an RGD-motif and/or P-motif as further described herein.

In some embodiments, the n-mer insert(s) is or contains one or more RGD motifs and/or P-motifs. In some embodiments, one or more of the RGD motifs and/or P-motifs is as set forth in any one or more of Tables 4-11 (SEQ ID NOS: 4-1300), any one or more of Tables 4, 6, 8, and/or 10; any one or more of Tables 5, 7, 9, and/or 11; and/or any one or more of Tables 4 and/or 5; any one or more of Tables 6 and/or 7; any one or more of Tables 8 and/or 9; any one or more of Tables 10 and/or 11, or any combination thereof. In some embodiments, at least one P-motif comprises or consists of the amino acid sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7. In some embodiments, at least one of the RGD motifs comprises or consists of XₘRGDXₙ, wherein m is 0-4 amino acids, wherein n is 0-15 amino acids, and wherein Xₘ, and Xₙ are each independently selected from any amino acid. In some embodiments, the muscle specific targeting moieties comprise one or more RGD motifs. In some embodiments, the targeting moieties that have CNS and skeletal and/or cardiac muscle specificity comprise a P-motif, an RGD motif, or both.

Embodiments disclosed herein provide engineered viral (e.g., adeno-associated virus (AAV)) capsids that can be engineered to confer cell-specific and/or species-specific tropism, such as CNS-muscle or muscle (e.g., a cardiac muscle and/or skeletal muscle) tropism, to an engineered viral (e.g., AAV) particle. Embodiments disclosed herein provide engineered viral polypeptides (e.g., capsid polypeptides) that can include one or more targeting moieties described herein. In some embodiments, the engineered viral polypeptides can be incorporated into viral particles.

Embodiments disclosed herein also provide methods of generating recombinant AAVs (rAAVs) having engineered capsids that can involve systematically directing the generation of diverse libraries of variants of modified surface structures, such as variant capsid proteins. Embodiments of the method of generating rAAVs having engineered capsids can also include stringent selection of capsid variants capable of targeting a specific cell, tissue, and/or organ type. Embodiments of the method of generating rAAVs having engineered capsids can include stringent selection of capsid variants capable of efficient and/or homogenous transduction in at least two or more species. In one example embodiment, the n-mer insert may result in increased transduction of muscle cells (e.g., cardiac and/or skeletal muscle cells) or both CNS and muscle cells (e.g., cardiac and/or skeletal muscle cells).

Embodiments disclosed herein provide vectors and systems thereof capable of producing an engineered AAV described herein.

Embodiments disclosed herein provide cells that can be capable of producing the engineered AAV particles described herein. In some embodiments, the cells include one or more vectors or system thereof described herein.

Embodiments disclosed herein provide engineered AAVs that can include an engineered capsid described herein. In some embodiments, the engineered AAV can include a cargo polynucleotide to be delivered to a cell. In some embodiments, the cargo polynucleotide is a gene modification polynucleotide.

Embodiments disclosed herein provide formulations that can contain an engineered AAV vector or system thereof, an engineered AAV capsid, engineered AAV particles including an engineered AAV capsid described herein, and/or an engineered cell described herein that contains an engineered AAV capsid, and/or an engineered AAV vector or system thereof. In some embodiments, the formulation can also include a pharmaceutically acceptable carrier. The formulations described herein can be delivered to a subject in need thereof or a cell.

Embodiments disclosed herein also provide kits that contain one or more of the one or more of the polypeptides, polynucleotides, vectors, engineered AAV capsids, engineered AAV particles, cells, or other components described herein and combinations thereof and pharmaceutical formulations described herein. In embodiments, one or more of the polypeptides, polynucleotides, vectors, engineered AAV capsids, engineered AAV particles cells, and combinations thereof described herein can be presented as a combination kit.

Embodiments disclosed herein provide methods of using the engineered AAVs having a cell-specific tropism described herein to deliver, for example, a therapeutic polynucleotide to a cell. In this way, the engineered AAVs described herein can be used to treat and/or prevent a disease in a subject in need thereof. Embodiments disclosed herein also provide methods of delivering the engineered AAV capsids, engineered AAV virus particles, engineered AAV vectors or systems thereof and/or formulations thereof to a cell. Also provided herein are methods of treating a subject in need thereof by delivering an engineered AAV particle, engineered AAV capsid, engineered AAV capsid vector or system thereof, an engineered cell, and/or formulation thereof to the subject.

Additional features and advantages of the embodiments engineered AAVs and methods of making and using the engineered AAVs are further described herein.

### Targeting Moieties and Compositions

Generally, described herein are compositions containing one or more CNS-muscle specific or muscle specific (e.g., cardiac muscle specific and/or skeletal muscle specific) targeting moieties that can effectively target muscle cells or CNS and muscle cells. In some embodiments, one or more CNS-muscle specific or muscle specific targeting moieties can be incorporated into a delivery vehicle, agent, or system thereof so as to provide CNS-muscle specific or muscle specific targeting capability to the delivery vehicle, agent, or system thereof. Exemplary delivery vehicles include, without limitation, viral particles, (e.g., AAV viral particles), micelles, liposomes, exosomes, and the like. Exemplary delivery vehicles in which the CNS-muscle specific or muscle specific targeting-moieties can be incorporated are described in greater detail elsewhere herein. In some embodiments, the CNS-muscle specific or muscle specific-targeting moieties can be indirectly or directly coupled to a cargo and thus provide CNS-muscle specific or muscle specific to the coupled cargo. In some embodiments, the composition can be specific for CNS and muscle cells or a muscle cell (e.g., as conferred by the CNS-muscle specific or muscle specific targeting moieties described herein) and have reduced specificity for a non-CNS or a non-muscle cell (including, but not limited to, a liver cell). In some embodiments, the CNS-muscle specific or muscle specific targeting moiety can specifically interact with or otherwise associate with one or more AAV receptors on CNS and/or muscle cells, thus providing CNS and muscle or muscle specificity (or tropism). Methods of generating and identifying CNS-muscle specific or muscle specific targeting moieties are described in greater detail elsewhere herein.

### CNS and Muscle Specific or Muscle Specific Targeting Moieties

Described herein are targeting moieties capable of specifically targeting, a muscle (e.g., a cardiac muscle cell and/or skeletal muscle) cell, or both muscle cells and CNS cells. As used in this context herein, “targeting” refers to the ability to, in a target specific manner, recognize, bind, associate with, transduce or infect, or otherwise interact with a target molecule or moiety such that recognition, binding, association, affinity, avidity, transduction or infection, and/or other interaction with the target molecule or moiety by the targeting moiety is greater, more efficient, or otherwise more selective for the target molecule or moiety as compared with its recognition, binding, association, affinity, avidity, transduction or infection, and/or other interaction with a non-target molecule or moiety. For example, a CNS-specific targeting moiety has increased and/or more efficient or selective recognition, binding, association, affinity, avidity, transduction or infection, and/or other interaction of or with CNS cells as compared to non-CNS cells. Likewise, a muscle-specific targeting moiety has increased and/or more efficient or selective recognition, binding, association, affinity, avidity, transduction or infection, and/or other interaction of or with muscle cells as compared to non-muscle cells. A targeting moiety that is both CNS and muscle cell specific has increased and/or more efficient or selective recognition, binding, association, affinity, avidity, transduction or infection, and/or other interaction of or with CNS cells as compared to non-CNS and non-muscle cells.

In some embodiments, the targeting moiety can be or include one or more n-mer inserst described herein. The n-mer insert can be (consists of) or contain (comprises) an RGD motif, a P-motif, or both. Generally, n-mer inserts are short (e.g., about 3 to about 15, 20, or 25) amino acid sequences where each amino acid of the n-mer insert can be selected from any amino acid. In some embodiments, the n-mer insert is 3, 4, 5, 6, 7, 8, 9, 10, 11, 12, 13, 14, 15, 16, 17, 18, 19, 20, 21, 22, 23, 24, or 25 amino acids in length

In some embodiments, the n-mer insert is or contains a P-motif The term “P-motif” as used herein refers to an amino acid having the sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7. In some embodiments, the P-motif has the amino acid sequence PX₁QGTX₂RXₙ, where X₁, X₂, Xₙ, are each selected from any amino acid and where n is 0, 1, 2, 3, 4, 5, 6, or 7. Exemplary P-motifs are described in greater detail elsewhere herein.

In some embodiments, the n-mer insert is (consists of) or include (comprises) an “RGD” motif. An “RGD” motif generally refers to the presence of the amino acids RGD within the n-mer insert. In some embodiments, the RGD is the first three amino acids of the n-mer insert. Thus, in some embodiments the n-mer can have a sequence of RGD or RGDXₙ, where n can be 3-15 amino acids, where X can be any amino acid, and where each amino acid present can each be independently selected from the others from the group of: any amino acid. In some embodiments, the n-mer insert can be RGD (3-mer), RGDX₁ (4-mer), RGDX₁X₂ (5-mer), RGDX₁X₂X₃ (6-mer), RGDX₁X₂X₃X₄ (7 mer), RGDX₁X₂X₃X₄X₅ (8 mer), or RGDX₁X₂X₃X₄X₅X₆ (9-mer), RGD₁X₂X₃X₄X₅X₆X₇ (10-mer), RGD₁X₂X₃X₄X₅X₆X₇X₈ (11-mer), RGDX₁X₂X₃X₄X₅X₆X₇X₈X₉ (12-mer), RGDX₁X₂X₃X₄X₅X₆X₇X₈X₉X₁₀ (13-mer), RGDX₁X₂X₃X₄X₅X₆X₇X₈X₉X₁₀X₁₁ (14-mer), or RGDX₁X₂X₃X₄X₅X₆X₇X₈X₉X₁₀X₁₁X₁₂ (15-mer), where X₁, X₂, X₃, X₄, X₅, X₆, X₇, X₈, X₉, X₁₀, X₁₁, X₁₂ can each be independently selected and can be any amino acid. In some embodiments, X₁ can be L, T, A, M, V, Q, or M. In some embodiments, X₂ can be T, M, S, N, L, A, or I. In some embodiments, X₃ can be T, E, N, O, S, Q, Y, A, or D. In some embodiments, X₄ can be P, Y, K, L, H, T, or S.

In certain example embodiments, the RGD motif has a formula of XₘRGDXₙ, wherein m is 0-4 amino acids, wherein n is 0-15 amino acids, wherein X is any amino acid, and wherein each X amino acid present is independently selected from the others from the group of: any amino acid. In certain example embodiments, the RGD motif has the formula RGDXₙ, wherein n is 4 or 5, wherein X is any amino acid, and wherein each X amino acid present is independently selected from the others from the group consisting of: any amino acid or any specific combinations described elsewhere herein.

In some embodiments, one or more of the n-mer inserts, RGD motifs, and/or P-motifs is or includes a motif as set forth in in any one or more of Tables 4-11 (SEQ ID NOS: 4-1300), any one or more of Tables 4, 6, 8, and/or 10; any one or more of Tables 5, 7, 9, and/or 11; and/or any one or more of Tables 4 and/or 5; any one or more of Tables 6 and/or 7; any one or more of Tables 8 and/or 9; any one or more of Tables 10 and/or 11, or any combination thereof. In some embodiments, at least one P-motif comprises or consists of the amino acid sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7. In some embodiments, at least one P-motif has the amino acid sequence PX₁QGTX₂RXₙ (SEQ ID NO: 1699), where X₁, X₂, Xₙ, are each selected from any amino acid and where n is 0, 1, 2, 3, 4, 5, 6, or 7. In some embodiments, at least one of the RGD motifs comprises or consists of XₘRGDXₙ, wherein m is 0-4 amino acids, wherein n is 0-15 amino acids, and wherein Xₘ, and Xₙ are each independently selected from any amino acid. In some embodiments, the muscle specific targeting moieties comprise one or more RGD motifs. In some embodiments, the targeting moieties that have CNS and skeletal and/or cardiac muscle specificity comprise a P-motif, an RGD motif, or both.

In some embodiments, n-mer inserts that are or include an RGD insert and/or P-motif is included in a CNS-muscle or muscle specific targeting moiety and can facilitate muscle or muscle and CNS targeting by the targeting moiety. As will be appreciated in view of the present disclosure, such targeting moieties with CNS and muscle targeting capabilities can be advantageous for use in compositions and formulations for treating CNS diseases with muscle cell (such as cardiac and/or skeletal muscle) involvement or pathologies. In some exemplary embodiments and as further discussed herein the targeting moiety can be a viral capsid such as an AAV viral capsid.

In some embodiments, the n-mer insert is not or does not include and RGD insert.

In some embodiments, the n-mer insert is not or does not include a P-motif.

In some embodiments, the n-mer insert, the RGD motif, and/or P-motif is immediately preceded by an AQ a DG in the targeting moeity, which can be part of the n-mer insert or part of another polypeptide into which the n-mer insert is incorporated, RGD motif, and/or P-motif is inserted, such as a vector (e.g., an AAV vector), or viral protein (e.g., viral capsid polypeptide). In some embodiments, the AQ or DG is incorporated in the n-mer insert preceding the RGD and/or P motif and replaces one or two amino acids of the polypeptide into which the n-mer insert is incorporated. In some embodiments, where an n-mer insert is a P motif having the sequence amino acid sequence XₘPX₁QGTX₂RXₙ (SEQ ID NO: 1699), and Xₘ in the P motif is not 0 (i.e., m=1, 2 or 3) the amino acids of Xₘ can replace up to 1, 2, or 3, respectively amino acids of the polypeptide into which the n-mer insert is being incorporated. Likewise, in some embodiments where an n-mer insert is an RGD motif having the sequence XₘRGDXₙ and n is not 0 (i.e., n=1, 2, 3 or 4), the amino acids of X. can replace up to 1, 2, 3, or 4, respectively, amino acids of the polypeptide into which the n-mer insert is being incorporated. In some embodiments, the polypeptide into which such an n-mer insert is being incorporated is a viral polypeptide, such as a viral capsid polypeptide (e.g., an AAV capsid polypeptide). Incorporation of an n-mer insert in this manner can position an RGD motif and/or P motif as an “insertion” between any two desired contiguous amino acids of the recipient polypeptide.

In some embodiments, X₁ of the P-motif is S, T, or A. In some embodiments, X₂ of the P-motif L, V, F, or I. In some embodiments, Xₙ of the P-motif is 0. In some embodiments, Xₙ of the P-motif is 1. In some embodiments, Xₙ of the P-motif is 2. In some embodiments, Xₙ of the P-motif is 3. In some embodiments, Xₙ of the P-motif is 4. In some embodiments, Xₙ of the P-motif is 5. In some embodiments, Xₙ of the P-motif is 6. In some embodiments, Xₙ of the P-motif is 7. In some embodiments, Xₘ of the P-motif is 0. In some embodiments, Xₘ of the P motif is 3. In some embodiments, Xₘ of the P motif is 2. In some embodiments, Xₘ of the P motif is 1.

In some embodiments, an n-mer insert having both muscle and CNS specificity (“CNS muscle-specificity”) is in any one or more of Tables 8-11. In some embodiments, a muscle specific n-mer insert is in any one or more of Tables 4-7. In some embodiments, a cardiac muscle specific n-mer insert is in any one or more of Tables 6 and/or 7. In some embodiments, a skeletal muscle specific n-mer insert is in any one or more of Tables 4 and/or 5. In some embodiments, a skeletal muscle specific n-mer insert is in any one or more of Tables 4 and/or 5. In some embodiments, a cardiac muscle specific n-mer insert is in any one or more of Tables 7 and/or 8.

In some embodiments, the CNS-muscle specific or muscle specific n-mer insert, RGD-motif, and/or P-motif is species specific. In other words, in some embodiments, the CNS-muscle specific or muscle specific n-mer insert and/or P-motif can facilitate CNS and muscle targeting or muscle targeting, respectively, in one species better than another species. In some embodiments the CNS-muscle specific or muscle n-mer insert is specific for primates. In some embodiments, the CNS-muscle specific or muscle n-mer insert is specific for human and/or non-human primates.

In some embodiments, the CNS-muscle specific or muscle n-mer insert is capable of targeting one or more cell and/or tissue types over others within the organism, such as the CNS and/or muscle types (e.g., cardiac, skeletal, or smooth). In some embodiments, the cardiac muscle specific n-mer insert is capable of targeting cardiac muscle cells over non-muscle cells and over skeletal and/or smooth muscle cells. In some embodiments, the cardiac muscle specific n-mer insert is capable of targeting skeletal muscle cells over non-muscle cells and over cardiac and/or smooth muscle cells. In some embodiments, the CNS-muscle specific n-mer insert is capable of targeting muscle and CNS cells over non-muscle and non-CNS cells. In some embodiments, the CNS-cardiac muscle specific n-mer insert is capable of targeting cardiac muscle and CNS cells over non-cardiac muscle and non-CNS cells. In some embodiments, the CNS-skeletal muscle specific n-mer insert is capable of targeting muscle and CNS cells over non-skeletal muscle and non-CNS cells.

In some embodiments, the targeting moiety can include more than one n-mer insert s, such as a CNS-muscle specific or muscle specific (e.g., a cardiac muscle specific or skeletal muscle specific) n-mer insert described herein. In some embodiments, the targeting moiety can include 1, 2, 3, 4, 5, 6, 7, 8, 9, 10 or more n-mer inserts. In some embodiments, all the n-motifs included in the targeting moiety can be the same. In some embodiments where more than one n-mer insert is included, at least two of the n-mer inserts are different from each other. In some embodiments where more than one n-mer insert is included, all the n-mer inserts are different from each other.

In some embodiments, the targeting moiety, e.g., the CNS-muscle specific or muscle specific targeting moiety, can be coupled to or otherwise associated with a cargo. In some embodiments, one or more muscle-specific targeting moieties described herein is directly attached to the cargo. In some embodiments, one or more muscle-specific targeting moieties described herein is indirectly coupled to the cargo, such as via a linker molecule. In some embodiments, one or more one or more muscle-specific targeting moieties described herein is coupled to associated with a polypeptide or other particle that is coupled to, attached to, encapsulates, and/or contains a cargo.

Exemplary particles include, without limitation, viral particles (e.g., viral capsids, which is inclusive of bacteriophage capsids), polysomes, liposomes, nanoparticles, microparticles, exosomes, micelles, and the like. The term “nanoparticle” as used herein includes a nanoscale deposit of a homogenous or heterogeneous material. Nanoparticles may be regular or irregular in shape and may be formed from a plurality of co-deposited particles that form a composite nanoscale particle. Nanoparticles may be generally spherical in shape or have a composite shape formed from a plurality of co-deposited generally spherical particles. Exemplary shapes for the nanoparticles include, but are not limited to, spherical, rod, elliptical, cylindrical, disc, and the like. In some embodiments, the nanoparticles have a substantially spherical shape.

As used herein, the term “specific” when used in relation to described an interaction between two moieties, refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

In some embodiments, in addition to the n-mer insert(s) the targeting moiety can include a polypeptide, a polynucleotide, a lipid, a polymer, a sugar, or a combination thereof.

In some embodiments, the targeting moiety is incorporated into a viral protein, such as a capsid protein, including but not limited to lentiviral, adenoviral, AAV, bacteriophage, retroviral proteins. In some embodiments, n-mer insert is located between two amino acids of the viral protein such that the n-mer insert is external (i.e., is presented on the surface of) to a viral capsid.

In some embodiments, the composition containing one or more of the muscle-specific targeting moieties described herein has increased muscle cell potency, muscle cell specificity, increased muscle cell tropism and/or transduction efficiency, reduced immunogenicity, or any combination thereof.

In some embodiments, the composition containing one or more of the CNS-muscle-specific targeting moieties described herein has increased muscle and CNS cell potency, CNS and muscle cell specificity, increased CNS and muscle cell tropism and/or transduction efficiency, reduced immunogenicity, or any combination thereof.

Cargos can include any molecule that is capable of being coupled to or associated with the muscle-specific targeting moieties described herein. Cargos can include, without limitation, nucleotides, oligonucleotides, polynucleotides, amino acids, peptides, polypeptides, riboproteins, lipids, sugars, pharmaceutically active agents (e.g., drugs, imaging and other diagnostic agents, and the like), chemical compounds, and combinations thereof. In some embodiments, the cargo is DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, radiation sensitizers, chemotherapeutics, radioactive compounds, imaging agents, and combinations thereof.

The CNS-specific n-mer inserts and targeting moieties can be encoded in whole or in part by a polynucleotide. The encoding polynucleotides can be included in one or more vectors (or vector systems) that can be used to generate targeting moieties and compositions thereof that include the CNS-muscle specific and/or muscle specific n-mer insert(s) described herein. Exemplary encoding polynucleotides, vectors, vector systems, and recombinant engineering techniques are described in greater detail herein and/or are generally known in the art and can be adapted for use with the targeting moieties and compositions thereof described herein.

In some embodiments, the cargo is capable of treating or preventing a CNS and/or muscle disease or disorder. Exemplary CNS and muscle diseases and disorders are described elsewhere herein.

### Cargos

Representative cargo molecules that may be delivered using the compositions disclosed herein include, but are not limited to, nucleic acids, polynucleotides, proteins, polypeptides, polynucleotide/polypeptide complexes, small molecules, sugars, or a combination thereof. Cargos that can be delivered in accordance with the systems and methods described herein include, but are not necessarily limited to, biologically active agents, including, but not limited to, therapeutic agents, imaging agents, and monitoring agents. A cargo may be an exogenous material or an endogenous material. In some embodiments, the cargo can be a “gene of interest”.

### Polynucleotides

In some embodiments, the cargo is a cargo polynucleotide. As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically, or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA, including but not limited to, tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), or coding mRNA (messenger RNA).

In some embodiments, the cargo polynucleotide is DNA. In some embodiments, the cargo polynucleotide is RNA. In some embodiments, the cargo polynucleotide is a polynucleotide (a DNA or an RNA) that encodes an RNA and/or a polypeptide. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Genes of Interest

In some embodiments, the systems described herein comprise a polynucleotide encoding a gene of interest. As used herein, the term “gene of interest” refers to the gene selected for a particular purpose and being desired of delivery by a system or vesicle of the present invention. A gene of interest inserted into one or more regions a vector, such as an expression vector (including one or more of the engineered delivery vesicle generation system vectors) such that when expressed in a target cell or recipient cell it can be expressed and produce a desired gene product and/or be packaged as cargo in an engineered delivery vesicle of the present invention. It will be appreciated that other cargos specifically identified can also be genes of interest. For example, a polynucleotide encoding a Cas effector can be a gene of interest in this context where it is desired to deliver a Cas effector to a cell, for example.

In one embodiment, the gene of interest encodes a gene that provides a therapeutic function for the treatment of a disease. In some embodiments, the gene of interest can also be a vaccinating gene, that is to say a gene encoding an antigenic peptide that is capable of generating an immune response in humans or animals. This may include, but is not necessarily limited to, peptide antigens specific for viral and bacterial infections, or may be tumor-specific. In some embodiments, a gene of interest is a gene which confers a desired phenotype. As the embodiments described herein focus on improved methods for packaging and delivery of a gene of interest, the particular gene of interest is not limiting, and the technology can generally be used to deliver any gene of interest generally recognized by one of ordinary skill in the art as deliverable using a lentiviral system. One skilled in the art can design a construct containing any gene that they are interested in. Designing a construct containing a known gene of interest can be performed without undue experimentation. One of ordinary skill in the art routinely selects genes of interest. For example, the GenBank public database has existed since 1982 and is routinely used by persons of ordinary skill in the art relevant to the presently claimed method. As of June 2019, GenBank contains 2013,383,758 loci, 329,835,282, 370 bases, from 213,383,758 reported sequences. The nucleotide sequences are from more than 300,000 organisms with supporting bibliographic and biological annotation. GenBank is only example, as there are many other known repositories of sequence information.

In some embodiments, the gene of interest may be, for example, a synthetic RNA/DNA sequence, a codon optimized RNA/DNA sequence, a recombinant RNA/DNA sequence (i.e., prepared by use of recombinant DNA techniques), a cDNA sequence or a partial genomic DNA sequence, including combinations thereof. Preferably, this is in the sense orientation. Preferably, the sequence is, comprises, or is transcribed from cDNA. The gene(s) of interest may also be referred to herein as “heterologous sequence(s)” “heterologous gene(s)” or “transgene(s)”.

In some embodiments, the gene of interest may confer some therapeutic benefit. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

Preferably, the therapeutic agent may be administered in a therapeutically effective amount of the active components. The term “therapeutically effective amount” refers to an amount which can elicit a biological or medicinal response in a tissue, system, animal, or human that is being sought by a researcher, veterinarian, medical doctor or other clinician, and in particular can prevent or alleviate one or more of the local or systemic symptoms or features of a disease or condition being treated. In some embodiments, the disease or condition is a disease or condition of or affecting the CNS or cell thereof. Exemplary diseases and disorders of and/or affecting the CNS are described in greater detail elsewhere herein.

In some embodiments, the gene of interest may lead to altered expression in the target cell. As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

Also, “altered expression” as intended herein may encompass modulating the activity of one or more endogenous gene products. Accordingly, “altered expression”, “altering expression”, “modulating expression”, or “detecting expression” or similar may be used interchangeably with respectively “altered expression or activity”, “altering expression or activity”, “modulating expression or activity”, or “detecting expression or activity” or similar. As used herein, “modulating” or “to modulate” generally means either reducing or inhibiting the activity of a target or antigen, or alternatively increasing the activity of the target or antigen, as measured using a suitable in vitro, cellular, or in vivo assay. In particular, “modulating” or “to modulate” can mean either reducing or inhibiting the (relevant or intended) activity of, or alternatively increasing the (relevant or intended) biological activity of the target or antigen, as measured using a suitable in vitro, cellular or in vivo assay (which will usually depend on the target or antigen involved), by at least 5%, at least 10%, at least 25%, at least 50%, at least 60%, at least 70%, at least 80%, or 90% or more, compared to activity of the target or antigen in the same assay under the same conditions but without the presence of the inhibitor/antagonist agents or activator/agonist agents described herein.

As will be clear to the skilled person, “modulating” can also involve effecting a change (which can either be an increase or a decrease) in affinity, avidity, specificity and/or selectivity of a target or antigen, for one or more of its targets compared to the same conditions but without the presence of a modulating agent. Again, this can be determined in any suitable manner and/or using any suitable assay known per se, depending on the target. In particular, an action as an inhibitor/antagonist or activator/agonist can be such that an intended biological or physiological activity is increased or decreased, respectively, by at least 5%, at least 10%, at least 25%, at least 50%, at least 60%, at least 70%, at least 80%, or 90% or more, compared to the biological or physiological activity in the same assay under the same conditions but without the presence of the inhibitor/antagonist agent or activator/agonist agent. Modulating can also involve activating the target or antigen or the mechanism or pathway in which it is involved.

### Interference RNAs

In certain example embodiments, the one or more polynucleotides may encode one or more interference RNAs. Iinterference RNAs are RNA molecules capable of suppressing gene expressions. Example types of interference RNAs include small interfering RNA (siRNA), micro RNA (miRNA), and short hairpin RNA (shRNA).

In certain example embodiments, the interference RNA may be a siRNAs. Small interfering RNA (siRNA) molecules are capable of inhibiting target gene expression by interfering RNA. siRNAs may be chemically synthesized, or may be obtained by in vitro transcription, or may be synthesized in vivo in target cell. siRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode shRNAs. In shRNAs the antiparallel strands that form siRNA are connected by a loop or hairpin region.

The interference RNA (e.g., siRNA) may suppress expression of genes to promote long term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway. TGFβ pathway genes may comprise one or more of ACVR1, ACVR1C, ACVR2A, ACVR2B, ACVRL1, AMH, AMHR2, BMP2, BMP4, BMP5, BMP6, BMP7, BMP8A, BMP8B, BMPR1A, BMPR1B, BMPR2, CDKN2B, CHRD, COMP, CREBBP, CUL1, DCN, E2F4, E2F5, EP300, FST, GDF5, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2R1B, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

In some embodiments, the cargo polynucleotide is an RNAi molecule, antisense molecule, and/or a gene silencing oligonucleotide or a polynucleotide that encodes an RNAi molecule, antisense molecule, and/or gene silencing oligonucleotide.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, and short-hairpin RNA (shRNA). Commercially available programs and tools are available to design the nucleotide sequence of gene silencing oligonucleotides for a desired gene, based on the gene sequence and other information available to one of ordinary skill in the art.

### Therapeutic Polynucleotides

In some embodiments, the cargo molecule is a therapeutic polynucleotide. Therapeutic polynucleotides are those that provide a therapeutic effect when delivered to a recipient cell. The polynucleotide can be a toxic polynucleotide (a polynucleotide that when transcribed or translated results in the death of the cell) or polynucleotide that encodes a lytic peptide or protein. In embodiments, delivery vesicles having a toxic polynucleotide as a cargo molecule can act as an antimicrobial or antibiotic. This is discussed in greater detail elsewhere herein. In some embodiments, the cargo molecule can be exogenous to the producer cell and/or a first cell. In some embodiments, the cargo molecule can be endogenous to the producer cell and/or a first cell. In some embodiments, the cargo molecule can be exogenous to the recipient cell and/or a second cell. In some embodiments, the cargo molecule can be endogenous to the recipient cell and/or second cell.

As described herein the cargo polynucleotide can be any polynucleotide endogenous or exogenous to the eukaryotic cell. For example, the cargo polynucleotide can be a polynucleotide residing in the nucleus of the eukaryotic cell. The cargo polynucleotide can be a sequence coding a gene product (e.g., a protein) or a non-coding sequence (e.g., a regulatory polynucleotide).

In some embodiments, the cargo polynucleotide is a DNA or RNA (e.g., a mRNA) vaccine.

### Aptamers

In certain example embodiments, the polynucleotide may be an aptamer. In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues, and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH2), 2′-fluoro (2′-F), and/or 2′-O-methyl (2′-OMe) substituents. Modifications of aptamers may also include modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to, those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see e.g., SomaLogic, Inc., Boulder, Colorado). In certain embodiments, the present invention may utilize any aptamer containing any modification as described herein.

In certain other example embodiments, the polynucleotide may be a ribozyme or other enzymatically active polynucleotide.

### Biologically Active Agents

In some embodiments, the cargo is a biologically active agent. Biologically active agents include any molecule that induces, directly or indirectly, an effect in a cell. Biologically active agents may be a protein, a nucleic acid, a small molecule, a carbohydrate, and a lipid. When the cargo is or comprises a nucleic acid, the nucleic acid may be a separate entity from the DNA-based carrier. In these embodiments, the DNA-based carrier is not itself the cargo. In other embodiments, the DNA-based carrier may itself comprise a nucleic acid cargo. Therapeutic agents include, without limitation, chemotherapeutic agents, anti-oncogenic agents, anti-angiogenic agents, tumor suppressor agents, anti-microbial agents, enzyme replacement agents, gene expression modulating agents and expression constructs comprising a nucleic acid encoding a therapeutic protein or nucleic acid, and vaccines. Therapeutic agents may be peptides, proteins (including enzymes, antibodies and peptidic hormones), ligands of cytoskeleton, nucleic acid, small molecules, non-peptidic hormones and the like. To increase affinity for the nucleus, agents may be conjugated to a nuclear localization sequence. Nucleic acids that may be delivered by the method of the invention include synthetic and natural nucleic acid material, including DNA, RNA, transposon DNA, antisense nucleic acids, dsRNA, siRNAs, transcription RNA, messenger RNA, ribosomal RNA, small nucleolar RNA, microRNA, ribozymes, plasmids, expression constructs, etc.

Imaging agents include contrast agents, such as ferrofluid-based MRI contrast agents and gadolinium agents for PET scans, fluorescein isothiocyanate and 6-TAMARA. Monitoring agents include reporter probes, biosensors, green fluorescent protein, and the like. Reporter probes include photo-emitting compounds, such as phosphors, radioactive moieties, and fluorescent moieties, such as rare earth chelates (e.g., europium chelates), Texas Red, rhodamine, fluorescein, FITC, fluo-3, 5 hexadecanoyl fluorescein, Cy2, fluor X, Cy3, Cy3.5, Cy5, Cy5.5, Cy7, dansyl, phycocrytherin, phycocyanin, spectrum orange, spectrum green, and/or derivatives of any one or more of the above. Biosensors are molecules that detect and transmit information regarding a physiological change or process, for instance, by detecting the presence or change in the presence of a chemical. The information obtained by the biosensor typically activates a signal that is detected with a transducer. The transducer typically converts the biological response into an electrical signal. Examples of biosensors include enzymes, antibodies, DNA, receptors, and regulator proteins used as recognition elements, which can be used either in whole cells or isolated and used independently (D'Souza, 2001, Biosensors and Bioelectronics 16:337-353).

One or two or more different cargoes may be delivered by the delivery particles described herein.

In some embodiments, the cargo may be linked to one or more envelope proteins by a linker, as described elsewhere herein. A suitable linker may include, but is not necessarily limited to, a glycine-serine linker. In some embodiments, the glycine-serine linker is (GGS)₃.

In some embodiments, the cargo comprises a ribonucleoprotein. In specific embodiments, the cargo comprises a genetic modulating agent.

As used herein the term “altered expression” may particularly denote altered production of the recited gene products by a cell. As used herein, the term “gene product(s)” includes RNA transcribed from a gene (e.g., mRNA), or a polypeptide encoded by a gene or translated from RNA.

### Genetic Modifying Systems

In some embodiments, the cargo is a polynucleotide encoding a gene modifying system. Gene modifying systems may include, but are not limited to, zinc finger nucleases, TALE nucleases (TALENs), meganucleases, RNAi, and CRISPR-Cas systems.

### CRISPR-Cas Systems

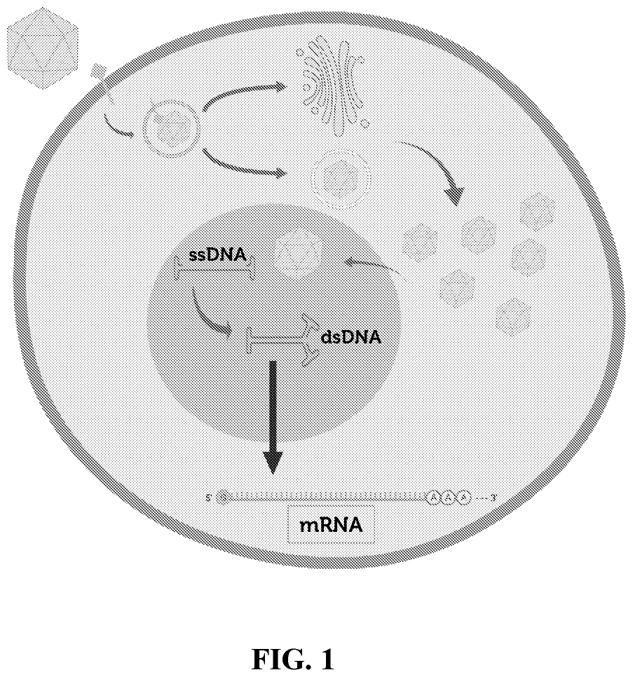

The CRISPR-Cas system may include a Class 1 comprising a Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), and incorporated in its entirety herein by reference, and particularly as described in FIG. 1, p. 326. polynucleotide modifying system or component(s) thereof. The CRISPR-Cas system may also be a Class 2 CRISPR-Cas system such as a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference.

CRISPR-Cas systems may also include further modified systems where the Cas protein is rendered catalytically inactive and fused to other functional domains or polypeptides to derive new functions. Example modified systems include base editor, primer editors, and CRISPR-associated transposase (CAST) systems.

Example base editing systems include DNA base editors (Komor et al. 2016 Nature. 533:420-424; Nishida et a. 2016. Science 353; Gaudelli et al. 2017 Nature 551:464-471; Mok et al., Cell. 182, 463-480 (2020); Koblan et al., Nature 589, 608-614 (2021); Rees and Liu. 2018. 19(12):770-788. doi: 10.1038/s41576-018-0059-1; Song et al., Nat Biomed Eng. 2020 January; 4(1):125-130. doi: 10.1038/s41551-019-0357-8; Koblan et al. 2018. 6(9):843-846. doi: 10.1038/nbt.4172; Thuronyi et al., Nat Biotechnol. 2019 September; 37(9):1070-1079. doi: 10.1038/s41587-019-0193-0; Doman et al., Nat Biotechnol. 2020 May; 38(5):620-628. doi: 10.1038/s41587-020-0414-6; Richter et al., Nat Biotechnol. 2020 July; 38(7):883-891. doi: 10.1038/s41587-020-0453-z; Huang et al., Nat Protoc. 2021 February; 16(2):1089-1128. doi: 10.1038/s41596-020-00450-9; Koblan et al., Nat Biotechnol. 2021 Jun. 28. doi: 10.1038/s41587-021-00938-z; WO 2018/213708, WO 2018/213726, WO/2019/126709, WO/2019/1267; WO/2019/126762) and RNA base editors (Cox et al. 2017. Science 358:1019-1027, Rees and Liu. 2018. 19(12):770-788. doi: 10.1038/s41576-018-0059-1; Abudayyeh 00, et al., A cytosine deaminase for programmable single-base RNA editing, Science 26 Jul. 2019; WO 2019/005883, WO 2019/005886, WO 2019/071048, PCT/US2018/0579, PCT US/2018/067207).

Example prime editing systems include those as described in Anzalone et al. 2019 Nature 576:149-157; Gao et al. 2021 Genome Biol. 22:83; Jang et al. 2021 Nature Biomed. Eng. doi.org/10.1038/s41551-021-00788-9; WO 2021/072328; WO 2020/191248; WO 2020/191249; WO 2020/191239; WO 2020/191245; WO 2020/191246; WO 2020/191241; WO 2020/191171; WO 202/191153; WO 2020/191242; WO 2020/191233; WO 2020/191243; and WO 2020/191234.

Example CAST systems include those as described in Klompe et al. 2019 Nature 571(7764):219-225; Strecker et al. 2019 Science 365:48-53; and Saito et al. 2021 Cell 184:2441-2453; WO 2020/131862; WO 2019090173; WO 2019090174; WO 2019090175, and WO 2019/241452.

Example non-LTR retrotransposon systems include those as described in WO2021/102042.

Example Cas-associated ligase systems include those as described in WO2021/133977.

For modified CRISPR-Cas system that exceed the cargo capacity for a delivery vehicle incorporating the targeting moieties disclosed herein, a split-intein approach to divide CBE and ABE into reconstitutable halves, is described in Levy et al. Nature Biomedical Engineering doi.org/10.1038/s41441-019-0505-5 (2019), which is incorporated herein by reference.

### Zinc Finger Nucleases

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Polypeptides

In certain example embodiments, the cargo molecule may one or more polypeptides. The polypeptide may be a full-length protein or a functional fragment or functional domain thereof, that is a fragment or domain that maintains the desired functionality of the full-length protein. As used within this section “protein” is meant to refer to full-length proteins and functional fragments and domains thereof. A wide array of polypeptides may be delivered using the engineered delivery vesicles described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered delivery vesicles and systems described herein via delivery of the corresponding encoding polynucleotide.

### Secretory Proteins

In certain example embodiments, the one or more polypeptides may comprise one or more secretory proteins. A secretory is a protein that is actively transported out of the cell, for example, the protein, whether it be endocrine or exocrine, is secreted by a cell. Secretory pathways have been shown conserved from yeast to mammals, and both conventional and unconventional protein secretion pathways have been demonstrated in plants. Chung et al., “An Overview of Protein Secretion in Plant Cells,” MIMB, 1662:19-32, Sep. 1, 2017. Accordingly, identification of secretory proteins in which one or more polynucleotides may be inserted can be identified for particular cells and applications. In embodiments, one of skill in the art can identify secretory proteins based on the presence of a signal peptide, which consists of a short hydrophobic N-terminal sequence.

In embodiments, the protein is secreted by the secretory pathway. In embodiments, the proteins are exocrine secretion proteins or peptides, comprising enzymes in the digestive tract. In embodiments the protein is endocrine secretion protein or peptide, for example, insulin and other hormones released into the blood stream. In other embodiments, the protein is involved in signaling between or within cells via secreted signaling molecules, for example, paracrine, autocrine, endocrine or neuroendocrine. In embodiments, the secretory protein is selected from the group of cytokines, kinases, hormones and growth factors that bind to receptors on the surface of target cells.

As described, secretory proteins include hormones, enzymes, toxins, and antimicrobial peptides. Examples of secretory proteins include serine proteases (e.g., pepsins, trypsin, chymotrypsin, elastase and plasminogen activators), amylases, lipases, nucleases (e.g. deoxyribonucleases and ribonucleases), peptidases enzyme inhibitors such as serpins (e.g., al-antitrypsin and plasminogen activator inhibitors), cell attachment proteins such as collagen, fibronectin and laminin, hormones and growth factors such as insulin, growth hormone, prolactin platelet-derived growth factor, epidermal growth factor, fibroblast growth factors, interleukins, interferons, apolipoproteins, and carrier proteins such as transferrin and albumins. In some examples, the secretory protein is insulin or a fragment thereof. In one example, the secretory protein is a precursor of insulin or a fragment thereof. In certain examples, the secretory protein is c-peptide. In a preferred embodiment, the one or more polynucleotides is inserted in the middle of the c-peptide. In some aspects, the secretory protein is GLP-1, glucagon, betatrophin, pancreatic amylase, pancreatic lipase, carboxypeptidase, secretin, CCK, a PPAR (e.g. PPAR-alpha, PPAR-gamma, PPAR-delta or a precursor thereof (e.g. preprotein or preproprotein). In aspects, the secretory protein is fibronectin, a clotting factor protein (e.g. Factor VII, VIII, IX, etc.), a2-macroglobulin, al-antitrypsin, antithrombin III, protein S, protein C, plasminogen, a2-antiplasmin, complement components (e.g. complement component C1-9), albumin, ceruloplasmin, transcortin, haptoglobin, hemopexin, IGF binding protein, retinol binding protein, transferrin, vitamin-D binding protein, transthyretin, IGF-1, thrombopoietin, hepcidin, angiotensinogen, or a precursor protein thereof. In aspects, the secretory protein is pepsinogen, gastric lipase, sucrase, gastrin, lactase, maltase, peptidase, or a precursor thereof. In aspects, the secretory protein is renin, erythropoietin, angiotensin, adrenocorticotropic hormone (ACTH), amylin, atrial natriuretic peptide (ANP), calcitonin, ghrelin, growth hormone (GH), leptin, melanocyte-stimulating hormone (MSH), oxytocin, prolactin, follicle-stimulating hormone (FSH), thyroid stimulating hormone (TSH), thyrotropin-releasing hormone (TRH), vasopressin, vasoactive intestinal peptide, or a precursor thereof.

### Immunomodulatory Polypeptides

In certain example embodiments, the one or more polypeptides may comprise one or more immunomodulatory protein. In certain embodiments, the present invention provides for modulating immune states. The immune state can be modulated by modulating T cell function or dysfunction. In particular embodiments, the immune state is modulated by expression and secretion of IL-10 and/or other cytokines as described elsewhere herein. In certain embodiments, T cells can affect the overall immune state, such as other immune cells in proximity.

The polynucleotides may encode one or more immunomodulatory proteins, including immunosuppressive proteins. The term “immunosuppressive” means that immune response in an organism is reduced or depressed. An immunosuppressive protein may suppress, reduce, or mask the immune system or degree of response of the subject being treated. For example, an immunosuppressive protein may suppress cytokine production, downregulate or suppress self-antigen expression, or mask the MHC antigens. As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some cases, the immunosuppressive proteins may exert pleiotropic functions. In some cases, the immunomodulatory proteins may maintain proper regulatory T cells versus effector T cells (Treg/Teff) balance. For examples, the immunomodulatory proteins may expand and/or activate the Tregs and blocks the actions of Teffs, thus providing immunoregulation without global immunosuppression. Target genes associated with immune suppression include, for example, checkpoint inhibitors such PD1, Tim3, Lag3, TIGIT, CTLA-4, and combinations thereof.

The term “immune cell” as used throughout this specification generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, Thαβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naive B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

T cell response refers more specifically to an immune response in which T cells directly or indirectly mediate or otherwise contribute to an immune response in a subject. T cell-mediated response may be associated with cell mediated effects, cytokine mediated effects, and even effects associated with B cells if the B cells are stimulated, for example, by cytokines secreted by T cells. By means of an example but without limitation, effector functions of MHC class I restricted Cytotoxic T lymphocytes (CTLs), may include cytokine and/or cytolytic capabilities, such as lysis of target cells presenting an antigen peptide recognized by the T cell receptor (naturally-occurring TCR or genetically engineered TCR, e.g., chimeric antigen receptor, CAR), secretion of cytokines, preferably IFN gamma, TNF alpha and/or or more immunostimulatory cytokines, such as IL-2, and/or antigen peptide-induced secretion of cytotoxic effector molecules, such as granzymes, perforins or granulysin. By means of example but without limitation, for MHC class II restricted T helper (Th) cells, effector functions may be antigen peptide-induced secretion of cytokines, preferably, IFN gamma, TNF alpha, IL-4, IL5, IL-10, and/or IL-2. By means of example but without limitation, for T regulatory (Treg) cells, effector functions may be antigen peptide-induced secretion of cytokines, preferably, IL-10, IL-35, and/or TGF-beta. B cell response refers more specifically to an immune response in which B cells directly or indirectly mediate or otherwise contribute to an immune response in a subject. Effector functions of B cells may include in particular production and secretion of antigen-specific antibodies by B cells (e.g., polyclonal B cell response to a plurality of the epitopes of an antigen (antigen-specific antibody response)), antigen presentation, and/or cytokine secretion.

During persistent immune activation, such as during uncontrolled tumor growth or chronic infections, subpopulations of immune cells, particularly of CD8+ or CD4+ T cells, become compromised to different extents with respect to their cytokine and/or cytolytic capabilities. Such immune cells, particularly CD8+ or CD4+ T cells, are commonly referred to as “dysfunctional” or as “functionally exhausted” or “exhausted”. As used herein, the term “dysfunctional” or “functional exhaustion” refer to a state of a cell where the cell does not perform its usual function or activity in response to normal input signals, and includes refractivity of immune cells to stimulation, such as stimulation via an activating receptor or a cytokine. Such a function or activity includes, but is not limited to, proliferation (e.g., in response to a cytokine, such as IFN-gamma) or cell division, entrance into the cell cycle, cytokine production, cytotoxicity, migration and trafficking, phagocytotic activity, or any combination thereof. Normal input signals can include, but are not limited to, stimulation via a receptor (e.g., T cell receptor, B cell receptor, co-stimulatory receptor). Unresponsive immune cells can have a reduction of at least 10%, 20%, 30%, 40%, 50%, 60%, 70%, 80%, 90%, 95%, or even 100% in cytotoxic activity, cytokine production, proliferation, trafficking, phagocytotic activity, or any combination thereof, relative to a corresponding control immune cell of the same type. In some particular embodiments of the aspects described herein, a cell that is dysfunctional is a CD8+ T cell that expresses the CD8+ cell surface marker. Such CD8+ cells normally proliferate and produce cell killing enzymes, e.g., they can release the cytotoxins perforin, granzymes, and granulysin. However, exhausted/dysfunctional T cells do not respond adequately to TCR stimulation, and display poor effector function, sustained expression of inhibitory receptors and a transcriptional state distinct from that of functional effector or memory T cells. Dysfunction/exhaustion of T cells thus prevents optimal control of infection and tumors. Exhausted/dysfunctional immune cells, such as T cells, such as CD8+ T cells, may produce reduced amounts of IFN-gamma, TNF-alpha and/or one or more immunostimulatory cytokines, such as IL-2, compared to functional immune cells. Exhausted/dysfunctional immune cells, such as T cells, such as CD8+ T cells, may further produce (increased amounts of) one or more immunosuppressive transcription factors or cytokines, such as IL-10 and/or Foxp3, compared to functional immune cells, thereby contributing to local immunosuppression. Dysfunctional CD8+ T cells can be both protective and detrimental against disease control. As used herein, a “dysfunctional immune state” refers to an overall suppressive immune state in a subject or microenvironment of the subject (e.g., tumor microenvironment). For example, increased IL-10 production leads to suppression of other immune cells in a population of immune cells.

CD8+ T cell function is associated with their cytokine profiles. It has been reported that effector CD8+ T cells with the ability to simultaneously produce multiple cytokines (polyfunctional CD8+ T cells) are associated with protective immunity in patients with controlled chronic viral infections as well as cancer patients responsive to immune therapy (Spranger et al., 2014, J. Immunother. Cancer, vol. 2, 3). In the presence of persistent antigen CD8+ T cells were found to have lost cytolytic activity completely overtime (Moskophidis et al., 1993, Nature, vol. 362, 758-761). It was subsequently found that dysfunctional T cells can differentially produce IL-2, TNFa and IFNg in a hierarchical order (Wherry et al., 2003, J. Virol., vol. 77, 4911-4927). Decoupled dysfunctional and activated CD8+ cell states have also been described (see, e.g., Singer, et al. (2016). A Distinct Gene Module for Dysfunction Uncoupled from Activation in Tumor-Infiltrating T Cells. Cell 166, 1500-1511 e1509; WO/2017/075478; and WO/2018/049025).

The invention provides compositions and methods for modulating T cell balance. The invention provides T cell modulating agents that modulate T cell balance. For example, in some embodiments, the invention provides T cell modulating agents and methods of using these T cell modulating agents to regulate, influence or otherwise impact the level of and/or balance between T cell types, e.g., between Th17 and other T cell types, for example, Th1-like cells. For example, in some embodiments, the invention provides T cell modulating agents and methods of using these T cell modulating agents to regulate, influence or otherwise impact the level of and/or balance between Th17 activity and inflammatory potential. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3.

In some examples, immunomodulatory proteins may be immunosuppressive cytokines. In general, cytokines are small proteins and include interleukins, lymphokines and cell signal molecules, such as tumor necrosis factor and the interferons, which regulate inflammation, hematopoiesis, and response to infections. Examples of immunosuppressive cytokines include interleukin 10 (IL-10), TGF-P, IL-Ra, IL-18Ra, IL-2, IL-3, IL-4, IL-5, IL-6, IL-7, IL-8, IL-9, IL-11, IL-12, IL-13, IL-14, IL-15, IL-16, IL-17, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, PGE2, SCF, G-CSF, CSF-1R, M-CSF, GM-CSF, IFN-α, IFN-β, IFN-γ, IFN-4, bFGF, CCL2, CXCL1, CXCL8, CXCL12, CX3CL1, CXCR4, TNF-α and VEGF. Examples of immunosuppressive proteins may further include FOXP3, AHR, TRP53, IKZF3, IRF4, IRF1, and SMAD3. In one example, the immunosuppressive protein is IL-10. In one example, the immunosuppressive protein is IL-6. In one example, the immunosuppressive protein is IL-2.

### Anti-Fibrotic Proteins

In certain example embodiments, the one or more polypeptides may comprise an anti-fibrotic protein. Examples of anti-fibrotic proteins include any protein that reduces or inhibits the production of extracellular matrix components, fibronectin, proteoglycan, collagen, elastin, TGIFs, and SMAD7. In embodiments, the anti-fibrotic protein is a peroxisome proliferator-activated receptor (PPAR) or may include one or more PPARs. In some embodiments, the protein is PPARα, PPAR γ is a dual PPARα/γ. Derosa et al., “The role of various peroxisome proliferator-activated receptors and their ligands in clinical practice” Jan. 18, 2017 J. Cell. Phys. 223:1 153-161.

Proteins that Promote Tissue Regeneration and or Transplant Survival Functions

In certain example embodiments, the one or more polypeptides may comprise proteins that promote tissue regeneration and/or transplant survival functions. In some cases, such proteins may induce and/or up-regulate the expression of genes for pancreatic β cell regeneration. In some cases, the proteins that promote transplant survival and functions include the products of genes for pancreatic R cell regeneration. Such genes may include proislet peptides that are proteins or peptides derived from such proteins that stimulate islet cell neogenesis. Examples of genes for pancreatic R cell regeneration include Reg1, Reg2, Reg3, Reg4, human proislet peptide, parathyroid hormone-related peptide (1-36), glucagon-like peptide-1 (GLP-1), extendin-4, prolactin, Hgf, Igf-1, Gip-1, adipsin, resistin, leptin, IL-6, IL-10, Pdx1, Ptfal, Mafa, Pax6, Pax4, Nkx6.1, Nkx2.2, PDGF, vglycin, placental lactogens (somatomammotropins, e.g., CSH1, CHS2), isoforms thereof, homologs thereof, and orthologs thereof. In certain embodiments, the protein promoting pancreatic B cell regeneration is a cytokine, myokine, and/or adipokine.

### Hormones

In certain embodiments, the one or mor polynucleotides may comprise one or more hormones. The term “hormone” refers to polypeptide hormones, which are generally secreted by glandular organs with ducts. Hormones include proteins from natural sources or from recombinant cell culture and biologically active equivalents of the native sequence hormone, including synthetically produced small-molecule entities and pharmaceutically acceptable derivatives and salts thereof. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); prolactin, placental lactogen, mouse gonadotropin-associated peptide, inhibin; activin; mullerian-inhibiting substance; and thrombopoietin, growth hormone (GH), adrenocorticotropic hormone (ACTH), dehydroepiandrosterone (DHEA), cortisol, epinephrine, thyroid hormone, estrogen, progesterone, placental lactogens (somatomammotropins, e.g. CSH1, CHS2), testosterone. and neuroendocrine hormones. In certain examples, the hormone is secreted from pancreas, e.g., insulin, glucagon, somatostatin, pancreatic polypeptide and ghrelin. In some examples, the hormone is insulin.

Hormones herein may also include growth factors, e.g., fibroblast growth factor (FGF) family, bone morphogenic protein (BMP) family, platelet derived growth factor (PDGF) family, transforming growth factor beta (TGFbeta) family, nerve growth factor (NGF) family, epidermal growth factor (EGF) family, insulin related growth factor (IGF) family, hepatocyte growth factor (HGF) family, hematopoietic growth factors (HeGFs), platelet-derived endothelial cell growth factor (PD-ECGF), angiopoietin, vascular endothelial growth factor (VEGF) family, and glucocorticoids. In a particular embodiment, the hormone is insulin or incretins such as exenatide, GLP-1.

### Neurohormones

In embodiments, the secreted peptide is a neurohormone, a hormone produced and released by neuroendocrine cells. Example neurohormones include Thyrotropin-releasing hormone, Corticotropin-releasing hormone, Histamine, Growth hormone-releasing hormone, Somatostatin, Gonadotropin-releasing hormone, Serotonin, Dopamine, Neurotensin, Oxytocin, Vasopressin, Epinephrine, and Norepinephrine.

### Anti-Microbial Proteins

In some embodiments, the one or more polypeptides may comprise one or more anti-microbial proteins. In embodiments where the cell is mammalian cell, human host defense antimicrobial peptides and proteins (AMPs) play a critical role in warding off invading microbial pathogens. In certain embodiments, the anti-microbial is α-defensin HD-6, HNP-1 and β-defensin hBD-3, lysozyme, cathelcidin LL-37, C-type lectin RegIIIalpha, for example. See, e.g., Wang, “Human Antimicrobial Peptide and Proteins” Pharma, May 2014, 7(5): 545-594, incorporated herein by reference.

### Anti-Fibrillating Proteins

In certain example embodiments, the one or more polypeptides may comprise one or more anti-fibrillating polypeptides. The anti-fibrillating polypeptide can be the secreted polypeptide. In some embodiments, the anti-fibrillating polypeptide is co-expressed with one or more other polynucleotides and/or polypeptides described elsewhere herein. The anti-fibrillating agent can be secreted and act to inhibit the fibrillation and/or aggregation of endogenous proteins and/or exogenous proteins that it may be co-expressed therewith. In some embodiments, the anti-fibrillating agent is P4 (VITYF (SEQ ID NO: 1700)), P5 (VVVVV (SEQ ID NO: 1701)), KR7 (KPWWPRR (SEQ ID NO: 1702)), NK9 (NIVNVSLVK (SEQ ID NO: 1703)), iAb5p (Leu-Pro-Phe-Phe-Asp (SEQ ID NO: 1704)), KLVF (SEQ ID NO: 1705) and derivatives thereof, indolicidin, camosine, a hexapeptide as set forth in Wang et al. 2014. ACS Chem Neurosci. 5:972-981, alpha sheet peptides having alternating D-amino acids and L-amino acids as set forth in Hopping et al. 2014. Elife 3:e01681, D-(PGKLVYA) (SEQ ID NO: 1706), RI-OR2-TAT, cyclo(17, 21)-(Lysl7, Asp21)A_(1-28), SEN304, SEN1576, D3, R8-Aβ(25-35), human yD-crystallin (HGD), poly-lysine, heparin, poly-Asp, polyGl, poly-L-lysine, poly-L-glutamic acid, LVEALYL (SEQ ID NO: 1707), RGFFYT (SEQ ID NO: 1708), a peptide set forth or as designed/generated by the method set forth in U.S. Pat. No. 8,754,034, and combinations thereof. In aspects, the anti-fibrillating agent is a D-peptide. In aspects, the anti-fibrillating agent is an L-peptide. In aspects, the anti-fibrillating agent is a retro-inverso modified peptide. Retro-inverso modified peptides are derived from peptides by substituting the L-amino acids for their D-counterparts and reversing the sequence to mimic the original peptide since they retain the same spatial positioning of the side chains and 3D structure. In aspects, the retro-inverso modified peptide is derived from a natural or synthetic Ap peptide. In some embodiments, the polynucleotide encodes a fibrillation resistant protein. In some embodiments, the fibrillation resistant protein is a modified insulin, see e.g., U.S. Pat. No. 8,343,914.

### Antibodies

In certain embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

### Protease Cleavage Sites

The one or more cargo polypeptides, as exemplified above, may comprise one or more protease cleavage sites, i.e., amino acid sequences that can be recognized and cleaved by a protease. The protease cleavage sites may be used for generating desired gene products (e.g., intact gene products without any tags or portion of other proteins). The protease cleavage site may be one end or both ends of the protein. Examples of protease cleavage sites that can be used herein include an enterokinase cleavage site, a thrombin cleavage site, a Factor Xa cleavage site, a human rhinovirus 3C protease cleavage site, a tobacco etch virus (TEV) protease cleavage site, a dipeptidyl aminopeptidase cleavage site and a small ubiquitin-like modifier (SUMO)/ubiquitin-like protein-1(ULP-1) protease cleavage site. In certain examples, the protease cleavage site comprises Lys-Arg.

### Small Molecules

In some embodiments, the cargo molecule is a small molecule. Techniques and methods of coupling peptides to small molecule agents are generally known in the art and can be applied here to couple a targeting moiety effective to target a CNS cell to a small molecule cargo. Small molecules include, without limitation, hormones, immunomodulators, antipyretics, anxiolytics, antipsychotics, analgesics, antispasmodics, anti-inflammatories, anti-histamines, anti-infectives, radiation sensitizers, chemotherapeutics.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosteron Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-α, IFN-β, IFN-ε, IFN-K, IFN-Ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g., ibuprofen, naproxen, ketoprofen, and nimesulide), aspirin and related salicylates (e.g., choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene. Suitable anti-inflammatories include, but are not limited to, prednisone, non-steroidal anti-inflammants (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, abendazole, thiabendazole, oxamniquine), antifungals (e.g., azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g., vancomycin, dalbavancin, oritavancin, and telvancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g., lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfasoxazole), tetracyclines (e.g., doxycycline, demeclocycline, minocycline, doxycycline/salicyclic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

### Muscle Disease Cargos

In some embodiments, the cargo molecule can be a polynucleotide or polypeptide that can alone or when delivered as part of a system, whether or not delivered with other components of the system, operate to modify the genome, epigenome, and/or transcriptome of a cell to which it is delivered, is such that it treats or prevents a disease, a disorder, or a symptom thereof of a muscle disease, disorder, or a symptom thereof, a CNS disease, disorder and/or a symptom thereof, or both. In some embodiments, the cargo molecule, whether or not delivered with other components of the system, operate to modify the genome, epigenome, and/or transcriptome of a cell to which it is delivered, is such that it treats or prevents a disease, a disorder, or a symptom thereof of a muscle disease, disorder, or a symptom thereof, a CNS disease, disorder and/or a symptom thereof, or both. In some embodiments, the disease or disorder is a progeroid disease, (e.g., progeroid laminopathy) a glycogen storage disease an immune disorder (such as an autoimmune disease), a cancer, Duchenne muscular dystrophy (DMD), 6 Limb-girdle muscular dystrophy diseases (LGMD), Charcot-Marie-Tooth (CMT), MPS IIIA, Pompe disease, or other CNS-related diseases such as Huntington's and other expanded repeat diseases.

In some embodiments, the cargo molecule, whether or not delivered with other components of the system, operate to modify the genome, epigenome, and/or transcriptome of a cell to which it is delivered, is such that can modify the GAA gene, such as any of those described in US Pat. App. Pub. 20190284555, the contents of which are incorporated by reference as if expressed in their entirety herein and can be adapted for use with the present invention.

In some embodiments, the cargo molecule includes an oligonucleotide coupled to a MHCK7, CK8, or other muscle specific promoter.

In some embodiments, the cargo molecule is a micro-dystrophin oligonucleotide that contains only selected regions of the dystrophin gene optimized for protein functionality. In some embodiments, the selected regions include spectrin-like repeats 1, 2, 3, and 24. See e.g., Harper S Q, Hauser M A, DelloRusso C, et al. Modular flexibility of dystrophin: implications for gene therapy of Duchenne muscular dystrophy. Nat Med. 2002; 8(3):253-261. In some embodiments, the micro-dystrophin oligonucleotide is that is delivered by the rAAV agent known as AAVrh74.MHCK7 microdystrophin gene or SRP-9001, which is subject to the clinical trials NCT03375164 and NCT03769116. This microdystrophin gene construct includes NT-H1-R1-R2-R3-H2-R24-H4-CR-CT. In some embodiments, the microdystrophin gene includes ABD-H1-R1-R2-R3-H2-R24-H4-CR-CT. In some embodiments, the microdystrophin gene includes H stands for hinge region. England S B, et al. Nature. 1990; 343(6254):180-182; Wells D J, et al. Hum Mol Genet. 1995; 4(8):1245-1250, Salva M Z, et al. Mol Ther. 2007; 15(2):320-329; Mendell J R, et al. Neurosci Lett. 2012; 527(2):90-99; Rodino-Klapac L R, et al. Hum Mol Genet. 2013; 22(24):4929-4937; Velazquez V M, et al. Mol Ther Methods Clin Dev. 2017; 4:159-168; Harper S Q, et al. Nat Med. 2002; 8(3):253-261; Nelson D M, et al. Hum Mol Genet. 2018; 27(12):2090-2100. In some embodiments, the selected regions at least include spectrin-like repeats 2 and 3. In some embodiments, the micro-dystrophin gene contains a nNOS domain. In some embodiments, the nNOS domain is composed of spectrin-like repeats 16 and/or 17. In some embodiments, the micro-dystrophin gene includes spectrin-like repeats 16 and 17. In some embodiments, the nNOS domain is composed of spectrin-like repeats RI, R16, R17, R23, and R24. In some embodiments, the micro-dystrophin gene is coupled to a muscle specific promoter. In some embodiments, the micro-dystrophin oligonucleotide is coupled to a MHCK7, CK8, SNP18, SP0033, SP0051, SP0173, tmCK, or another muscle specific promoter.

In some embodiments, the cargo micro-dystrophin includes an ABD (actin binding domain), one or more hinge regions (e.g., H1, H2, H3, H4,), and one or more spectrin-like repeats (e.g., R1, R1′ R2, R3, R16, R17, R20, R21, R22, R23, R24, R24′ and optionally a dystroglycan binding domain (DBD). In some embodiments, the micro-dystrophin is composed of ABD-H1-R1-R16-R17-R23-R24-H4-DBD. In some embodiments, the micro-dystrophin is composed of ABD-H1-R1-R2-R3-H2-R24-H4-CR. In some embodiments, the micro-dystrophin gene includes ABD-H1-R1-R2-R3-H2-R24-H4-CR-CT. In some embodiments, the micro-dystrophin gene includes ABD-H1-R1′-R24′-H4-CR-CT.

In some embodiments, the cargo molecule is a polynucleotide that can encode a micro-dystrophin gene, where the micro-dystrophin gene contains spectrin-like repeats, R1, R16, R17, R23 and R24. In some embodiments, the micro-dystrophin gene contains hinge region (H) 4 and/or H1. In some embodiments, the micro-dystrophin gene contains the N-terminal actin binding domain. In some embodiments, the micro-dystrophin gene contains the C-terminal dystroglycan binding domain of the human full-length dystrophin protein. The micro-dystrophin gene can contain an nNOS domain. In some embodiments, the nNOS domain is composed of spectrin-like repeats 16 and/or 17. In some embodiments, the micro-dystrophin gene includes spectrin-like repeats 16 and 17. The micro-dystrophin gene can be as described in WO2019118806A1 and WO2016/115543, which are incorporated by reference as if expressed in their entirety herein and can be adapted for use with the present invention. In some embodiments, the cargo polynucleotide can encode a 5-repeat micro-dystrophin protein that contains, from N- to C-terminus, the N-terminal actin binding domain, Hinge region 1 (HI), spectrin-like repeats RI, R16, R17, R23, and R24, Hinge region 4 (H4), and the C-terminal dystroglycan binding domain of the human full-length dystrophin protein. The protein sequence of this 5-repeat micro-dystrophin and the related dystrophin minigene are described in WO2016/115543. In some embodiments, the cargo polynucleotide can correspond to a micro-dystrophin gene that is part of the agent known as SGT001 as currently in clinical trial having the identifier number NCT03368742.

In some embodiments, the cargo molecule is a minidys gene or vector. In some embodiments, the minidys gene or vector can be composed of ABD-H1-R1-R2-R3-R16-R17-H3-R20-R21; ABD-H1-RI-R2-R3-R16-R17-H3-R20-R21-R22-R23-R24-H4-CR; or H3-R20-R21-R22-R23-R24-H4-CR-CT.

In some embodiments, the cargo molecule is an SCGB cDNA. In some embodiments, the SGCB cDNA is coupled to a MHCK7, CK8 promoter, SNP18 promoter, SP0033 promoter, SP0051, SP0173 promoter, tmCK promoter or another muscle specific promoter. In some embodiments, the cargo molecule is a beta-sarcoglycan cDNA, an alpha-sarcoglycan cDNA, a dysferlin cDNA, a gamma-sarcoglycan cDNA, a Calpin-3 cDNA, a SGSH cDNA (e.g., LYS-SAF302), a neurtropin 3 cDNA, an anoctamin-5 cDNA, or any combination thereof.

In some embodiments, the cargo molecule, whether or not delivered with other components of the system, operate to modify the genome, epigenome, and/or transcriptome of a cell to which it is delivered, is such that treat, prevent, and/or modify a gene or gene product associated with an expanded repeat disease, such as Huntington's disease, such as those described in U.S. Pat. App. Pub. 20190100755, U.S. patent Ser. No. 10/066,228, the contents of which are incorporated by reference as if expressed in their entirety herein and can be adapted for use with the present invention.

In some embodiments, the cargo molecule is an antisense oligomer or RNA molecule, such as those described in U.S. Pat. App. Pub. US20160251398, US20150267202, US20190015440, US20140287983, US20180216111, WO/2017/062835, US20190177723, US20170051278, US20180271893, WO/2016/14965, U.S. patent Ser. No. 10/076,536, WO/2018/00580, WO/2018/11866, WO/2019/059973, the contents of which are incorporated by reference as if expressed in their entirety herein and can be adapted for use with the present invention.

In some embodiments, the cargo molecule, whether or not delivered with other components of the system, operate to modify the genome, epigenome, and/or transcriptome of a cell to which it is delivered, is such that it treats or prevents a single stranded RNA virus, such influenza, West Nile Virus, SARS, Hepatitis C, dengue fever, Ebola, Marburg, and/or Calicivirus. In some embodiments the cargo molecule can be an antisense antiviral compound, such as any of those described in U.S. Pat. No. 8,703,735B2, the contents of which are incorporated by reference as if expressed in their entirety herein and can be adapted for use with the present invention.

Additional, exemplary genetic and gene associated diseases and genes capable of being modified by a cargo molecule described herein are listed elsewhere herein, see e.g., Tables 12-13.

In some embodiments, the cargo molecule can add or modify a GALGT2 gene. Instead of acting to resupply missing dystrophin, GALGT2 gene therapy fortifies the structural integrity of muscle in ways that compensate for the absence of dystrophin, by increasing expression of proteins not mutated or lost in the disease. GALGT2 offers the potential to treat DMD irrespective of specific dystrophin mutation, as well as having utility in other muscular dystrophies.

In some embodiments the cargo molecule is a morpholino, such as in US Patent Application Pub. US2018/0161359 and US2019/0054113 the contents of which are incorporated by reference as if expressed in their entirety herein and can be adapted for use with the present invention. In some embodiments, the morpholino is a morpholino oligomer (PMO) or a peptide linked morpholino PPMO. PMO based platforms can be used to treat genetic diseases by altering mRNA transcription. PMOs are synthetic chemical structures modeled after the natural framework of RNA. While PMOs have the same nucleic acid bases found in RNA, they are bound to six-sided morpholine rings instead of five-sided ribose rings. In addition, the morpholine rings are connected to each other by phosphorodiamidate linkages instead of the phosphodiester linkages found in RNA. PMOs and PPMOs can be used for exon skipping and translation suppression.

In some embodiments, the cargo molecule can be a peptide-oligomer, conjugate as described in e.g., International Patent Application Publication WO2017106304A 1, the contents of which are incorporated by reference as if expressed in their entirety herein and can be adapted for use with the present invention.

In some embodiments, the morpholino is the morpholino found in Eteplirsen, which can be effective to target Exon 51 of the dystrophin mRNA. In some embodiments the cargo molecule can generate exon skipping in the context of DMD, such as those described in e.g., US Patent Application Pub. US2014/0315977A1 and US2018/010581, the contents of which are incorporated by reference as if expressed in their entirety herein and can be adapted for use with the present invention.

### Exon Skipping

In some embodiments, the nucleotide sequences may encode nucleic acids capable of inducing exon skipping. Such encoded nucleic acids may be antisense oligonucleotides or antisense nucleotide systems. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using antisense oligonucleotides (AON) that are complementary to splice donor sequences within a pre-mRNA.

In some embodiments, the nucleotide sequences encode antisense oligonucleotides or antisense nucleotide systems capable of inducing exon skipping in dystrophin mRNA. For example, a non-sense or frameshift mutation within exon x of a dystrophin gene yields a carboxy-terminally truncated, non-functional dystrophin protein. The expression of that mature mRNA transcript may yield a functional dystrophin protein that is deleted in the amino acids encoded by exon x but that includes dystrophin amino acids both N-terminal and C-terminal to those deleted amino acids.

The nucleotide sequences may encode antisense oligonucleotides or antisense nucleotide systems capable of inducing exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof. The nucleotide sequences may encode antisense oligonucleotides or antisense nucleotide systems capable of inducing exon skipping at exon 43, 44, 50, 51, 52, 55, or any combination thereof.

### Engineered Viral Capsids and Encoding Polynucleotides

Described herein are exemplary embodiments of engineered viral proteins, (e.g., capsid proteins), such as adeno-associated virus (AAV) viral proteins (e.g., capsid proteins), that can be engineered to confer cell-specific tropism to an engineered viral particle (AAV particle) that contains the engineered viral protein(s). The engineered viral protein(s) (e.g., capsid(s)) can be included in an engineered virus particle, and can confer cell-specific tropism, such as both a CNS and muscle specific tropism or a muscle specific tropism, reduced immunogenicity, or both to the engineered viral (e.g., an AAV) particle. As is described elsewhere herein, the particles can include a cargo. In this way, the particles can be a cell-specific delivery vehicle for a cargo. The engineered viral capsids described herein can include one or more engineered viral capsid proteins described herein. Engineered viral capsid proteins can be lentiviral, retroviral, adenoviral, or AAV. Engineered capsids can contain one or more of the viral capsid proteins. Engineered virus particles can include one or more of the engineered viral capsid proteins and thus contain an engineered viral capsid. The engineered viral capsid proteins, capsids, and/or viral particles that contain one or more CNS-muscle specific or muscle specific targeting moieties containing or composed of one or more n-mer insert s described elsewhere herein. In some embodiments, the engineered viral capsid proteins, viral capsids, and/or viral particles can have both a CNS and muscle specific tropism or a muscle tropism (e.g., a cardiac muscle specific tropism and/or a skeletal muscle specific tropism) conferred to it by the one or more n-mer inserts contained therein.

The CNS-muscle specific or muscle specific n-mer inserts and targeting moieties can be encoded in whole or in part by a polynucleotide. The engineered viral capsid and/or viral capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

The engineered AAV capsid can contain 1-60 engineered capsid proteins. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV capsid can contain 0-59 wild-type AAV capsid proteins. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins.

In some embodiments, the engineered AAV capsid protein can have an n-mer amino acid insert (also referred to herein as an “n-mer insert”), where n can be at least 3 amino acids. In some embodiments, n can be 3, 4, 5, 6, 7, 8, 9, 10, 11, 12, 13, 14, 15, 16, 17, 18, 19, 20, 21, 22, 23, 24, or 25 amino acids. In some embodiments, the engineered AAV capsid can have a 6-mer or 7-mer amino acid insert. In some embodiments, the n-mer amino acid inset can be inserted between two amino acids in the wild-type viral protein (VP) (or capsid protein). In some embodiments, the n-mer insert can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parovirus capsids (see e.g., DiMattia et al. 2012. J. Virol. 86(12):6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In some embodiments, one or more n-mer inserts can be inserted between two amino acids in one or more of the 12 variable regions in the wild-type AVV capsid proteins. In some embodiments, the one or more n-mer inserts can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer insert(s) can be inserted between two amino acids in the VR-III of a capsid protein. In some embodiments, the engineered capsid can have an n-mer insert inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral protein. In some embodiments, the engineered capsid can have an n-mer insert inserted between amino acids 588 and 589 of an AAV9 viral protein. In some embodiments, the engineered capsid can have an n-mer insert inserted between amino acids 588 and 589 of an AAV9 viral protein. In some embodiments, the engineered capsid can have an n-mer insert inserted between amino acids 588 and 589 of an AAV9 viral protein. SEQ ID NO: 1 is a reference AAV9 capsid sequence for at least referencing the insertion sites discussed above. It will be appreciated that n-mer insert(s) can be inserted in analogous positions in AAV viral proteins of other serotypes. In some embodiments as previously discussed, the n-mer insert(s) can be inserted between any two contiguous amino acids within the AAV viral protein and in some embodiments the insertion is made in a variable region.

In certain example embodiments, one or more of the n-mer motifs are incorporated into the viral protein such that at least one of the one or more RGD motifs, at least one of the one or more P motifs, or both is/are located between two amino acids of the viral protein such that at least one of the one or more RGD motifs and/or one or more P-motifs is external to a viral capsid (e.g., an AAV viral capsid).

In certain example embodiments, one or more of the one or more n-mer inserts are incorporated into the AAV protein such that one or more of the one more RGD motifs and/or one or more of the one or more P motifs are each inserted between any two contiguous amino acids between amino acids independently selected from 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

In certain example embodiments, at least one of the one or more n-mer inserts is incorporated into the AAV protein such that at least one of the one more RGD motifs and/or at least one of the one or more P motifs is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

In certain example embodiments, at least one of the one or more n-mer inserts is incorporated into the AAV protein such that at least one of the one more RGD motifs and/or at least one of the one or more P motifs is inserted between amino acids 598-599 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

AAV9 capsid (wild-type) reference Sequence:
SEQ ID NO: 1
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL

In some embodiments, an AAV capsid can contain one or more targeting moieties having one or more n-mer inserts that contain a P-motif, an RGD motif or both. P-motifs and RGD motifs are described in greater detail elsewhere herein. In some embodiments, an AAV capsid can contain one or more targeting moieties having one or more n-mer inserts that are each immediately preceded by AQ or DG. In some embodiments, the n-mer insert in an AAV capsid, such as an CNS-muscle specific or muscle specific AAV capsid, can be or include one or more RGD motifs and/or P motifs as in any one or more as set forth in Tables 4, 5, 6, 7, 8, 9, 10, 11, or any combination thereof. In some embodiments, insertion of the n-mer insert in an AAV capsid can result in cell, tissue, organ, specific engineered AAV capsids. In some embodiments, the engineered viral protein, engineered viral capsid protein, engineered viral capsid, and/or engineered viral particle can have a specificity for muscle cells (e.g., cardiac and/or skeletal muscle cells). In some embodiments, the engineered viral protein, engineered viral capsid protein, engineered viral capsid, and/or engineered viral particle can have a specificity for both muscle cells and CNS cells and/or tissue. In some embodiments, the engineered viral protein, engineered viral capsid protein, engineered viral capsid, and/or engineered viral particle can have a specificity for both cardiac muscle cells and CNS cells and/or tissue. In some embodiments, the engineered viral protein, engineered viral capsid protein, engineered viral capsid, and/or engineered viral particle can have a specificity for both skeletal muscle cells and CNS cells and/or tissue. In some embodiments, the engineered viral protein, engineered viral capsid protein, engineered viral capsid, and/or engineered viral particle can have a specificity for muscle cells and/or tissue. In some embodiments, the engineered viral protein, engineered viral capsid protein, engineered viral capsid, and/or engineered viral particle can have a specificity for cardiac muscle cells and/or tissue. In some embodiments, the engineered viral protein, engineered viral capsid protein, engineered viral capsid, and/or engineered viral particle can have a specificity for skeletal muscle cells and/or tissue.

In some embodiments, the CNS-muscle specific or muscle specific AAV capsid contains an RGD insert and/or a P-motif. In some embodiments, the CNS-muscle specific or muscle specific AAV capsid does not contain an RGD insert. In some embodiments, the CNS-muscle specific or muscle specific AAV capsid does not contain a P-motif P-motifs and RGD motifs are described in greater detail elsewhere herein.

In some embodiments, the n-mer insert(s) include one or more “P motifs” and/or one or more RGD motifs. In some embodiments, the P-motif comprises or consists of the amino acid sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7. In some embodiments, the P-motif has the amino acid sequence PX₁QGTX₂RXₙ (SEQ ID NO: 1699), where X₁, X₂, Xₙ, are each selected from any amino acid and where n is 0, 1, 2, 3, 4, 5, 6, or 7. Exemplary P-motifs are described in greater detail elsewhere herein. In some embodiments, at least one of the RGD motifs comprises or consists of XₘRGDXₙ, wherein m is 0-4 amino acids, wherein n is 0-15 amino acids, and wherein Xₘ, and X₁ are each independently selected from any amino acid. In some embodiments, X₁ is S, T, or A. In some embodiments, X₂ is L, V, F, or I. In some embodiments, X₁ is S, T, or A and X₂ is L, V, F, or I. Exemplary, non-limiting P motifs are shown at least in e.g., Tables 8, 10, 9, and/or 11. Exemplary, non-limiting P motifs are shown at least in e.g., Tables 8, 10, 9, and/or 11. Exemplary, non-limiting P motifs are shown at least in e.g., Tables 4-11 or any combination thereof. Exemplary non-limiting RGD motifs are shown in at least Tables 4, 5, 6, 7, 10, and/or 11. In some embodiments, one or more n-mer inserts that can be or include a P-motif and/or RGD motif as set forth in any one or more of Tables 4, 5, 6, 7, 8, 9, 10, 11, or any combination thereof can be included in a CNS-muscle specific or a muscle specific engineered capsid.

In some embodiments, the n-mer insert (such as a 7-mer insert) can be inserted into an AAV vector between two contiguous amino acids where the amino acids in the AAV vector immediately preceding the n-mer insert can be DG or AQ. In some embodiments, the DG or AQ are the amino acids immediately preceding the n-mer insert in the capsid protein when the n-mer insert is included in a capsid polypeptide, particularly an AAV capsid polypeptide. Without being bound by theory, inserts including a DG or AQ at the C terminal end or are inserted into a capsid polypeptide, such as an AAV capsid polypeptide, such that the insert(s) are immediately following an AQ or DG of the capsid polypeptide, may be able to transduce more hosts, such as more strains or species. In some embodiments, amino acids 587 and 588 of the AAV or analogous amino acids thereto are DG. In some embodiments, amino acids 587 and 588 of the AAV or analogous amino acids thereto are AQ. In some embodiments, amino acids 587 and 588 of the AAV or analogous amino acids thereto are AQ and are followed by an n-mer insert. In some embodiments, amino acids 587 and 588 of the AAV or analogous amino acids thereto are DG and are followed by an n-mer insert.

In some embodiments, the n-mer insert is such that when included in a host polypeptide (e.g., viral or AAV protein, such as a capsid protein) one or more residues of the host polypeptide are replaced with one or more of that from the n-mer insert. In some embodiments, when a C terminal AQ or DG are included in the n-mer insert but are not part of a P or RGD motif, the AQ or DG can optionally replace 1 or 2 amino acid residues immediately preceding where the P or RGD motif is to be inserted. For example, in some embodiments, where the RGD or P motif is desired to be inserted between e.g., 588 and 589 in an AAV9 or position analogous thereto in other AAVs, the n-mer insert can contain e.g., [AQ or DG]-[P or RGD motif]-Xₙ or Xₘ, where Xₙ or Xₘ is as described elsewhere herein with respect to the P and RGD motifs, respectively, where AQ or DG replaces residues 587 and 588 of the AAV9 or position analogous thereto in other AAVs leaving the P or RGD motif to be effectively inserted between positions 588 and 589 of the AAV9 or position analogous thereto in other AAVs. It will be appreciated that such an approach can be extrapolated to other host polypeptides besides AAVs as well as other positions within AAVs. Further this can be extrapolated to other C-terminal amino acids besides AQ or DG as the case may be (e.g., Xₘ in the context of P motifs).

In some embodiments, the AAV or other viral capsids are both CNS and muscle specific. In some embodiments, the AAV or other viral capsids are muscle specific. In some embodiments, the AAV or other viral capsids is skeletal muscle specific and/or cardiac muscle specific. In some embodiments, the muscle (e.g., skeletal and/or cardiac muscle) specificity of the engineered AAV or other viral capsid is conferred by a muscle (e.g., skeletal and/or cardiac muscle) specific n-mer insert incorporated in the engineered AAV or other viral capsid. In some embodiments, the dual CNS-muscle (e.g., CNS and muscle) specificity of the engineered AAV or other viral capsid is conferred by a CNS-muscle specific n-mer insert incorporated in the engineered AAV or other viral capsid. While not intending to be bound by theory, it is believed that the n-mer insert confers a 3D structure to or within a domain or region of the engineered AAV or other viral capsid such that the interaction of an engineered AAV containing said engineered AAV or other viral capsid has increased or improved interactions (e.g., increased affinity) with a cell surface receptor and/or other molecule on the surface of a muscle cell (e.g., a cardiac cell and/or skeletal muscle cell) or both a CNS cell and a muscle cell (e.g., a cardiac muscle and/or skeletal muscle cell). In some embodiments the cell surface receptor is AAV receptor (AAVR). In some embodiments, the cell surface receptor is a muscle (e.g., a cardiac muscle cell and/or skeletal muscle cell) cell specific AAV receptor. In some embodiments, the cell surface receptor is a CNS cell and a muscle (e.g., a cardiac muscle cell and/or skeletal muscle cell) cell specific AAV receptor. In some embodiments, a CNS-muscle specific or a muscle specific (e.g. a cardiac muscle and/or skeletal muscle) engineered AAV containing the CNS-muscle specific or muscle (e.g., cardiac and/or skeletal) specific capsid can have an increased transduction rate, efficiency, amount, or a combination thereof in a CNS and/or a muscle (e.g., a cardiac or skeletal muscle) cell as compared to other cell types and/or other AAVs or other viruses that do not contain a muscle-specific engineered AAV or other viral capsid as described herein.

Also described herein are polynucleotides that encode the engineered targeting moieties, viral proteins (e.g., capsid proteins), and other polypeptides described herein, including but not limited to, the engineered AAV or other viral capsids described herein. In some embodiments, the engineered AAV or other viral capsid encoding polynucleotide can be included in a polynucleotide that is configured to be an AAV or other viral genome donor in an AAV or other viral vector system that can be used to generate engineered AAV or other viral particles described elsewhere herein.

In some embodiments the engineered viral (e.g., AAV) capsid encoding polynucleotide can be operably coupled to a poly adenylation tail. In some embodiments, the poly adenylation tail can be an SV40 poly adenylation tail. In some embodiments, the viral (e.g., AAV) capsid encoding polynucleotide can be operably coupled to a promoter. In some embodiments, the promoter can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.) and combinations thereof. In some embodiments the promoter can be a constitutive promoter. Suitable tissue specific promoters and constitutive promoters are discussed elsewhere herein and are generally known in the art and can be commercially available.

Suitable muscle tissue/cell specific promoters include, but are not limited to CK8, MHCK7, Myoglobin promoter (Mb), Desmin promoter, muscle creatine kinase promoter (MCK) and variants thereof, and SPc5-12 synthetic promoter.

Suitable neuronal tissue/cell specific promoters include, but are not limited to, GFAP promoter (astrocytes), SYN1 promoter (neurons), and NSE/RU5′ (mature neurons).

Other suitable CNS specific promoters can include, but are not limited to, neuroactive peptide cholecystokinin (CCK) (see e.g., Chhatawl et al. Gene Therapy volume 14, pages575-583(2007)), a brain specific DNA MiniPromoter (such as any of those identified for brain or pan-neronal expression as in de Leeuw et al. Mol. Therapy. 1(5): 2014. doi:10.1038/mtm.2013.5), myelin basic promoter (MBP) (see e.g., von Jonquieres, G., Mersmann, N., Klugmann, C. B., Harasta, A. E., Lutz, B., Teahan, O., et al. (2013). Glial promoter selectivity following AAV-delivery to the immature brain. PLoS One 8 (6), e65646. doi: 10.1371/journal.pone.0065646), glial fibrillary acid protein (GFAP) for expression in astrocytes (see e.g., Smith-Arica, J. R., Morelli, A. E., Larregina, A. T., Smith, J., Lowenstein, P. R., Castro, M. G. (2000). Cell-type-specific and regulatable transgenesis in the adult brain: adenovirus-encoded combined transcriptional targeting and inducible transgene expression. Mol. Ther. 2 (6), 579-587. doi: 10.1006/mthe.2000.0215 and Lee, Y., Messing, A., Su, M., Brenner, M. (2008). GFAP promoter elements required for region-specific and astrocyte-specific expression. Glia 56 (5), 481-493. doi: 10.1002/glia.20622), human myelin associated glycoprotein promoter (full-length or truncated) (see e.g., von Jonquieres, G., Frohlich, D., Klugmann, C. B., Wen, X., Harasta, A. E., Ramkumar, R., et al. (2016). Recombinant human myelin-associated glycoprotein promoter drives selective AAV-mediated transgene expression in oligodendrocytes. Front. Mol. Neurosci. 9, 13. doi: 10.3389/fnmol.2016.00013), F4/80 promoter (see e.g., Rosario, A. M., Cruz, P. E., Ceballos-Diaz, C., Strickland, M. R., Siemienski, Z., Pardo, M., et al. (2016). Microglia-specific targeting by novel capsid-modified AAV6 vectors. Mol. Ther. Methods Clin. Dev. 3, 16026. doi: 10.1038/mtm.2016.26), phosphate-activated glutaminase (PAG) or the vesicular glutamate transporter (vGLUT) promoter (for about 90% glutamatergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), glutamic acid decarboxylase (GAD) promoter (for about 90% GABAergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), MeCP2 promoter (see e.g., Gray et al. Hum Gene Ther. 2011 September; 22(9):1143-53. doi: 10.1089/hum.2010.245), and retinoblastoma gene promoter (see e.g., Jiang et al., J. Biol. Chem. 2001. 276, 593-600).

Suitable constitutive promoters include, but are not limited to CMV, RSV, SV40, EF1alpha, CAG, and beta-actin.

AAVs with Reduced Non-CNS and/or Non-Muscle Cell Specificity

In some embodiments, the n-mer insert(s), RGD motifs, and/or P-motif(s) are inserted into an AAV protein (e.g., an AAV capsid protein) that has reduced specificity (or no detectable, measurable, or clinically relevant interaction) for one or more non-CNS cell and/or non-muscle (e.g., skeletal muscle and/or cardiac muscle) cell types. Exemplary non-CNS and/or non-muscle (e.g., skeletal muscle and/or cardiac muscle) cell types include, but are not limited to, liver, kidney, lung, heart, spleen, muscle (skeletal and cardiac), bone, immune, stomach, intestine, eye, skin cells and the like. In some embodiments, the non-CNS and/or non-muscle (e.g., skeletal muscle and/or cardiac muscle) cells are liver cells.

In certain example embodiments, the AAV capsid protein is an engineered AAV capsid protein having reduced or eliminated uptake in a non-CNS and/or non-muscle (e.g., skeletal muscle and/or cardiac muscle) cell as compared to a corresponding wild-type AAV capsid polypeptide.

In certain example embodiments, the non-CNS and/or non-muscle (e.g., skeletal muscle and/or cardiac muscle) cell is a liver cell.

In certain example embodiments, the wild-type capsid polypeptide is an AAV1, AAV2, AAV3, AAV4, AAV5, AAV6, AAV7, AAV8, AAV9, AAV rh.74, or AAV rh.10 capsid polypeptide.

In certain example embodiments, the engineered AAV capsid protein comprises one or more mutations that result in reduced or eliminated uptake in a non-CNS and/or non-muscle (e.g., skeletal muscle and/or cardiac muscle) cell.

In certain example embodiments, the one or more mutations are in position 267, in position 269, in position 504, in position 505, in position 590, or any combination thereof in the AAV9 capsid protein (SEQ ID NO: 1) or in one or more positions corresponding thereto in a non-AAV9 capsid polypeptide.

In certain example embodiments, the non-AAV9 capsid protein is an AAV1, AAV2, AAV3, AAV4, AAV5, AAV6, AAV7, AAV8, AAV rh.74, or AAV rh.10 capsid polypeptide.

In certain example embodiments, the mutation in position 267 in the AAV9 capsid protein (SEQ ID NO: 1) or position corresponding thereto in a non-AAV9 capsid polypeptide is a G or X mutation to A, wherein X is any amino acid.

In certain example embodiments, the mutation in position 269 in the AAV9 capsid protein (SEQ ID NO: 1) or position corresponding thereto in a non-AAV9 capsid polypeptide is an S or X to T mutation, wherein X is any amino acid.

In certain example embodiments, the mutation in position 504 in the AAV9 capsid protein (SEQ ID NO: 1) or position corresponding thereto in a non-AAV9 capsid polypeptide is a G or X to A mutation, wherein X is any amino acid.

In certain example embodiments, the mutation in position 505 in the AAV9 capsid protein (SEQ ID NO: 1) or position corresponding thereto in a non-AAV9 capsid polypeptide is a P or X to A mutation, wherein X is any amino acid.

In certain example embodiments, the mutation in position 590 in the AAV9 capsid protein (SEQ ID NO: 1) or position corresponding thereto in a non-AAV9 capsid polypeptide is a Q or X to A mutation, wherein X is any amino acid.

In certain example embodiments, the engineered AAV capsid protein is an engineered AAV9 capsid polypeptide comprising a mutation at position 267, position 269 or both of a wild-type AAV9 capsid protein (SEQ ID NO: 1), wherein the mutation at position 267 is a G to A mutation and wherein the mutation at position 269 is an S to T mutation.

In certain example embodiments, the engineered AAV capsid protein is an engineered AAV9 capsid polypeptide comprising a mutation at position 590 of a wild-type AAV9 capsid protein (SEQ ID NO: 1), wherein the mutation at position 509 is a Q to A mutation.

In certain example embodiments, the engineered AAV capsid protein is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

In some embodiments, the AAV capsid protein in which the n-mer insert(s) and/or P motif(s) can be inserted can be 80-100 (e.g., 80, 81, 82, 83, 84, 85, 86, 87, 88, 89, 90, 91, 92, 93, 94, 95, 96, 97, 98, 99, to/or 100) percent identical to SEQ ID NO: 4 or SEQ ID NO: 5 of International Patent Application Publication WO 2019/217911, which is incorporated by reference as if expressed in its entirety herein. These sequences are also incorporated herein as SEQ ID NOS: 2 and 3 respectively. It will be appreciated that when considering variants of these AAV9 capsid proteins with reduced liver specificity, that residues 267 and/or 269 must contain the relevant mutations or equivalents.

AAV9_G267A
SEQ ID NO: 2:
Met Ala Ala Asp Gly Tyr Leu Pro Asp Trp Leu Glu Asp Asn Leu Ser
Glu Gly Ile Arg Glu Trp Trp Ala Leu Lys Pro Gly Ala Pro Gln Pro
Lys Ala Asn Gln Gln His Gln Asp Asn Ala Arg Gly Leu Val Leu Pro
Gly Tyr Lys Val Leu Gly Pro Gly Asn Gly Leu Asp Lys Gly Glu Pro
Val Asn Ala Ala Asp Ala Ala Ala Leu Glu His Asp Lys Ala Tyr Asp
Gln Gln Leu Lys Ala Gly Asp Asn Pro Tyr Leu Lys Tyr Asn His Ala
Asp Ala Glu Phe Gln Glu Arg Leu Lys Glu Asp Thr Ser Phe Gly Gly
Asn Leu Gly Arg Ala Val Phe Gln Ala Lys Lys Arg Leu Leu Glu Pro
Leu Gly Leu Val Glu Glu Ala Ala Lys Thr Ala Pro Gly Lys Lys Arg
Pro Val Glu Gln Ser Pro Gln Glu Pro Asp Ser Ser Ala Gly Ile Gly
Lys Ser Gly Ala Gln Pro Ala Lys Lys Arg Leu Asn Phe Gly Gln Thr
Gly Asp Thr Glu Ser Val Pro Asp Pro Gln Pro Ile Gly Glu Pro Pro
Ala Ala Pro Ser Gly Val Gly Ser Leu Thr Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Ala Ser Ser Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Gln Ala Gln
Thr Gly Trp Val Gln Asn Gln Gly Ile Leu Pro Gly Met Val Trp Gln
Asp Arg Asp Val Tyr Leu Gln Gly Pro Ile Trp Ala Lys Ile Pro His
Thr Asp Gly Asn Phe His Pro Ser Pro Leu Met Gly Gly Phe Gly Met
Lys His Pro Pro Pro Gln Ile Leu Ile Lys Asn Thr Pro Val Pro Ala
Asp Pro Pro Thr Ala Phe Asn Lys Asp Lys Leu Asn Ser Phe Ile Thr
Gln Tyr Ser Thr Gly Gln Val Ser Val Glu Ile Glu Trp Glu Leu Gln
Lys Glu Asn Ser Lys Arg Trp Asn Pro Glu Ile Gln Tyr Thr Ser Asn
Tyr Tyr Lys Ser Asn Asn Val Glu Phe Ala Val Asn Thr Glu Gly Val
Tyr Ser Glu Pro Arg Pro Ile Gly Thr Arg Tyr Leu Thr Arg Asn Leu
AAV9_G267A, S269T
SEQ ID NO: 3:
Met Ala Ala Asp Gly Tyr Leu Pro Asp Trp Leu Glu Asp Asn Leu Ser
Glu Gly Ile Arg Glu Trp Trp Ala Leu Lys Pro Gly Ala Pro Gln Pro
Lys Ala Asn Gln Gln His Gln Asp Asn Ala Arg Gly Leu Val Leu Pro
Gly Tyr Lys Val Leu Gly Pro Gly Asn Gly Leu Asp Lys Gly Glu Pro
Val Asn Ala Ala Asp Ala Ala Ala Leu Glu His Asp Lys Ala Tyr Asp
Gln Gln Leu Lys Ala Gly Asp Asn Pro Tyr Leu Lys Tyr Asn His Ala
Asp Ala Glu Phe Gln Glu Arg Leu Lys Glu Asp Thr Ser Phe Gly Gly
Asn Leu Gly Arg Ala Val Phe Gln Ala Lys Lys Arg Leu Leu Glu Pro
Leu Gly Leu Val Glu Glu Ala Ala Lys Thr Ala Pro Gly Lys Lys Arg
Pro Val Glu Gln Ser Pro Gln Glu Pro Asp Ser Ser Ala Gly Ile Gly
Lys Ser Gly Ala Gln Pro Ala Lys Lys Arg Leu Asn Phe Gly Gln Thr
Gly Asp Thr Glu Ser Val Pro Asp Pro Gln Pro Ile Gly Glu Pro Pro
Ala Ala Pro Ser Gly Val Gly Ser Leu Thr Met Ala Ser Gly Gly Gly
Ala Pro Val Ala Asp Asn Asn Glu Gly Ala Asp Gly Val Gly Ser Ser
Ser Gly Asn Trp His Cys Asp Ser Gln Trp Leu Gly Asp Arg Val Ile
Thr Thr Ser Thr Arg Thr Trp Ala Leu Pro Thr Tyr Asn Asn His Leu
Tyr Lys Gln Ile Ser Asn Ser Thr Ser Gly Ala Ser Thr Asn Asp Asn
Ala Tyr Phe Gly Tyr Ser Thr Pro Trp Gly Tyr Phe Asp Phe Asn Arg
Phe His Cys His Phe Ser Pro Arg Asp Trp Gln Arg Leu Ile Asn Asn
Asn Trp Gly Phe Arg Pro Lys Arg Leu Asn Phe Lys Leu Phe Asn Ile
Gln Val Lys Glu Val Thr Asp Asn Asn Gly Val Lys Thr Ile Ala Asn
Asn Leu Thr Ser Thr Val Gln Val Phe Thr Asp Ser Asp Tyr Gln Leu
Pro Tyr Val Leu Gly Ser Ala His Glu Gly Cys Leu Pro Pro Phe Pro
Ala Asp Val Phe Met Ile Pro Gln Tyr Gly Tyr Leu Thr Leu Asn Asp
Gly Ser Gln Ala Val Gly Arg Ser Ser Phe Tyr Cys Leu Glu Tyr Phe
Pro Ser Gln Met Leu Arg Thr Gly Asn Asn Phe Gln Phe Ser Tyr Glu
Phe Glu Asn Val Pro Phe His Ser Ser Tyr Ala His Ser Gln Ser Leu
Asp Arg Leu Met Asn Pro Leu Ile Asp Gln Tyr Leu Tyr Tyr Leu Ser
Lys Thr Ile Asn Gly Ser Gly Gln Asn Gln Gln Thr Leu Lys Phe Ser
Val Ala Gly Pro Ser Asn Met Ala Val Gln Gly Arg Asn Tyr Ile Pro
Gly Pro Ser Tyr Arg Gln Gln Arg Val Ser Thr Thr Val Thr Gln Asn
Asn Asn Ser Glu Phe Ala Trp Pro Gly Ala Ser Ser Trp Ala Leu Asn
Gly Arg Asn Ser Leu Met Asn Pro Gly Pro Ala Met Ala Ser His Lys
Glu Gly Glu Asp Arg Phe Phe Pro Leu Ser Gly Ser Leu Ile Phe Gly
Lys Gln Gly Thr Gly Arg Asp Asn Val Asp Ala Asp Lys Val Met Ile
Thr Asn Glu Glu Glu Ile Lys Thr Thr Asn Pro Val Ala Thr Glu Ser
Tyr Gly Gln Val Ala Thr Asn His Gln Ser Ala Gln Ala Gln Ala Gln
Thr Gly Trp Val Gln Asn Gln Gly Ile Leu Pro Gly Met Val Trp Gln
Asp Arg Asp Val Tyr Leu Gln Gly Pro Ile Trp Ala Lys Ile Pro His
Thr Asp Gly Asn Phe His Pro Ser Pro Leu Met Gly Gly Phe Gly Met
Lys His Pro Pro Pro Gln Ile Leu Ile Lys Asn Thr Pro Val Pro Ala
Asp Pro Pro Thr Ala Phe Asn Lys Asp Lys Leu Asn Ser Phe Ile Thr
Gln Tyr Ser Thr Gly Gln Val Ser Val Glu Ile Glu Trp Glu Leu Gln
Lys Glu Asn Ser Lys Arg Trp Asn Pro Glu Ile Gln Tyr Thr Ser Asn
Tyr Tyr Lys Ser Asn Asn Val Glu Phe Ala Val Asn Thr Glu Gly Val
Tyr Ser Glu Pro Arg Pro Ile Gly Thr Arg Tyr Leu Thr Arg Asn Leu

In some embodiments, the AAV capsid protein in which the in which the n-mer insert(s) can be inserted can be 80-100 (e.g., 80, 81, 82, 83, 84, 85, 86, 87, 88, 89, 90, 91, 92, 93, 94, 95, 96, 97, 98, 99, to/or 100) percent identical to any of those described in Adachi et al., (Nat. Comm. 2014. 5:3075, DOI: 10.1038/ncomms4075) that have reduced specificity for a non-CNS cell, particularly a liver cell. Adachi et al., (Nat. Comm. 2014. 5:3075, DOI: 10.1038/ncomms4075) is incorporated by reference herein as if expressed in its entirety.

In some embodiments, the modified AAV can have about a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent or fold reduction in specificity for a non-CNS cells as compared to a wild-type AAV or control. In some embodiments, the modified AAV can have no measurable or detectable uptake and/or expression in one or more non-CNS cells.

### Methods of Generating Engineered AAV Capsids

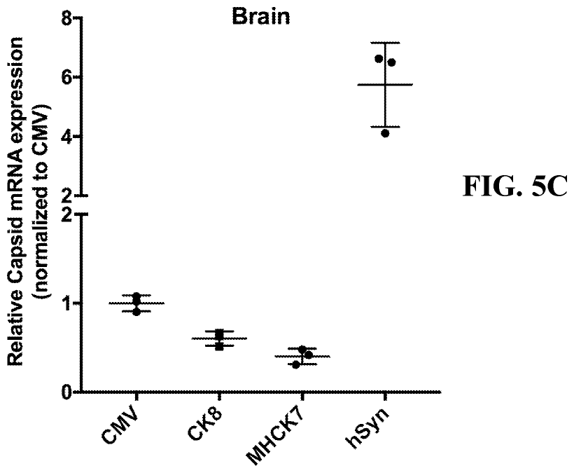

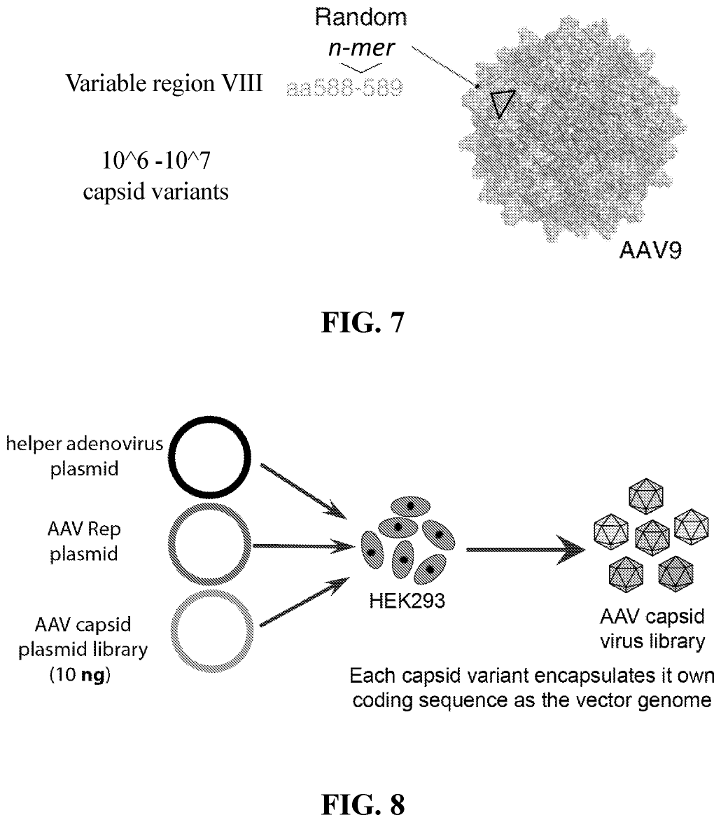

Also provided herein are methods of generating engineered AAV capsids. The engineered AAV capsid variants can be variants of wild-type AAV capsids. FIGS. 6A-8 can illustrate various embodiments of methods capable of generating engineered AAV capsids described herein. Generally, an AAV capsid library can be generated by expressing engineered capsid vectors each containing an engineered AAV capsid polynucleotide previously described in an appropriate AAV producer cell line. See e.g., FIG. 8. It will be appreciated that although FIG. 8 shows a helper-dependent method of AAV particle production, it will be appreciated that this can be done via a helper-free method as well. This can generate an AAV capsid library that can contain one more desired cell-specific engineered AAV capsid variant. As shown in FIG. 6 the AAV capsid library can be administered to various non-human animals for a first round of mRNA-based selection. As shown in FIG. 1, the transduction process by AAVs and related vectors can result in the production of an mRNA molecule that is reflective of the genome of the virus that transduced the cell. As is at least demonstrated in the Examples herein, mRNA based-selection can be more specific and effective to determine a virus particle capable of functionally transducing a cell because it is based on the functional product produced as opposed to just detecting the presence of a virus particle in the cell by measuring the presence of viral DNA.

After first-round administration, one or more engineered AAV virus particles having a desired capsid variant can then be used to form a filtered AAV capsid library. Desirable AAV virus particles can be identified by measuring the mRNA expression of the capsid variants and determining which variants are highly expressed in the desired cell type(s) as compared to non-desired cells type(s). Those that are highly expressed in the desired cell, tissue, and/or organ type are the desired AAV capsid variant particles. In some embodiments, the AAV capsid variant encoding polynucleotide is under control of a tissue-specific promoter that has selective activity in the desired cell, tissue, or organ.

The engineered AAV capsid variant particles identified from the first round can then be administered to various non-human animals. In some embodiments, the animals used in the second round of selection and identification are not the same as those animals used for first round selection and identification. Similar to round 1, after administration the top expressing variants in the desired cell, tissue, and/or organ type(s) can be identified by measuring viral mRNA expression in the cells. The top variants identified after round two can then be optionally barcoded and optionally pooled. In some embodiments, top variants from the second round can then be administered to a non-human primate to identify the top cell-specific variant(s), particularly if the end use for the top variant is in humans. Administration at each round can be systemic.

In some embodiments, the method of generating an AAV capsid variant can include the steps of: (a) expressing a vector system described herein that contains an engineered AAV capsid polynucleotide in a cell to produce engineered AAV virus particle capsid variants; (b) harvesting the engineered AAV virus particle capsid variants produced in step (a); (c) administering engineered AAV virus particle capsid variants to one or more first subjects, wherein the engineered AAV virus particle capsid variants are produced by expressing an engineered AAV capsid variant vector or system thereof in a cell and harvesting the engineered AAV virus particle capsid variants produced by the cell; and (d) identifying one or more engineered AAV capsid variants produced at a significantly high level by one or more specific cells or specific cell types in the one or more first subjects. In this context, “significantly high” can refer to a titer that can range from between about 2×10¹¹ to about 6×10¹² vector genomes per 15 cm dish.

The method can further include the steps of: (e) administering some or all engineered AAV virus particle capsid variants identified in step (d) to one or more second subjects; and (f) identifying one or more engineered AAV virus particle capsid variants produced at a significantly high level in one or more specific cells or specific cell types in the one or more second subjects. The cell in step (a) can be a prokaryotic cell or a eukaryotic cell. In some embodiments, the administration in step (c), step (e), or both is systemic. In some embodiments, one or more first subjects, one or more second subjects, or both, are non-human mammals. In some embodiments, one or more first subjects, one or more second subjects, or both, are each independently selected from the group consisting of: a wild-type non-human mammal, a humanized non-human mammal, a disease-specific non-human mammal model, and a non-human primate.

### Engineered Vectors and Vector Systems

Also provided herein are vectors and vector systems that can contain one or more of the engineered polynucleotides, (e.g., an AAV capsid polynucleotide) described herein. As used in this context, engineered viral (e.g., AAV) capsid polynucleotides refers to any one or more of the polynucleotides described herein capable of encoding an engineered viral (e.g., AAV) capsid as described elsewhere herein and/or polynucleotide(s) capable of encoding one or more engineered viral (e.g., AAV) capsid proteins described elsewhere herein. Further, where the vector includes an engineered viral (e.g., AAV) capsid polynucleotide described herein, the vector can also be referred to and considered an engineered vector or system thereof although not specifically noted as such. In embodiments, the vector can contain one or more polynucleotides encoding one or more elements of an engineered viral (e.g., AAV) capsid described herein. The vectors can be useful in producing bacterial, fungal, yeast, plant cells, animal cells, and transgenic animals that can express one or more components of the engineered viral (e.g., AAV) capsid described herein. Within the scope of this disclosure are vectors containing one or more of the polynucleotide sequences described herein. One or more of the polynucleotides that are part of the engineered viral (e.g., AAV) capsid and system thereof described herein can be included in a vector or vector system.

In some embodiments, the vector can include an engineered viral (e.g., AAV) capsid polynucleotide having a 3′ polyadenylation signal. In some embodiments, the 3′ polyadenylation is an SV40 polyadenylation signal. In some embodiments the vector does not have splice regulatory elements. In some embodiments, the vector includes one or more minimal splice regulatory elements. In some embodiments, the vector can further include a modified splice regulatory element, wherein the modification inactivates the splice regulatory element. In some embodiments, the modified splice regulatory element is a polynucleotide sequence sufficient to induce splicing, between a rep protein polynucleotide and the engineered viral (e.g., AAV) capsid protein variant polynucleotide. In some embodiments, the polynucleotide sequence can be sufficient to induce splicing is a splice acceptor or a splice donor. In some embodiments, the viral (e.g., AAV) capsid polynucleotide is an engineered viral (e.g., AAV) capsid polynucleotide as described elsewhere herein.

The vectors and/or vector systems can be used, for example, to express one or more of the engineered viral (e.g., AAV) capsid polynucleotides in a cell, such as a producer cell, to produce engineered viral (e.g., AAV) particles containing an engineered viral (e.g., AAV) capsid described elsewhere herein. Other uses for the vectors and vector systems described herein are also within the scope of this disclosure. In general, and throughout this specification, the term is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include adeno-associated viruses, and types of such vectors can also be selected for targeting particular types of cells, such as those engineered viral (e.g., AAV) vectors containing an engineered viral (e.g., AAV) capsid polynucleotide with a desired cell-specific tropism. These and other embodiments of the vectors and vector systems are described elsewhere herein.

In some embodiments, the vector can be a bicistronic vector. In some embodiments, a bicistronic vector can be used for one or more elements of the engineered viral (e.g., AAV) capsid system described herein. In some embodiments, expression of elements of the engineered viral (e.g., AAV) capsid system described herein can be driven by a suitable constitutive or tissue specific promoter. Where the element of the engineered viral (e.g., AAV) capsid system is an RNA, its expression can be driven by a Pol III promoter, such as a U6 promoter. In some embodiments, the two are combined.

### Cell-Based Vector Amplification and Expression

Vectors can be designed for expression of one or more elements of the engineered targeting moieties, polypeptides, viral (e.g., AAV) capsid system described herein (e.g., nucleic acid transcripts, proteins, enzymes, and combinations thereof), etc. in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMIBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of an engineered targeting moiety, polypeptide, viral (e.g., AAV) capsid system so as to drive expression of the one or more elements of the engineered targeting moiety, polypeptide, viral (e.g., AAV) capsid system described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET IId (Studier et al., GENE EXPRESSION TECHNOLOGY: METHODS IN ENZYMOLOGY 185, Academic Press, San Diego, Calif (1990) 60-89).

In some embodiments, one or more vectors driving expression of one or more elements of an engineered targeting moiety, polypeptide, viral (e.g., AAV) capsid system described herein are introduced into a host cell such that expression of the elements of the engineered delivery system described herein direct formation of an engineered targeting moiety, polypeptide, viral (e.g., AAV) capsid system described herein (including but not limited to an engineered gene transfer agent particle, which is described in greater detail elsewhere herein). For example, different elements of the engineered targeting moiety, polypeptide, viral (e.g., AAV) capsid system described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the engineered targeting moiety, polypeptide, viral (e.g., AAV) capsid system described herein that incorporates one or more elements of the engineered targeting moiety, polypeptide, viral (e.g., AAV) capsid system described herein or contains one or more cells that incorporates and/or expresses one or more elements of the engineered targeting moiety, polypeptide, viral (e.g., AAV) capsid system described herein.

In some embodiments, two or more of the elements expressed from the same or different regulatory element(s), can be combined in a single vector, with one or more additional vectors providing any components of the system not included in the first vector. Engineered targeting moiety, polypeptide, viral (e.g., AAV) capsid system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered targeting moiety, polypeptide, viral (e.g., AAV) capsid proteins, embedded within one or more intron sequences (e.g., each in a different intron, two or more in at least one intron, or all in a single intron). In some embodiments, the engineered targeting moiety, polypeptide, viral (e.g., AAV) capsid polynucleotides can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (such as the engineered targeting moiety, polypeptide, viral (e.g., AAV) capsid polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. In some embodiments, the regulated promoter is a tissue specific promoter as previously discussed elsewhere herein. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, CNS and/or muscle tissue and cell specific promoters.

Suitable muscle tissue/cell specific promoters include, but are not limited to CK8, MHCK7, Myoglobin promoter (Mb), Desmin promoter, muscle creatine kinase promoter (MCK) and variants thereof, and SPc5-12 synthetic promoter.

Suitable neuronal tissue/cell specific promoters include, but are not limited to, GFAP promoter (astrocytes), SYN1 promoter (neurons), and NSE/RU5′ (mature neurons).

Other suitable CNS specific promoters can include, but are not limited to, neuroactive peptide cholecystokinin (CCK) (see e.g., Chhatawl et al. Gene Therapy volume 14, pages 575-583(2007)), a brain specific DNA MiniPromoter (such as any of those identified for brain or pan-neronal expression as in de Leeuw et al. Mol. Therapy. 1(5): 2014. doi:10.1038/mtm.2013.5), myelin basic promoter (MBP) (see e.g., von Jonquieres, G., Mersmann, N., Klugmann, C. B., Harasta, A. E., Lutz, B., Teahan, O., et al. (2013). Glial promoter selectivity following AAV-delivery to the immature brain. PLoS One 8 (6), e65646. doi: 10.1371/journal.pone.0065646), glial fibrillary acid protein (GFAP) for expression in astrocytes (see e.g., Smith-Arica, J. R., Morelli, A. E., Larregina, A. T., Smith, J., Lowenstein, P. R., Castro, M. G. (2000). Cell-type-specific and regulatable transgenesis in the adult brain: adenovirus-encoded combined transcriptional targeting and inducible transgene expression. Mol. Ther. 2 (6), 579-587. doi: 10.1006/mthe.2000.0215 and Lee, Y., Messing, A., Su, M., Brenner, M. (2008). GFAP promoter elements required for region-specific and astrocyte-specific expression. Glia 56 (5), 481-493. doi: 10.1002/glia.20622), human myelin associated glycoprotein promoter (full-length or truncated) (see e.g., von Jonquieres, G., Frohlich, D., Klugmann, C. B., Wen, X., Harasta, A. E., Ramkumar, R., et al. (2016). Recombinant human myelin-associated glycoprotein promoter drives selective AAV-mediated transgene expression in oligodendrocytes. Front. Mol. Neurosci. 9, 13. doi: 10.3389/fnmol.2016.00013), F4/80 promoter (see e.g., Rosario, A. M., Cruz, P. E., Ceballos-Diaz, C., Strickland, M. R., Siemienski, Z., Pardo, M., et al. (2016). Microglia-specific targeting by novel capsid-modified AAV6 vectors. Mol. Ther. Methods Clin. Dev. 3, 16026. doi: 10.1038/mtm.2016.26), phosphate-activated glutaminase (PAG) or the vesicular glutamate transporter (vGLUT) promoter (for about 90% glutamatergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), glutamic acid decarboxylase (GAD) promoter (for about 90% GABAergic neuron-specific expression) (see e.g., Rasmussen, M., Kong, L., Zhang, G. R., Liu, M., Wang, X., Szabo, G., et al. (2007). Glutamatergic or GABAergic neuron-specific, long-term expression in neocortical neurons from helper virus-free HSV-1 vectors containing the phosphate-activated glutaminase, vesicular glutamate transporter-1, or glutamic acid decarboxylase promoter. Brain Res. 1144, 19-32. doi: 10.1016/j.brainres.2007.01.125), MeCP2 promoter (see e.g., Gray et al. Hum Gene Ther. 2011 September; 22(9):1143-53. doi: 10.1089/hum.2010.245), and retinoblastoma gene promoter (see e.g., Jiang et al., J. Biol. Chem. 2001. 276, 593-600).

Other tissue and/or cell specific promoters are discussed elsewhere herein and can be generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the engineered targeting moiety, polypeptide, viral (e.g., AAV) capsid system described herein are typically placed under control of a plant promoter, i.e., a promoter operable in plant cells. The use of different types of promoters is envisaged. In some embodiments, inclusion of an engineered targeting moiety, polypeptide, viral (e.g., AAV) capsid system vector in a plant can be for AAV vector production purposes.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered targeting moiety, polypeptide, viral (e.g., AAV) capsid system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the engineered targeting moiety, polypeptide, viral (e.g., AAV) capsid system are found in Kawamata et al., (1997) Plant Cell Physiol 38:792-803; Yamamoto et al., (1997) Plant J 12:255-65; Hire et al., (1992) Plant Mol Biol 20:207-18; Kuster et al., (1995) Plant Mol Biol 29:759-72; and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more elements of the engineered targeting moiety, polypeptide, viral (e.g., AAV) capsid system described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing an engineered targeting moiety, polypeptide, viral (e.g., AAV) capsid polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the engineered targeting moieties, polypeptide, viral (e.g., AAV) capsid polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered targeting moiety, polypeptide, viral (e.g., AAV) capsid system polynucleotide such that the selectable marker polypeptide, when translated, is inserted between two amino acids between the N- and C-terminus of the engineered targeting moiety, polypeptide, viral (e.g., AAV) capsid polypeptide or at the N- and/or C-terminus of the engineered targeting moiety, polypeptide, viral (e.g., AAV) capsid polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered targeting moiety, polypeptide, viral (e.g., AAV) capsid system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system described herein via suitable linker, such as a glycine or glycine serine linkers as short as GS or GG up to (GGGGG)₃ (SEQ ID NO: 1709) or (GGGGS)₃ (SEQ ID NO: 1710). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered targeting moiety, polypeptide, viral (e.g., AAV) capsid polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered targeting moiety, polypeptide, viral (e.g., AAV) capsid polynucleotide(s) to specific cells, tissues, organs, etc.

### Cell-Free Vector and Polynucleotide Expression

In some embodiments, the polynucleotide encoding one or more features of the engineered targeting moiety, polypeptide, viral (e.g., AAV) capsid system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E. coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more embodiments of the engineered targeting moiety, polypeptide, viral (e.g., AAV) capsid system described herein can be codon optimized. In some embodiments, one or more polynucleotides contained in a vector (“vector polynucleotides”) described herein that are in addition to an optionally codon optimized polynucleotide encoding embodiments of the engineered targeting moiety, polypeptide, viral (e.g., AAV) capsid system described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.oijp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1):1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, muscle cells (e.g., skeletal and/or muscle cells), CNS epithelial cells (including but not limited to the cells lining the brain ventricles), nerve cells (nerves, brain cells, spinal column cells, nerve support cells (e.g., astrocytes, glial cells, Schwann cells etc.), connective tissue cells of the CNS (fat and other soft tissue padding cells of the CNS such as the meninges), stem cells and other progenitor cells, CNS immune cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, CNS tissue and/or muscle tissue and/or cells thereof. In some embodiments, the tissue types include muscle tissue and/or cells thereof. In some embodiments, the tissue types include cardiac muscle tissue and/or cells thereof. In some embodiments, the tissue types include skeletal muscle tissue and/or cells thereof. Such codon optimized sequences are within the ambit of the ordinary skilled artisan in view of the description herein. In some embodiments, the polynucleotide is codon optimized for a specific organ. Such organs include, but are not limited to, the brain. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered targeting moiety, polypeptide, viral (e.g., AAV) capsid polynucleotide of the present invention and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as an engineered targeting moiety, polypeptide, viral (e.g., AAV) capsid polynucleotide of the present invention.

### Naked Polynucleotides

In some embodiments one or more engineered targeting moiety, polypeptide, viral (e.g., AAV) capsid polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered targeting moiety, polypeptide, viral (e.g., AAV) capsid polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered targeting moiety, polypeptide, viral (e.g., AAV) capsid polynucleotide(s) of the present invention. In some embodiments, the naked polynucleotide can contain other nucleic acids and/or polynucleotides in addition to the engineered targeting moiety, polypeptide, viral (e.g., AAV) capsid polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the engineered targeting moiety, polypeptide, viral (e.g., AAV) capsid polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more engineered AAV capsid polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered targeting moiety, polypeptide, viral (e.g., AAV) capsid polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered targeting moiety, polypeptide, viral (e.g., AAV) capsid polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered targeting moieties, polypeptide, viral (e.g., AAV) capsid polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, but are not limited to, Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof.

### Chemical Carriers

In some embodiments the engineered targeting moiety, polypeptide, viral (e.g., AAV) capsid polynucleotide(s) can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered targeting moiety, polypeptide, viral (e.g. AAV) capsid polynucleotide(s) of the present invention), (2) those capable of targeting specific cells, (3) those capable of increasing delivery of the polynucleotide (such as the engineered targeting moiety, polypeptide, viral (e.g. AAV) capsid polynucleotide(s) of the present invention) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the engineered targeting moiety, polypeptide, viral (e.g., AAV) capsid system components described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticuloendothelial system. In some embodiments, the inorganic particles can be optimized to protect an entrapped molecule from degradation. The Suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an engineered targeting moiety, polypeptide, viral (e.g., AAV) capsid polynucleotide of the present invention). In some embodiments, chemical non-viral carrier systems can include a polynucleotide such as the engineered targeting moiety, polypeptide, viral (e.g., AAV) capsid polynucleotide(s) of the present invention) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immisicible liquid in another stabilized emulsifying agent and can have particles of about 200 nm that are composed of the lipid, water, and surfactant that can contain the polynucleotide to be delivered (e.g., the engineered targeting moiety, polypeptide, viral (e.g., AAV) capsid polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered targeting moiety, polypeptide, viral (e.g., AAV) capsid polynucleotides of the present invention), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the engineered AAV capsid polynucleotides describe herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the engineered targeting moiety, polypeptide, viral (e.g., AAV) capsid polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered targeting moiety, polypeptide, viral (e.g., AAV) capsid polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the engineered targeting moiety, polypeptide, viral (e.g., AAV) capsid system described herein. The viral vector can be part of a viral vector system involving multiple vectors. In some embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In some embodiments, the viral vectors are configured to produce replication incompetent viral particles for improved safety of these systems.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2, 5, or 9. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261. The engineered AAV capsids can be included in an adenoviral vector to produce adenoviral particles containing said engineered AAV capsids.

In some embodiments the vector can be a helper-dependent adenoviral vector or system thereof. These are also referred to in the field as “gutless” or “gutted” vectors and are a modified generation of adenoviral vectors (see e.g., Thrasher et al. 2006. Nature. 443:E5-7). In embodiments of the helper-dependent adenoviral vector system one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In some embodiments, the polynucleotide to be delivered via the viral particle produced from a helper-dependent adenoviral vector or system thereof can be up to about 38 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the engineered AAV capsid system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered targeting moiety, polypeptide, viral (e.g., AAV) capsid system of the present invention.

### Adeno Associated Vectors

In an embodiment, the engineered vector or system thereof can be an adeno-associated vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the promoter can be a tissue specific promoter as previously discussed. In some embodiments, the tissue specific promoter can drive expression of an engineered capsid AAV capsid polynucleotide described herein.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue- and/or organ-specific tropism.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the bias for e.g., muscle (or other non-CNS tissue or cell) can be reduced or eliminated and/or the CNS tissue or cell specificity increased such that the muscle (or other non-CNS tissue or cell) specificity appears reduced in comparison, thus enhancing the specificity for the CNS tissue or cell as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82: 5887-5911 (2008) reproduced below as Table 1. Further tropism details can be found in Srivastava. 2017. Curr. Opin. Virol. 21:75-80 as previously discussed.

TABLE 1

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In some embodiments, the AAV vector or system thereof is AAV rh.74 or AAV rh.10.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of an engineered AAV capsid system described herein are as used in the foregoing documents, such as WO 2014/093622 (PCT/US2013/074667) and are discussed in greater detail herein.

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered AAV capsid polynucleotide(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

The engineered AAV vectors and systems thereof described herein can be produced by any of these methods.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

One or more engineered AAV capsid polynucleotides can be delivered using adeno associated virus (AAV), adenovirus or other plasmid or viral vector types as previously described, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of engineered AAV capsid system components (e.g., polynucleotides encoding engineered AAV capsid and/or capsid proteins) to cells via particles. The term “particle” as used herein, refers to any suitable sized particles for delivery of the engineered AAV capsid system components described herein. Suitable sizes include macro-, micro-, and nano-sized particles. In some embodiments, any of the of the engineered AAV capsid system components (e.g., polypeptides, polynucleotides, vectors, and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other engineered capsid system molecules and formulations described elsewhere herein.

### Engineered Virus Particles Including an Engineered Viral Capsid

Also described herein are engineered virus particles (also referred to here and elsewhere herein as “engineered viral particles”) that can contain an engineered viral (e.g., AAV) capsid as described in detail elsewhere herein. Viral particles with an engineered AAV capsid are referred to herein as engineered AAV particles. It will be appreciated that the engineered viral (e.g., AAV) particles can be adenovirus-based particles, helper adenovirus-based particles, AAV-based particles, or hybrid adenovirus-based particles that contain at least one engineered AAV capsid proteins as previously described. An engineered AAV capsid is one that that contains one or more engineered AAV capsid proteins as are described elsewhere herein. In some embodiments, the engineered AAV particles can include 1-60 engineered AAV capsid proteins described herein. In some embodiments, the engineered AAV particles can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV particles can contain 0-59 wild-type AAV capsid proteins. In some embodiments, the engineered AAV particles can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins. The engineered AAV particles can thus include one or more n-mer inserts as is previously described.

The engineered AAV particle can include one or more cargo polynucleotides. Cargo polynucleotides are discussed in greater detail elsewhere herein. Methods of making the engineered AAV particles from viral and non-viral vectors are described elsewhere herein. Formulations containing the engineered virus particles are described elsewhere herein.

The engineered viral (e.g., AAV) capsid polynucleotides, other viral (e.g., AAV) polynucleotide(s), and/or vector polynucleotides can contain one or more cargo polynucleotides. The cargo polynucleotides can encode one or more polypeptides. Exemplary cargos are described in greater detail elsewhere herein. It will be appreciated that when a cargo polypeptide is described that its encoding polynucleotide can be a cargo polynucleotide described in this context. In some embodiments, the one or more cargo polynucleotides can be operably linked to the engineered viral (e.g., AAV) capsid polynucleotide(s) and can be part of the engineered viral (e.g., AAV) genome of the viral (e.g., AAV) system of the present invention. The cargo polynucleotides can be packaged into an engineered viral (e.g., AAV) particle, which can be delivered to, e.g., a cell. In some embodiments, the cargo polynucleotide can be capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g., insertional and deletional mutagenesis) techniques.

### Engineered Cells and Organisms Expressing Said Engineered Viral Capsids

Described herein are engineered cells that can include one or more of the engineered targeting moieties, polypeptides, viral (e.g., AAV) capsid polynucleotides, polypeptides, vectors, and/or vector systems described in greater detail elsewhere herein. In some embodiments, one or more of the engineered viral (e.g., AAV) capsid polynucleotides can be expressed in the engineered cells. In some embodiments, the engineered cells can be capable of producing engineered viral (e.g., AAV) capsid proteins and/or engineered viral (e.g., AAV) capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered viral (e.g., AAV) capsid polynucleotide as described elsewhere herein.

A wide variety of animals, plants, algae, fungi, yeast, etc. and animal, plant, algae, fungus, yeast cell or tissue systems may be engineered to express one or more nucleic acid constructs of the engineered targeting moiety, polypeptide, vector, viral (e.g., AAV) capsid system described herein using various transformation methods mentioned elsewhere herein. This can produce organisms that can produce engineered targeting moiety, polypeptide, vector, viral (e.g., AAV) capsid particles, such as for production purposes, engineered targeting moiety, polypeptide, vector, viral (e.g., AAV) capsid design and/or generation, and/or model organisms. In some embodiments, the polynucleotide(s) encoding one or more components of the engineered targeting moiety, polypeptide, vector, viral (e.g., AAV) capsid system described herein can be stably or transiently incorporated into one or more cells of a plant, animal, algae, fungus, and/or yeast or tissue system. In some embodiments, one or more of engineered targeting moiety, polypeptide, vector, viral (e.g., AAV) capsid system polynucleotides are genomically incorporated into one or more cells of a plant, animal, algae, fungus, and/or yeast or tissue system. Further embodiments of the modified organisms and systems are described elsewhere herein. In some embodiments, one or more components of the engineered targeting moiety, polypeptide, vector, viral (e.g., AAV) capsid system described herein are expressed in one or more cells of the plant, animal, algae, fungus, yeast, or tissue systems.

### Engineered Cells

Described herein are various embodiments of engineered cells that can include one or more of the engineered targeting moieties, polypeptide, vector, viral (e.g., AAV) capsid system polynucleotides, polypeptides, vectors, and/or vector systems described elsewhere herein. In some embodiments, the cells can express one or more of the engineered targeting moieties, polypeptide, vector, viral (e.g., AAV) capsid polynucleotides and can produce one or more engineered targeting moiety, polypeptide, vector, viral (e.g., AAV) capsid particles, which are described in greater detail herein. Such cells are also referred to herein as “producer cells”. It will be appreciated that these engineered cells are different from “modified cells” described elsewhere herein in that the modified cells are not necessarily producer cells (i.e. they do not make engineered viral (e.g., AAV) particles) unless they include one or more of the engineered targeting moiety, polypeptide, viral (e.g., AAV) capsid polynucleotides, engineered targeting moiety, polypeptide, viral (e.g., AAV) capsid vectors or other vectors described herein that render the cells capable of producing an engineered viral (e.g., AAV) capsid particle or other particles described herein. Modified cells can be recipient cells of an engineered viral (e.g., AAV) capsid particles and can, in some embodiments, be modified by the engineered viral (e.g., AAV) capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered targeting moiety, polypeptide, viral (e.g., AAV) capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In some embodiments, the organism is a host of a virus (e.g., an AAV).

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Pane1, PC-3, TF1, CTLL-2, CIR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, the engineered cell is a muscle cell (e.g., cardiac muscle, skeletal muscle, and/or smooth muscle), bone cell, blood cell, immune cell (including but not limited to B cells, macrophages, T-cells, CAR-T cells, and the like), kidney cells, bladder cells, lung cells, heart cells, liver cells, brain cells, neurons, skin cells, stomach cells, neuronal support cells, intestinal cells, epithelial cells, endothelial cells, stem or other progenitor cells, adrenal gland cells, cartilage cells, and combinations thereof.

In some embodiments, the engineered cell can be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when an engineered targeting moiety, polypeptide, vector, viral (e.g., AAV) capsid particle is produced it can package one or more cargo polynucleotides that can be related to the desired physiological and/or biological characteristic and/or capable of modifying the desired physiological and/or biological characteristic. Thus, the cargo polynucleotides of the produced engineered viral (e.g., AAV) or other particle can be capable of transferring the desired characteristic to a recipient cell. In some embodiments, the cargo polynucleotides are capable of modifying a polynucleotide of the engineered cell such that the engineered cell has a desired physiological and/or biological characteristic.

In some embodiments, a cell transfected with one or more vectors described herein is used to establish a new cell line comprising one or more vector-derived sequences.

The engineered cells can be used to produce engineered targeting moieties, polypeptides, viral (e.g., AAV) capsid polynucleotides, vectors, and/or particles. In some embodiments, the engineered targeting moieties, polypeptides, viral (e.g., AAV) capsid polynucleotides, vectors, and/or particles are produced, harvested, and/or delivered to a subject in need thereof. In some embodiments, the engineered cells are delivered to a subject. Other uses for the engineered cells are described elsewhere herein. In some embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

Component(s) of the engineered targeting moieties, polypeptides, viral (e.g., AAV) capsid system, engineered cells, engineered viral (e.g., AAV) particles, and/or combinations thereof can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, uL, mL, or L.

In embodiments, were engineered AAV capsid particles are included in the formulation, the formulation can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered viral (e.g. AAV) capsids, viral (e.g. AAV) or otherr particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

In embodiments where there is an auxiliary active agent contained in the pharmaceutical formulation in addition to the one or more of the polypeptides, polynucleotides, compositions, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, manitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subginigival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### Kits

Also described herein are kits that contain one or more of the one or more of the polypeptides, polynucleotides, vectors, cells, or other components described herein and combinations thereof and pharmaceutical formulations described herein. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In some embodiments, the kit comprises a vector system and instructions for using the kit. In some embodiments, the vector system includes a regulatory element operably linked to one or more engineered targeting moiety, polypeptide, viral (e.g., AAV) delivery system polynucleotides, as described elsewhere herein and, optionally, a cargo molecule, which can optionally be operably linked to a regulatory element. The one or more engineered targeting moiety, polypeptide, viral (e.g., AAV) delivery system polynucleotides, can be included on the same or different vectors as a cargo molecule capable of being delivered by the engineered targeting moiety, polypeptide, viral (e.g., AAV) delivery system described herein in embodiments containing a cargo molecule within the kit.

In some embodiments, the kit comprises a vector system and instructions for using the kit. In some embodiments, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a Cas9 CRISPR complex to a target sequence in a eukaryotic cell, wherein the Cas9 CRISPR complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said CRISPR enzyme in a detectable amount in the nucleus of a eukaryotic cell. In some embodiments, the CRISPR enzyme is a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild-type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

### Methods of Use

### General Discussion

The compositions containing the CNS-muscle or muscle specific targeting moieties described herein (e.g., the engineered targeting moiety system polynucleotides, polypeptides, vector(s), engineered cells, engineered viral (e.g., AAV) capsids, and viral and other particles) can be used generally to package and/or deliver one or more cargo polynucleotides to a recipient cell. In some embodiments, delivery, is done in a cell-specific manner based upon the specificity of the targeting moiety(ies) included. In some embodiments, the cell-specificity is conferred via the n-mer insert(s) included in the targeting moiety as previously discussed. In some embodiments, delivery is done in cell-specific manner based upon the tropism of the engineered viral (e.g., AAV) capsid. In some embodiments, engineered targeting moiety(ies), polypeptides, viral (e.g., AAV) capsids, particles, viral (e.g., AAV) particles, compositions thereof, and/or cells discussed herein can be administered to a subject or a cell, tissue, and/or organ and facilitate the transfer and/or integration of the cargo polynucleotide to the recipient cell. In other embodiments, engineered cells capable of producing engineered targeting moiety(ies), polypeptides, viral (e.g., AAV) capsids, particles, viral (e.g., AAV) particles and/or compositions thereof can be generated from engineered targeting moiety system molecules (e.g., polynucleotides, vectors, and vector systems, etc.). In some embodiments, the engineered targeting moiety(ies), polypeptides, viral (e.g., AAV) capsids, particles, viral (e.g., AAV) particles and/or compositions thereof can be delivered to a subject or a cell, tissue, and/or organ. When delivered to a subject, they engineered delivery system molecule(s) can transform a subject's cell in vivo or ex vivo to produce an engineered cell that can be capable of making an engineered targeting moiety(ies), polypeptides, viral (e.g., AAV) capsids, particles, viral (e.g., AAV) particles and/or compositions thereof, which can be released from the engineered cell and deliver cargo molecule(s) to a recipient cell in vivo or produce personalized engineered polypeptides, viral (e.g., AAV) particles, and/or other particles for reintroduction into the subject from which the recipient cell was obtained. In some embodiments, an engineered cell can be delivered to a subject, where it can release produced engineered targeting moieties, polypeptides, viral (e.g., AAV) particles, and/or other particles such that they can then deliver a cargo (e.g., cargo polynucleotide(s)) to a recipient cell. These general processes can be used in a variety of ways to treat and/or prevent disease or a symptom thereof in a subject, generate model cells, generate modified organisms, provide cell selection and screening assays, in bioproduction, and in other various applications.

In some embodiments, the engineered targeting moieties, polypeptides, viral (e.g., AAV) particles, and/or other particles, polynucleotides, vectors, and systems thereof can be used to generate engineered AAV capsid variant libraries that can be mined for variants with a desired cell-specificity, such as both CNS and muscle specificity or muscle cell (e.g., cardiac and/or skeletal muscle cell) specificity. The description provided herein as supported by the various Examples can demonstrate that one having a desired cell-specificity in mind could utilize the present invention as described herein to obtain a capsid with the desired cell-specificity, such as both CNS and muscle specificity or muscle cell (e.g., cardiac and/or skeletal muscle cell) specificity.

### Therapeutics

In some embodiments, one or more molecules of the engineered delivery system, engineered targeting moieties, polypeptides, viral (e.g., AAV) particles, and/or other particles, polynucleotides, vectors, systems thereof, engineered cells, and/or formulations thereof described herein can be delivered to a subject in need thereof as a therapy for one or more diseases. In some embodiments, the disease to be treated is a genetic or epigenetic based disease. In some embodiments, the disease to be treated is not a genetic or epigenetic based disease. In some embodiments, one or more molecules of the engineered delivery system, engineered targeting moieties, polypeptides, viral (e.g., AAV) particles, and/or other particles, polynucleotides, vectors, and systems thereof, engineered cells, and/or formulations thereof described herein can be delivered to a subject in need thereof as a treatment or prevention (or as a part of a treatment or prevention) of a disease. It will be appreciated that the specific disease to be treated and/or prevented by delivery of an engineered cell and/or engineered can be dependent on the cargo molecule packaged into an engineered AAV capsid particle.

Generally, the compositions described herein can be used in a therapy for treating a CNS and/or a muscle (such as a cardiac muscle or skeletal muscle) disease, disorder, or a symptom thereof. It will be appreciated that a CNS disease or disorder refers to any disease or disorder whose pathology involves or affects one or more cell types of the central nervous system. In some embodiments, the CNS disease or disorder is one whose primary pathology involves one or more cell types of the CNS. In some embodiments, one or more other cell types outside of the CNS are involved in the pathology of the CNS diseases, such as a muscle cell or peripheral nervous system cell. In some embodiments, the CNS disease or disorder can be caused by one or more genetic abnormalities. In some embodiments, the CNS disease or disorder is not caused by a genetic abnormality. Non-genetic cause of diseases includes infection, cancer, physical trauma and others that will be appreciated by those of skill in the art. It also will be appreciated that gene modification approaches to treating disease can be applied to treat and/or prevent both genetic diseases and non-genetic diseases. For example, in the case of non-genetic diseases, a gene therapy approach can be used to modify the cause of the non-genetic disease (e.g., a cancer or infectious organism) such that the cause is no longer disease causing (e.g., by eliminating or rendering non-functional the cancer cells or infectious organism).

Exemplary CNS diseases and disorders include, without limitation, Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, or a combination thereof. Others are described elsewhere herein and/or will be appreciated by those of ordinary skill in the art in view of the description provided herein).

Genetic diseases that can be treated are discussed in greater detail elsewhere herein (see e.g., discussion on Gene-modification based-therapies below). Other diseases can include, but are not limited to, any of the following: cancer (such as glioblastoma or other brain or CNS cancers), Acubetivacter infections, actinomycosis, African sleeping sickness, AIDS/HIV, ameobiasis, Anaplasmosis, Angiostrongyliasis, Anisakiasis, Anthrax, Acranobacterium haemolyticum infection, Argentine hemorrhagic fever, Ascariasis, Aspergillosis, Astrovirus infection, Babesiosis, Bacterial meningitis, Bacterial pneumonia, Bacterial vaginosis, Bacteroides infection, balantidiasis, Bartonellosis, Baylisascaris infection, BK virus infection, Black Piedra, Blastocytosis, Blastomycosis, Bolivian hemorrhagic fever, Botulism, Brazillian hemmorhagic fever, brucellosis, Bubonic plague, Burkholderia infection, buruli ulcer, calicivirus invention, campylobacteriosis, Candidasis, Capillariasis, Carrion's disease, Cat-scratch disease, cellulitis, Chagas Disease, Chancroid, Chickenpox, Chikungunya, Chlamydia, Chlamydia pneumoniae, Cholera, Chromoblastomycosis, Chytridiomycosis, Clonochiasis, Clostridium difficile colitis, Coccidioidomycosis, Colorado tick fever, rhinovirus/coronavirus invection (common cold), Cretzfeldt-Jakob disease, Crimean-congo hemorrhagic fever, Cryptococcosis, Cryptosporidosis, Cutaneous larva migrans (CLM), cyclosporiasis, cysticercosis, cytomegalovirus infection, Dengue fever, Desmodesmus infection, Dientamoebiasis, Diptheria, Diphylobothriasis, Dracunculiasis, Ebola, Echinococcosis, Ehrlichiosis, Enterobiasis, Enterococcus infection, Enterovirus infection, Epidemic typhus, Erthemia Infectisoum, Exanthem subitum, Fasciolasis, Fasciolopsiasis, fatal familial insomnia, filarisis, Clostridum perfingens infection, Fusobacterium infection, Gas gangrene (clostridial myonecrosis), Geotrichosis, Gerstmann-Straussler-Scheinker syndrome, Giardasis, Glanders, Gnathostomiasis, Gonorrhea, Granuloma inguinales, Group A streptococcal infection, Group B streptococcal infection, Haemophilus influenzae infection, Hand, foot, and mouth disease, hanta virus pulmonary syndrome, heartland virus disease, Helicobacter pylori infection, hemorrhagi fever with renal syndrome, Hendra virus infection, Hepatits (all groups A, B, C, D, E), hepes simplex, histoplasmosis, hookworm infection, human bocavirus infection, human ewingii erlichosis, Human granulocytic anaplasmosis, human metapneymovirus infection, human monocytic ehrlichosis, human papaloma virus, Hymenolepiasis, Epstein-Barr infection, mononucleosis, influenza, isoporisis, Kawasaki disease, Kingell kingae infection, Kuru, Lasas fever, Leginollosis (Legionnaires's disease and Potomac Fever), Leishmaniasis, Leprosy, Leptospirosis, Listeriosis, Lyme disease, lymphatic filariasis, lymphocytic choriomeningitis, Malaria, Marburg hemorrhagic feaver, measals, Middle East respiratory syndrome, Meliodosis, menigitis, Menigococcal disease, Metagonimiasis, Microsporidosis, Molluscum contagiosum, Monkeypox, Mumps, Murine typhus, Mycoplasma pneumonia, Mycoplasma genitalium infection, Mycetoma, Myiasis, Conjunctivitis, Nipah virus infection, Norovirus, Variant Creutzfeldt-Jakob disease, Nocardosis, Onchocerciasis, Opisthorchiasis, Paracoccidioidomycosis, Paragonimiasis, Pasteurellosis, Pdiculosisi capitis, Pediculosis corpis, Pediculosis pubis, pelvic inflammatory disease, pertussis, plague, pneumococcal infection, pneumocystis pneumonia, pneumonia, poliomyelitis, prevotella infection, primary amoebic menigoencephalitis, progressive multifocal leukoencephalopathy, Psittacosis, Qfever, rabies, relapsing fever, respiratory syncytial virus infection, rhinovirus infection, rickettsial infection, Rickettsialpox, Rift Valley Fever, Rocky Mountain Spotted Fever, Rotavirus infection, Rubella, Salmonellosis, SARS, Scabies, Scarlet fever, Schistosomiais, Sepsis, Shigellosis, Shingles, Smallpox, Sporotrichosisi, Staphlococcol infection (including MRSA), strongyloidiasis, subacute sclerosing panecephalitis, Syphillis, Taeniasis, tetanus, Trichophyton species infection, Tocariasis, Toxoplasmosis, Trachoma, Trichinosis, Trichuriasis, Tuberculosis, Tularemia, Typhoid Fever, Typhus Fever, Ureaplasma urealyticum infection, Valley fever, Venezuelan equine encephalitis, Venezuelan hemorrhagic fever, Vibrio species infection, Viral pneumonia, West Nile Fever, White Piedra, Yersinia pseudotuberculosis, Yersiniosis, Yellow fever, Zeaspora, Zika fever, Zygomycosis and combninations thereof.

Other diseases and disorders that can be treated using embodiments of the present invention include, but are not limited to, endocrine diseases (e.g., Type I and Type II diabetes, gestational diabetes, hypoglycemia. Glucagonoma, Goitre, Hyperthyroidism, hypothyroidism, thyroiditis, thyroid cancer, thyroid hormone resistance, parathyroid gland disorders, Osteoporosis, osteitis deformans, rickets, ostomalacia, hypopituitarism, pituitary tumors, etc.), skin conditions of infections and non-infectioua origin, eye diseases of infectious or non-infectious origin, gastrointestinal disorders of infectious or non-infectious origin, cardiovascular diseases of infectious or non-infectious origin, brain and neuron diseases of infectious or non-infectious origin, nervous system diseases of infectious or non-infectious origin, muscle diseases of infectious or non-infectious origin, bone diseases of infectious or non-infectious origin, reproductive system diseases of infectious or non-infectious origin, renal system diseases of infectious or non-infectious origin, blood diseases of infectious or non-infectious origin, lymphatic system diseases of infectious or non-infectious origin, immune system diseases of infectious or non-infectious origin, mental-illness of infectious or non-infectious origin and the like.

In some embodiments, the disease to be treated is a CNS or CNS related disease or disorder, such as a genetic CNS disease or disorder. Such CNS or CNS related disease (including genetic CNS disease or disorders are described in greater detail elsewhere herein.

Other diseases and disorders will be appreciated by those of skill in the art.

### Adoptive Cell Therapies

Generally speaking, adoptive cell transfer involves the transfer of cells (autologous, allogeneic, and/or xenogeneic) to a subject. The cells may or may not be modified and/or otherwise manipulated prior to delivery to the subject. Manipulation can include genetic modification by one or more gene modifying agents. Exemplary gene modifying agents and systems are described in greater detail elsewhere herein and will be appreciated by those of ordinary skill in the art. Such gene or other modification compositions or systems can be delivered to a cell to be modified for adoptive therapy by one or more of the compositions described herein containing a CNS-muscle or muscle specific targeting moiety.

In some embodiments, an engineered cell as described herein can be included in an adoptive cell transfer therapy. In some embodiments, an engineered cell as described herein can be delivered to a subject in need thereof. In some embodiments, the cell can be isolated from a subject, manipulated in vitro such that it is capable of generating an engineered AAV capsid particle described herein to produce an engineered cell and delivered back to the subject in an autologous manner or to a different subject in an allogeneic or xenogeneic manner. The cell isolated, manipulated, and/or delivered can be a eukaryotic cell. The cell isolated, manipulated, and/or delivered can be a stem cell. The cell isolated, manipulated, and/or delivered can be a differentiated cell. The cell isolated, manipulated, and/or delivered can be a nervous system cell, such as a central nervous system cell, including but not limited to a neuron, a glial cell, an astrocyte, a Schwann cell, a microglial cell, or other neuron support cell, and/or other brain or CNS cell, a muscle cell (e.g., a skeletal muscle cell and/or a cardiac muscle cell), or both a CNS and muscle cell. Other specific cell types will instantly be appreciated by one of ordinary skill in the art.

In some embodiments, the isolated cell can be manipulated such that it becomes an engineered cell as described elsewhere herein (e.g., contain and/or express one or more engineered delivery system molecules or vectors described elsewhere herein). Methods of making such engineered cells are described in greater detail elsewhere herein.

### Gene Drives

The present invention also contemplates use of the engineered delivery system molecules, vectors, engineered cells, and/or engineered AAV capsid particles described herein to generate a gene drive via delivery of one or more cargo polynucleotides or production of engineered AAV capsid particles with one or more cargo polynucleotides capable of producing a gene drive. In some embodiments, the gene drive can be a Cas-mediated RNA-guided gene drive e.g., Cas- to provide RNA-guided gene drives, for example in systems analogous to gene drives described in PCT Patent Publication WO 2015/105928. Systems of this kind may for example provide methods for altering eukaryotic germline cells, by introducing into the germline cell a nucleic acid sequence encoding an RNA-guided DNA nuclease and one or more guide RNAs. The guide RNAs may be designed to be complementary to one or more target locations on genomic DNA of the germline cell. The nucleic acid sequence encoding the RNA guided DNA nuclease and the nucleic acid sequence encoding the guide RNAs may be provided on constructs between flanking sequences, with promoters arranged such that the germline cell may express the RNA guided DNA nuclease and the guide RNAs, together with any desired cargo-encoding sequences that are also situated between the flanking sequences. The flanking sequences will typically include a sequence which is identical to a corresponding sequence on a selected target chromosome, so that the flanking sequences work with the components encoded by the construct to facilitate insertion of the foreign nucleic acid construct sequences into genomic DNA at a target cut site by mechanisms such as homologous recombination, to render the germline cell homozygous for the foreign nucleic acid sequence.

In this way, gene-drive systems are capable of introgressing desired cargo genes throughout a breeding population (Gantz et al., 2015, Highly efficient Cas9-mediated gene drive for population modification of the malaria vector mosquito Anopheles stephensi, PNAS 2015, published ahead of print Nov. 23, 2015, doi:10.1073/pnas.1521077112; Esvelt et al., 2014, Concerning RNA-guided gene drives for the alteration of wild populations eLife 2014; 3:e03401). In select embodiments, target sequences may be selected which have few potential off-target sites in a genome. Targeting multiple sites within a target locus, using multiple guide RNAs, may increase the cutting frequency and hinder the evolution of drive resistant alleles. Truncated guide RNAs may reduce off-target cutting. Paired nickases may be used instead of a single nuclease, to further increase specificity. Gene drive constructs (such as gene drive engineered delivery system constructrs) may include cargo sequences encoding transcriptional regulators, for example to activate homologous recombination genes and/or repress non-homologous end-joining. Target sites may be chosen within an essential gene, so that non-homologous end-joining events may cause lethality rather than creating a drive-resistant allele. The gene drive constructs can be engineered to function in a range of hosts at a range of temperatures (Cho et al. 2013, Rapid and Tunable Control of Protein Stability in Caenorhabditis elegans Using a Small Molecule, PLoS ONE 8(8): e72393. doi: 10.1371/journal.pone.0072393).

### Transplantation and Xenotransplantation

The engineered AAV capsid system molecules, vectors, engineered cells, and/or engineered delivery particles described herein, can be used to deliver cargo polynucleotides and/or otherwise be involved in modifying tissues for transplantation between two different persons (transplantation) or between species (xenotransplantation). Such techniques for generation of transgenic animals are described elsewhere herein. Interspecies transplantation techniques are generally known in the art. For example, RNA-guided DNA nucleases can be delivered using via engineered AAV capsid polynucleotides, vectors, engineered cells, and/or engineered AAV capsid particles described herein and can be used to knockout, knockdown or disrupt selected genes in an organ for transplant (e.g. ex vivo (e.g. after harvest but before transplantation) or in vivo (in donor or recipient)), animal, such as a transgenic pig (such as the human heme oxygenase-1 transgenic pig line), for example by disrupting expression of genes that encode epitopes recognized by the human immune system, i.e. xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Where it is interspecies transplantation (such as human to human) the engineered AAV capsid system molecules, vectors, engineered cells, and/or engineered delivery particles described herein, can be used to deliver cargo polynucleotides and/or otherwise be involved to modify the tissue to be transplanted. In some embodiments, the modification can include modifying one or more HLA antigens or other tissue type determinants, such that the immunogenic profile is more similar or identical to the recipient's immunogenic profile than to the donor's so as to reduce the occurrence of rejection by the recipient. Relevant tissue type determinants are known in the art (such as those used to determine organ matching) and techniques to determine the immunogenic profile (which is made up of the expression signature of the tissue type determinants) are generally known in the art.

In some embodiments, the donor (such as before harvest) or recipient (after transplantation) can receive one or more of the engineered AAV capsid system molecules, vectors, engineered cells, and/or engineered delivery particles described herein that are capable of modifying the immunogenic profile of the transplanted cells, tissue, and/or organ. In some embodiments, the transplanted cells, tissue, and/or organ can be harvested from the donor and the engineered AAV capsid system molecules, vectors, engineered cells, and/or engineered delivery particles described herein capable of modifying the harvested cells, tissue, and/or organ to be, for example, less immunogenic or be modified to have some specific characteristic when transplanted in the recipient can be delivered to the harvested cells, tissue, and/or organ ex vivo. After delivery the cells, tissue, and/or organs can be transplanted into the donor.

Gene Modification and Treatment of Diseases with Genetic or Epigenetic Embodiments that Affect the CNS, Brain, and/or Neurons

The engineered delivery system molecules, vectors, engineered cells, and/or engineered delivery particles described herein (e.g., those with one or more targeting moieties, such as a CNS-specific targeting moiety described herein) can be used to modify genes or other polynucleotides and/or treat diseases of the CNS, brain, and/or neurons with genetic and/or epigenetic embodiments. As described elsewhere herein the cargo molecule can be a polynucleotide that can be delivered to a cell and, in some embodiments, be integrated into the genome of the cell. In some embodiments, the cargo molecule(s) can be one or more CRISPR-Cas system components. In some embodiments, the CRISPR-Cas components, when delivered by an engineered AAV capsid particles described herein can be optionally expressed in the recipient cell and act to modify the genome of the recipient cell in a sequence specific manner. In some embodiments, the cargo molecules that can be packaged and delivered by the engineered AAV capsid particles described herein can facilitate/mediate genome modification via a method that is not dependent on CRISPR-Cas. Such non-CRISPR-Cas genome modification systems will instantly be appreciated by those of ordinary skill in the art and are also, at least in part, described elsewhere herein. In some embodiments, modification is at a specific target sequence. In other embodiments, modification is at locations that appear to be random throughout the genome.

Examples of CNS, brain, and/or neuronal disease-associated genes and polynucleotides that can be modified using the engineered delivery AAV delivery system molecules, vectors, capsids, engineered cells, and/or engineered delivery particles described herein are described below.

In some embodiments, a therapeutic or preventive, such as the engineered AAV capsids and systems thereof as described elsewhere herein, can be delivered to a subject in need thereof or a cell thereof to treat a brain, neuron, neurological, and/or central nervous system disease or disorder (CNS). In some embodiments the brain, neuron, neurological, and/or CNS disease or disorder can be caused, directly or indirectly, by one or mutations in one or more of the following genes as compared to normal or non-pathological variant of the same: in the case of Amyotrophic lateral sclerosis (ALS): SOD1, ALS2, STEX, FUS, TARDBP, VEGF (VEGF-a, VEGF-b, VEGF-c); in the case of Alzheimer's disease: E1, CHIP, UCH, UBB, Tau, LRP, PICALM, Clusterin, PSi, SORL1, CR1, Vldlr, Ubal, Uba3, CHIP28, Aqp1, Uchl1, Uchl3, APP, AAA, CVAP, AD1, APOE, AD2, PSEN2, AD4, STM2, APBB2, FE65L1, NOS3, PLAU, URK, ACE, DCP1, ACE1, MPO, PACIPI, PAXIPIL, PTIP, A2M, BLMH, BMH, PSEN1, AD3); in the case of Autism: Mecp2, BZRAP1, MDGA2, Sema5A, Neurexin 1, GLO1, MECP2, RTT, PPMX, MRX16, MRX79, NLGN3, NLGN4, KIAA1260, AUTSX2; in the case of Fragile X Syndrome: FMR2, FXR1, FXR2, mGLUR5; in the case of Huntington's disease and disease like disorders: HD, IT15, PRNP, PRIP, JPH3, JP3, HDL2, TBP, SCA17); in the case of Parkinson's disease: NR4A2, NURR1, NOT, TINUR, SNCAIP, TBP, SCA17, SNCA, NACP, PARK1, PARK4, DJ1, PARK7, LRRK2, PARK8, PINK1, PARK6, UCHL1, PARK5, SNCA, NACP, PARK1, PARK4, PRKN, PARK2, PDJ, DBH, NDUFV2, PINK1, x-synuclein); in the case of Rett syndrome: MECP2, RTT, PPMX, MRX16, MRX79, CDKL5, STK9, MECP2, RTT, PPMX, MRX16, MRX79, x-Synuclein, DJ-1; in the case of Schizophrenia: Neuregulin1 (Nrg1), Erb4 (receptor for Neuregulin), Complexin1 (Cplxl), Tphl Tryptophan hydroxylase, Tph2, Tryptophan hydroxylase 2, Neurexin 1, GSK3, GSK3a, GSK3b, 5-HTT (Slc6a4), COMT, DRD (Drdla), SLC6A3, DAOA, DTNBP1, Dao (Dao 1)); in the case of Secretase Related Disorders (APH-1 (alpha and beta), Presenilin (Psen1), nicastrin, (Ncstn), PEN-2, Nos1, Parp1, Nat1, Nat2); in the case of Trinucleotide Repeat Disorders (HTT (Huntington's Dx), SBMA/SMAX1/AR (Kennedy's Dx), FXN/X25 (Friedrich's Ataxia), ATX3 (Machado-Joseph's Dx), ATXN1 and ATXN2 (spinocerebellar ataxias), DMPK (myotonic dystrophy), Atrophin-1 and Atn1 (DRPLA Dx), CBP (Creb-BP—global instability), VLDLR (Alzheimer's), Atxn7, Atxn10); in the case of diseases or disorders associated with or involving aberrant or abnormal axonal guidance signaling in the brain, neurons, and/or CNS: PRKCE; ITGAM; ROCK1; ITGA5; CXCR4; ADAM12; IGF1; RAC1; RAPlA; EIF4E; PRKCZ; NRP1; NTRK2; ARHGEF7; SMO; ROCK2; MAPK1; PGF; RAC2; PTPN11; GNAS; AKT2; PIK3CA; ERBB2; PRKCI; PTK2; CFL1; GNAQ; PIK3CB; CXCL12; PIK3C3; WNT11; PRKD1; GNB2L1; ABL1; MAPK3; ITGA1; KRAS; RHOA; PRKCD; PIK3C2A; ITGB7; GLI2; PXN; VASP; RAF1; FYN; ITGB1; MAP2K2; PAK4; ADAM17; AKT1; PIK3R1; GLI1; WNT5A; ADAM10; MAP2K1; PAK3; ITGB3; CDC42; VEGFA; ITGA2; EPHA8; CRKL; RND1; GSK3B; AKT3; PRKCA; in the case of diseases or disorders associated with or involving aberrant or abnormal actin cytoskeleton signaling in the brain, neurons, and/or CNS: ACTN4; PRKCE; ITGAM; ROCK1; ITGA5; IRAK1; PRKAA2; EIF2AK2; RAC1; INS; ARHGEF7; GRK6; ROCK2; MAPK1; RAC2; PLK1; AKT2; PIK3CA; CDK8; PTK2; CFL1; PIK3CB; MYH9; DIAPH1; PIK3C3; MAPK8; F2R; MAPK3; SLC9A1; ITGA1; KRAS; RHOA; PRKCD; PRKAA1; MAPK9; CDK2; PIM1; PIK3C2A; ITGB7; PPP1CC; PXN; VIL2; RAF1; GSN; DYRKIA; ITGB1; MAP2K2; PAK4; PIP5K1A; PIK3R1; MAP2K1; PAK3; ITGB3; CDC42; APC; ITGA2; TTK; CSNK1A1; CRKL; BRAF; VAV3; SGK; in the case of diseases or disorders associated with or involving Huntington's Disease signaling: PRKCE; IGF1; EP300; RCOR1; PRKCZ; HDAC4; TGM2; MAPK1; CAPNS1; AKT2; EGFR; NCOR2; SP1; CAPN2; PIK3CA; HDAC5; CREB1; PRKCI; HSPA5; REST; GNAQ; PIK3CB; PIK3C3; MAPK8; IGF1R; PRKD1; GNB2L1; BCL2L1; CAPN1; MAPK3; CASP8; HDAC2; HDAC7A; PRKCD; HDAC11; MAPK9; HDAC9; PIK3C2A; HDAC3; TP53; CASP9; CREBBP; AKT1; PIK3R1; PDPK1; CASP1; APAF1; FRAP1; CASP2; JUN; BAX; ATF4; AKT3; PRKCA; CLTC; SGK; HDAC6; CASP3; in the case of diseases or disorders associated with or involving aberrant, pathologic, and/or abnormal apoptosis regulation and/or signaling in the brain, neurons, and/or CNS and/or diseases or disorders thereof: PRKCE; ROCK1; BID; IRAK1; PRKAA2; EIF2AK2; BAK1; BIRC4; GRK6; MAPK1; CAPNS1; PLK1; AKT2; IKBKB; CAPN2; CDK8; FAS; NFKB2; BCL2; MAP3K14; MAPK8; BCL2L1; CAPN1; MAPK3; CASP8; KRAS; RELA; PRKCD; PRKAA1; MAPK9; CDK2; PIM1; TP53; TNF; RAF1; IKBKG; RELB; CASP9; DYRKIA; MAP2K2; CHUK; APAF1; MAP2K1; NFKB1; PAK3; LMNA; CASP2; BIRC2; TTK; CSNK1A1; BRAF; BAX; PRKCA; SGK; CASP3; BIRC3; PARP1; in the case of diseases or disorders associated with or involving aberrant, pathologic, and/or abnormal leukocyte extravasation signaling in the brain, neurons, and/or CNS and/or diseases or disorders thereof: ACTN4; CD44; PRKCE; ITGAM; ROCK1; CXCR4; CYBA; RAC1; RAPlA; PRKCZ; ROCK2; RAC2; PTPN11; MMP14; PIK3CA; PRKCI; PTK2; PIK3CB; CXCL12; PIK3C3; MAPK8; PRKD1; ABL1; MAPK10; CYBB; MAPK13; RHOA; PRKCD; MAPK9; SRC; PIK3C2A; BTK; MAPK14; NOX1; PXN; VIL2; VASP; ITGB1; MAP2K2; CTNND1; PIK3R1; CTNNB1; CLDN1; CDC42; F11R; ITK; CRKL; VAV3; CTTN; PRKCA; MMP1; MMP9; in the case of diseases or disorders associated with or involving aberrant, pathologic, and/or abnormal integrin signaling in the brain, neurons, and/or CNS and/or diseases or disorders thereof: ACTN4; ITGAM; ROCK1; ITGA5; RAC1; PTEN; RAPlA; TLN1; ARHGEF7; MAPK1; RAC2; CAPNS1; AKT2; CAPN2; PIK3CA; PTK2; PIK3CB; PIK3C3; MAPK8; CAV1; CAPN1; ABL1; MAPK3; ITGA1; KRAS; RHOA; SRC; PIK3C2A; ITGB7; PPP1CC; ILK; PXN; VASP; RAF1; FYN; ITGB1; MAP2K2; PAK4; AKT1; PIK3R1; TNK2; MAP2K1; PAK3; ITGB3; CDC42; RND3; ITGA2; CRKL; BRAF; GSK3B; AKT3; in the case of diseases or disorders associated with or involving aberrant, pathologic, and/or abnormal acute phase response signaling in the brain, neurons, and/or CNS and/or diseases or disorders thereof: IRAK1; SOD2; MYD88; TRAF6; ELK1; MAPK1; PTPN11; AKT2; IKBKB; PIK3CA; FOS; NFKB2; MAP3K14; PIK3CB; MAPK8; RIPK1; MAPK3; IL6ST; KRAS; MAPK13; IL6R; RELA; SOCS1; MAPK9; FTL; NR3C1; TRAF2; SERPINE1; MAPK14; TNF; RAF1; PDK1; IKBKG; RELB; MAP3K7; MAP2K2; AKT1; JAK2; PIK3R1; CHUK; STAT3; MAP2K1; NFKB1; FRAP1; CEBPB; JUN; AKT3; IL1R1; IL6; in the case of diseases or disorders associated with or involving aberrant, pathologic, and/or abnormal PTEN signaling in the brain, neurons, and/or CNS and/or diseases or disorders thereof: ITGAM; ITGA5; RAC1; PTEN; PRKCZ; BCL2L11; MAPK1; RAC2; AKT2; EGFR; IKBKB; CBL; PIK3CA; CDKN1B; PTK2; NFKB2; BCL2; PIK3CB; BCL2L1; MAPK3; ITGA1; KRAS; ITGB7; ILK; PDGFRB; INSR; RAF1; IKBKG; CASP9; CDKN1A; ITGB1; MAP2K2; AKT1; PIK3R1; CHUK; PDGFRA; PDPK1; MAP2K1; NFKB1; ITGB3; CDC42; CCND1; GSK3A; ITGA2; GSK3B; AKT3; FOXOl; CASP3; RPS6KB1; in the case of diseases or disorders associated with or involving aberrant, pathologic, and/or abnormal p53 signaling in the brain, neurons, and/or CNS and/or diseases or disorders thereof: PTEN; EP300; BBC3; PCAF; FASN; BRCA1; GADD45A; BIRC5; AKT2; PIK3CA; CHEK1; TP53INP1; BCL2; PIK3CB; PIK3C3; MAPK8; THBS1; ATR; BCL2L1; E2F1; PMAIP1; CHEK2; TNFRSF1OB; TP73; RBl; HDAC9; CDK2; PIK3C2A; MAPK14; TP53; LRDD; CDKN1A; HIPK2; AKT1; PIK3R1; RRM2B; APAF1; CTNNB1; SIRT1; CCND1; PRKDC; ATM; SFN; CDKN2A; JUN; SNAI2; GSK3B; BAX; AKT3; in the case of diseases or disorders associated with or involving aberrant, pathologic, and/or abnormal aryl hydrocarbon receptor signaling in the brain, neurons, and/or CNS and/or diseases or disorders thereof: HSPB1; EP300; FASN; TGM2; RXRA; MAPK1; NQO1; NCOR2; SP1; ARNT; CDKN1B; FOS; CHEK1; SMARCA4; NFKB2; MAPK8; ALDH1A1; ATR; E2F1; MAPK3; NRIP1; CHEK2; RELA; TP73; GSTP1; RB1; SRC; CDK2; AHR; NFE2L2; NCOA3; TP53; TNF; CDKN1A; NCOA2; APAF1; NFKB1; CCND1; ATM; ESR1; CDKN2A; MYC; JUN; ESR2; BAX; IL6; CYP1B1; HSP90AA1; in the case of diseases or disorders associated with or involving aberrant, pathologic, and/or abnormal xenobiotic metabolism signaling in the brain, neurons, and/or CNS and/or diseases or disorders thereof: PRKCE; EP300; PRKCZ; RXRA; MAPK1; NQO1; NCOR2; PIK3CA; ARNT; PRKCI; NFKB2; CAMK2A; PIK3CB; PPP2R1A; PIK3C3; MAPK8; PRKD1; ALDH1A1; MAPK3; NRIP1; KRAS; MAPK13; PRKCD; GSTP1; MAPK9; NOS2A; ABCB1; AHR; PPP2CA; FTL; NFE2L2; PIK3C2A; PPARGC1A; MAPK14; TNF; RAF1; CREBBP; MAP2K2; PIK3R1; PPP2R5C; MAP2K1; NFKB1; KEAP1; PRKCA; EIF2AK3; IL6; CYP1B1; HSP90AA1; in the case of diseases or disorders associated with or involving aberrant, pathologic, and/or abnormal SAPK/JNK signaling in the brain, neurons, and/or CNS and/or diseases or disorders thereof: PRKCE; IRAK1; PRKAA2; EIF2AK2; RAC1; ELK1; GRK6; MAPK1; GADD45A; RAC2; PLK1; AKT2; PIK3CA; FADD; CDK8; PIK3CB; PIK3C3; MAPK8; RIPK1; GNB2L1; IRS1; MAPK3; MAPK10; DAXX; KRAS; PRKCD; PRKAA1; MAPK9; CDK2; PIM1; PIK3C2A; TRAF2; TP53; LCK; MAP3K7; DYRKIA; MAP2K2; PIK3R1; MAP2K1; PAK3; CDC42; JUN; TTK; CSNK1A1; CRKL; BRAF; SGK; in the case of diseases or disorders associated with or involving aberrant, pathologic, and/or abnormal PPAr/RXR signaling in the brain, neurons, and/or CNS and/or diseases or disorders thereof: PRKAA2; EP300; INS; SMAD2; TRAF6; PPARA; FASN; RXRA; MAPK1; SMAD3; GNAS; IKBKB; NCOR2; ABCA1; GNAQ; NFKB2; MAP3K14; STAT5B; MAPK8; IRS1; MAPK3; KRAS; RELA; PRKAA1; PPARGC1A; NCOA3; MAPK14; INSR; RAF1; IKBKG; RELB; MAP3K7; CREBBP; MAP2K2; JAK2; CHUK; MAP2K1; NFKB1; TGFBR1; SMAD4; JUN; IL1R1; PRKCA; IL6; HSP90AA1; ADIPOQ; in the case of diseases or disorders associated with or involving aberrant, pathologic, and/or abnormal NF-kappaB signaling in the brain, neurons, and/or CNS and/or diseases or disorders thereof: IRAK1; EIF2AK2; EP300; INS; MYD88; PRKCZ; TRAF6; TBK1; AKT2; EGFR; IKBKB; PIK3CA; BTRC; NFKB2; MAP3K14; PIK3CB; PIK3C3; MAPK8; RIPK1; HDAC2; KRAS; RELA; PIK3C2A; TRAF2; TLR4; PDGFRB; TNF; INSR; LCK; IKBKG; RELB; MAP3K7; CREBBP; AKT1; PIK3R1; CHUK; PDGFRA; NFKB1; TLR2; BCL10; GSK3B; AKT3; TNFAIP3; IL1R1; in the case of diseases or disorders associated with or involving aberrant, pathologic, and/or abnormal neuregulin signaling in the brain, neurons, and/or CNS and/or diseases or disorders thereof: ERBB4; PRKCE; ITGAM; ITGA5; PTEN; PRKCZ; ELK1; MAPK1; PTPN11; AKT2; EGFR; ERBB2; PRKCI; CDKN1B; STAT5B; PRKD1; MAPK3; ITGA1; KRAS; PRKCD; STAT5A; SRC; ITGB7; RAF1; ITGB1; MAP2K2; ADAM17; AKT1; PIK3R1; PDPK1; MAP2K1; ITGB3; EREG; FRAP1; PSEN1; ITGA2; MYC; NRG1; CRKL; AKT3; PRKCA; HSP90AA1; RPS6KB1; in the case of diseases or disorders associated with or involving aberrant, pathologic, and/or abnormal wnt and beta catenin signaling in the brain, neurons, and/or CNS and/or diseases or disorders thereof: CD44; EP300; LRP6; DVL3; CSNK1E; GJA1; SMO; AKT2; PIN1; CDH1; BTRC; GNAQ; MARK2; PPP2R1A; WNT11; SRC; DKK1; PPP2CA; SOX6; SFRP2; ILK; LEF1; SOX9; TP53; MAP3K7; CREBBP; TCF7L2; AKT1; PPP2R5C; WNT5A; LRP5; CTNNB1; TGFBR1; CCND1; GSK3A; DVL1; APC; CDKN2A; MYC; CSNK1A1; GSK3B; AKT3; SOX2; in the case of diseases or disorders associated with or involving aberrant, pathologic, and/or abnormal insulin receptor signaling in the brain, neurons, and/or CNS and/or diseases or disorders thereof: PTEN; INS; EIF4E; PTPN1; PRKCZ; MAPK1; TSC1; PTPN11; AKT2; CBL; PIK3CA; PRKCI; PIK3CB; PIK3C3; MAPK8; IRS1; MAPK3; TSC2; KRAS; EIF4EBP1; SLC2A4; PIK3C2A; PPP1CC; INSR; RAF1; FYN; MAP2K2; JAK1; AKT1; JAK2; PIK3R1; PDPK1; MAP2K1; GSK3A; FRAP1; CRKL; GSK3B; AKT3; FOXOl; SGK; RPS6KB1; in the case of diseases or disorders associated with or involving aberrant, pathologic, and/or abnormal IL-6 signaling in the brain, neurons, and/or CNS and/or diseases or disorders thereof: HSPB1; TRAF6; MAPKAPK2; ELK1; MAPK1; PTPN11; IKBKB; FOS; NFKB2; MAP3K14; MAPK8; MAPK3; MAPK10; IL6ST; KRAS; MAPK13; IL6R; RELA; SOCS1; MAPK9; ABCB1; TRAF2; MAPK14; TNF; RAF1; IKBKG; RELB; MAP3K7; MAP2K2; IL8; JAK2; CHUK; STAT3; MAP2K1; NFKB1; CEBPB; JUN; ILIR1; SRF; IL6; in the case of diseases or disorders associated with or involving aberrant, pathologic, and/or abnormal IGF-1 signaling in the brain, neurons, and/or CNS and/or diseases or disorders thereof: IGF1; PRKCZ; ELK1; MAPK1; PTPN11; NEDD4; AKT2; PIK3CA; PRKCI; PTK2; FOS; PIK3CB; PIK3C3; MAPK8; IGF1R; IRS1; MAPK3; IGFBP7; KRAS; PIK3C2A; YWHAZ; PXN; RAF1; CASP9; MAP2K2; AKT1; PIK3R1; PDPK1; MAP2K1; IGFBP2; SFN; JUN; CYR61; AKT3; FOXOl; SRF; CTGF; RPS6KB1; in the case of diseases or disorders associated with or involving aberrant, pathologic, and/or abnormal NRF2-mediated oxidative stress response pathway regulation or signaling in the brain, neurons, and/or CNS and/or diseases or disorders thereof: PRKCE; EP300; SOD2; PRKCZ; MAPK1; SQSTM1; NQO1; PIK3CA; PRKCI; FOS; PIK3CB; PIK3C3; MAPK8; PRKD1; MAPK3; KRAS; PRKCD; GSTP1; MAPK9; FTL; NFE2L2; PIK3C2A; MAPK14; RAF1; MAP3K7; CREBBP; MAP2K2; AKT1; PIK3R1; MAP2K1; PPIB; JUN; KEAP1; GSK3B; ATF4; PRKCA; EIF2AK3; HSP90AA1; PRDX1; in the case of diseases or disorders associated with or involving aberrant, pathologic, and/or abnormal PPAR (e.g. PPAR alpha, PPAR beta, PPAR delta, and/or PPAR gamma) regulation or signaling in the brain, neurons, and/or CNS and/or diseases or disorders thereof: EP300; INS; TRAF6; PPARA; RXRA; MAPK1; IKBKB; NCOR2; FOS; NFKB2; MAP3K14; STAT5B; MAPK3; NRIP1; KRAS; PPARG; RELA; STAT5A; TRAF2; PPARGC1A; PDGFRB; TNF; INSR; RAF1; IKBKG; RELB; MAP3K7; CREBBP; MAP2K2; CHUK; PDGFRA; MAP2K1; NFKB1; JUN; ILIR1; HSP90AA1; in the case of diseases or disorders associated with or involving aberrant, pathologic, and/or abnormal Fc Epsilon RI regulation or signaling in the brain, neurons, and/or CNS and/or diseases or disorders thereof: PRKCE; RAC1; PRKCZ; LYN; MAPK1; RAC2; PTPN11; AKT2; PIK3CA; SYK; PRKCI; PIK3CB; PIK3C3; MAPK8; PRKD1; MAPK3; MAPK10; KRAS; MAPK13; PRKCD; MAPK9; PIK3C2A; BTK; MAPK14; TNF; RAF1; FYN; MAP2K2; AKT1; PIK3R1; PDPK1; MAP2K1; AKT3; VAV3; PRKCA; in the case of diseases or disorders associated with or involving aberrant, pathologic, and/or abnormal G-protein coupled receptor regulation or signaling in the brain, neurons, and/or CNS and/or diseases or disorders thereof: PRKCE; RAPlA; RGS16; MAPK1; GNAS; AKT2; IKBKB; PIK3CA; CREB1; GNAQ; NFKB2; CAMK2A; PIK3CB; PIK3C3; MAPK3; KRAS; RELA; SRC; PIK3C2A; RAF1; IKBKG; RELB; FYN; MAP2K2; AKT1; PIK3R1; CHUK; PDPK1; STAT3; MAP2K1; NFKB1; BRAF; ATF4; AKT3; PRKCA; in the case of diseases or disorders associated with or involving aberrant, pathologic, and/or abnormal inositol phosphate metabolism regulation or signaling in the brain, neurons, and/or CNS and/or diseases or disorders thereof: PRKCE; IRAK1; PRKAA2; EIF2AK2; PTEN; GRK6; MAPK1; PLK1; AKT2; PIK3CA; CDK8; PIK3CB; PIK3C3; MAPK8; MAPK3; PRKCD; PRKAA1; MAPK9; CDK2; PIM1; PIK3C2A; DYRKIA; MAP2K2; PIP5K1A; PIK3R1; MAP2K1; PAK3; ATM; TTK; CSNK1A1; BRAF; SGK; in the case of diseases or disorders associated with or involving aberrant, pathologic, and/or abnormal PDGF regulation or signaling in the brain, neurons, and/or CNS and/or diseases or disorders thereof: EIF2AK2; ELK1; ABL2; MAPK1; PIK3CA; FOS; PIK3CB; PIK3C3; MAPK8; CAV1; ABL1; MAPK3; KRAS; SRC; PIK3C2A; PDGFRB; RAF1; MAP2K2; JAK1; JAK2; PIK3R1; PDGFRA; STAT3; SPHK1; MAP2K1; MYC; JUN; CRKL; PRKCA; SRF; STAT1; SPHK2; in the case of diseases or disorders associated with involving aberrant, pathologic, and/or abnormal VEGF regulation or signaling in the brain, neurons, and/or CNS and/or diseases or disorders thereof: ACTN4; ROCK1; KDR; FLT1; ROCK2; MAPK1; PGF; AKT2; PIK3CA; ARNT; PTK2; BCL2; PIK3CB; PIK3C3; BCL2L1; MAPK3; KRAS; HIF1A; NOS3; PIK3C2A; PXN; RAF1; MAP2K2; ELAVL1; AKT1; PIK3R1; MAP2K1; SFN; VEGFA; AKT3; FOXOl; PRKCA; in the case of diseases or disorders associated with or involving aberrant, pathologic, and/or abnormal natural killer cell regulation or signaling in the brain, neurons, and/or CNS and/or diseases or disorders thereof: PRKCE; RAC1; PRKCZ; MAPK1; RAC2; PTPN11; KIR2DL3; AKT2; PIK3CA; SYK; PRKCI; PIK3CB; PIK3C3; PRKD1; MAPK3; KRAS; PRKCD; PTPN6; PIK3C2A; LCK; RAF1; FYN; MAP2K2; PAK4; AKT1; PIK3R1; MAP2K1; PAK3; AKT3; VAV3; PRKCA; in the case of diseases or disorders associated with or involving aberrant, pathologic, and/or abnormal cell cycle G1/S checkpoint regulation or signaling in the brain, neurons, and/or CNS and/or diseases or disorders thereof: HDAC4; SMAD3; SUV39H1; HDAC5; CDKN1B; BTRC; ATR; ABL1; E2F1; HDAC2; HDAC7A; RBl; HDAC11; HDAC9; CDK2; E2F2; HDAC3; TP53; CDKN1A; CCND1; E2F4; ATM; RBL2; SMAD4; CDKN2A; MYC; NRG1; GSK3B; RBL1; HDAC6; in the case of diseases or disorders associated with or involving aberrant, pathologic, and/or abnormal T-cell receptor regulation or signaling in the brain, neurons, and/or CNS and/or diseases or disorders thereof: RAC1; ELK1; MAPK1; IKBKB; CBL; PIK3CA; FOS; NFKB2; PIK3CB; PIK3C3; MAPK8; MAPK3; KRAS; RELA; PIK3C2A; BTK; LCK; RAF1; IKBKG; RELB; FYN; MAP2K2; PIK3R1; CHUK; MAP2K1; NFKB1; ITK; BCL10; JUN; VAV3; in the case of diseases or disorders associated with or involving aberrant, pathologic, and/or abnormal death receptor regulation or signaling in the brain, neurons, and/or CNS and/or diseases or disorders thereof: CRADD; HSPB1; BID; BIRC4; TBK1; IKBKB; FADD; FAS; NFKB2; BCL2; MAP3K14; MAPK8; RIPK1; CASP8; DAXX; TNFRSF10B; RELA; TRAF2; TNF; IKBKG; RELB; CASP9; CHUK; APAF1; NFKB1; CASP2; BIRC2; CASP3; BIRC3; in the case of diseases or disorders associated with or involving aberrant, pathologic, and/or FGF regulation or signaling in the brain, neurons, and/or CNS and/or diseases or disorders thereof: RAC1; FGFR1; MET; MAPKAPK2; MAPK1; PTPN11; AKT2; PIK3CA; CREB1; PIK3CB; PIK3C3; MAPK8; MAPK3; MAPK13; PTPN6; PIK3C2A; MAPK14; RAF1; AKT1; PIK3R1; STAT3; MAP2K1; FGFR4; CRKL; ATF4; AKT3; PRKCA; HGF; in the case of diseases or disorders associated with or involving aberrant, pathologic, and/or GM-CSF regulation or signaling in the brain, neurons, and/or CNS and/or diseases or disorders thereof: LYN; ELK1; MAPK1; PTPN11; AKT2; PIK3CA; CAMK2A; STAT5B; PIK3CB; PIK3C3; GNB2L1; BCL2L1; MAPK3; ETS1; KRAS; RUNX1; PIM1; PIK3C2A; RAF1; MAP2K2; AKT1; JAK2; PIK3R1; STAT3; MAP2K1; CCND1; AKT3; STAT1; in the case of diseases or disorders associated with or involving aberrant, pathologic, and/or amyotrophic lateral sclerosis regulation or signaling in the brain, neurons, and/or CNS and/or diseases or disorders thereof: BID; IGF1; RAC1; BIRC4; PGF; CAPNS1; CAPN2; PIK3CA; BCL2; PIK3CB; PIK3C3; BCL2L1; CAPN1; PIK3C2A; TP53; CASP9; PIK3R1; RAB5A; CASP1; APAF1; VEGFA; BIRC2; BAX; AKT3; CASP3; BIRC3; in the case of diseases or disorders associated with or involving aberrant, pathologic, and/or JAK/Stat regulation or signaling in the brain, neurons, and/or CNS and/or diseases or disorders thereof: PTPN1; MAPK1; PTPN11; AKT2; PIK3CA; STAT5B; PIK3CB; PIK3C3; MAPK3; KRAS; SOCS1; STAT5A; PTPN6; PIK3C2A; RAF1; CDKN1A; MAP2K2; JAK1; AKT1; JAK2; PIK3R1; STAT3; MAP2K1; FRAP1; AKT3; STAT1; in the case of diseases or disorders associated with or involving aberrant, pathologic, and/or nicotinate and nicotinamide metabolism regulation or signaling in the brain, neurons, and/or CNS and/or diseases or disorders thereof: PRKCE; IRAK1; PRKAA2; EIF2AK2; GRK6; MAPK1; PLK1; AKT2; CDK8; MAPK8; MAPK3; PRKCD; PRKAA1; PBEF1; MAPK9; CDK2; PIM1; DYRKIA; MAP2K2; MAP2K1; PAK3; NT5E; TTK; CSNK1A1; BRAF; SGK; in the case of diseases or disorders associated with or involving aberrant, pathologic, and/or chemokine signaling regulation or signaling in the brain, neurons, and/or CNS and/or diseases or disorders thereof: CXCR4; ROCK2; MAPK1; PTK2; FOS; CFL1; GNAQ; CAMK2A; CXCL12; MAPK8; MAPK3; KRAS; MAPK13; RHOA; CCR3; SRC; PPP1CC; MAPK14; NOX1; RAF1; MAP2K2; MAP2K1; JUN; CCL2; PRKCA; in the case of diseases or disorders associated with or involving aberrant, pathologic, and/or IL-2 signaling regulation or signaling in the brain, neurons, and/or CNS and/or diseases or disorders thereof: ELK1; MAPK1; PTPN11; AKT2; PIK3CA; SYK; FOS; STAT5B; PIK3CB; PIK3C3; MAPK8; MAPK3; KRAS; SOCS1; STAT5A; PIK3C2A; LCK; RAFI; MAP2K2; JAK1; AKT1; PIK3R1; MAP2K1; JUN; AKT3; in the case of diseases or disorders associated with or involving synaptic long term depression in the brain, neurons, and/or CNS and/or diseases or disorders thereof: PRKCE; IGF1; PRKCZ; PRDX6; LYN; MAPK1; GNAS; PRKCI; GNAQ; PPP2R1A; IGF1R; PRKD1; MAPK3; KRAS; GRN; PRKCD; NOS3; NOS2A; PPP2CA; YWHAZ; RAF1; MAP2K2; PPP2R5C; MAP2K1; PRKCA; in the case of diseases or disorders associated with or involving aberrant, pathologic, and/or estrogen receptor regulation or signaling in the brain, neurons, and/or CNS and/or diseases or disorders thereof: TAF4B; EP300; CARM1; PCAF; MAPK1; NCOR2; SMARCA4; MAPK3; NRIP1; KRAS; SRC; NR3C1; HDAC3; PPARGC1A; RBM9; NCOA3; RAF1; CREBBP; MAP2K2; NCOA2; MAP2K1; PRKDC; ESR1; ESR2; in the case of diseases or disorders associated with or involving aberrant, pathologic, and/or protein ubiquitination pathway activity, regulation, and/or signaling in the brain, neurons, and/or CNS and/or diseases or disorders thereof: TRAF6; SMURF1; BIRC4; BRCA1; UCHL1; NEDD4; CBL; UBE2I; BTRC; HSPA5; USP7; USP10; FBXW7; USP9X; STUB1; USP22; B2M; BIRC2; PARK2; USP8; USP1; VHL; HSP90AA1; BIRC3; in the case of diseases or disorders associated with or involving aberrant, pathologic, and/or IL-10 regulation or signaling in the brain, neurons, and/or CNS and/or diseases or disorders thereof: TRAF6; CCR1; ELK1; IKBKB; SP1; FOS; NFKB2; MAP3K14; MAPK8; MAPK13; RELA; MAPK14; TNF; IKBKG; RELB; MAP3K7; JAK1; CHUK; STAT3; NFKB1; JUN; IL1R1; IL6; in the case of diseases or disorders associated with or involving aberrant, pathologic, and/or Vitamin D receptor (VDR) and/or RXR regulation or signaling in the brain, neurons, and/or CNS and/or diseases or disorders thereof: PRKCE; EP300; PRKCZ; RXRA; GADD45A; HES1; NCOR2; SP1; PRKCI; CDKN1B; PRKD1; PRKCD; RUNX2; KLF4; YY1; NCOA3; CDKN1A; NCOA2; SPP1; LRP5; CEBPB; FOXOl; PRKCA; in the case of diseases or disorders associated with or involving aberrant, pathologic, and/or TGF-beta regulation or signaling in the brain, neurons, and/or CNS and/or diseases or disorders thereof: EP300; SMAD2; SMURF1; MAPK1; SMAD3; SMAD1; FOS; MAPK8; MAPK3; KRAS; MAPK9; RUNX2; SERPINE1; RAF1; MAP3K7; CREBBP; MAP2K2; MAP2K1; TGFBR1; SMAD4; JUN; SMAD5; in the case of diseases or disorders associated with or involving aberrant, pathologic, and/or Toll-like Receptor activity, regulation, and/or signaling in the brain, neurons, and/or CNS and/or diseases or disorders thereof: IRAK1; EIF2AK2; MYD88; TRAF6; PPARA; ELK1; IKBKB; FOS; NFKB2; MAP3K14; MAPK8; MAPK13; RELA; TLR4; MAPK14; IKBKG; RELB; MAP3K7; CHUK; NFKB1; TLR2; JUN; in the case of diseases or disorders associated with or involving aberrant, pathologic, and/or p38 MAPK activity, regulation, and/or signaling in the brain, neurons, and/or CNS and/or diseases or disorders thereof: HSPB1; IRAK1; TRAF6; MAPKAPK2; ELK1; FADD; FAS; CREB1; DDIT3; RPS6KA4; DAXX; MAPK13; TRAF2; MAPK14; TNF; MAP3K7; TGFBR1; MYC; ATF4; IL1R1; SRF; STAT1; in the case of diseases or disorders associated with or involving aberrant, pathologic, and/or neurotrophin/TRK activity, regulation, and/or signaling in the brain, neurons, and/or CNS and/or diseases or disorders thereof: NTRK2; MAPK1; PTPN11; PIK3CA; CREB1; FOS; PIK3CB; PIK3C3; MAPK8; MAPK3; KRAS; PIK3C2A; RAF1; MAP2K2; AKT1; PIK3R1; PDPK1; MAP2K1; CDC42; JUN; ATF4; in the case of diseases or disorders associated with or involving aberrant, pathologic, and/or FXR and/or RXR activity, regulation, and/or signaling in the brain, neurons, and/or CNS and/or diseases or disorders thereof: INS; PPARA; FASN; RXRA; AKT2; SDC1; MAPK8; APOB; MAPK10; PPARG; MTTP; MAPK9; PPARGC1A; TNF; CREBBP; AKT1; SREBF1; FGFR4; AKT3; FOXOl; in the case of diseases or disorders associated with or involving aberrant, pathologic, and/or synaptic long term potentiation in the brain, neurons, and/or CNS and/or diseases or disorders thereof: PRKCE; RAPlA; EP300; PRKCZ; MAPK1; CREB1; PRKCI; GNAQ; CAMK2A; PRKD1; MAPK3; KRAS; PRKCD; PPP1CC; RAF1; CREBBP; MAP2K2; MAP2K1; ATF4; PRKCA; in the case of diseases or disorders associated with or involving aberrant, pathologic, and/or calcium regulation and/or signaling in the brain, neurons, and/or CNS and/or diseases or disorders thereof: RAPlA; EP300; HDAC4; MAPK1; HDAC5; CREB1; CAMK2A; MYH9; MAPK3; HDAC2; HDAC7A; HDAC11; HDAC9; HDAC3; CREBBP; CALR; CAMKK2; ATF4; HDAC6; in the case of diseases or disorders associated with or involving aberrant, pathologic, and/or EGF or EGFR regulation and/or signaling in the brain, neurons, and/or CNS and/or diseases or disorders thereof: ELK1; MAPK1; EGFR; PIK3CA; FOS; PIK3CB; PIK3C3; MAPK8; MAPK3; PIK3C2A; RAF1; JAK1; PIK3R1; STAT3; MAP2K1; JUN; PRKCA; SRF; STAT1; in the case of diseases or disorders associated with or involving aberrant, pathologic, and/or LPS/IL-1 mediated inhibition of RXR function, regulation and/or signaling in the brain, neurons, and/or CNS and/or diseases or disorders thereof: IRAK1; MYD88; TRAF6; PPARA; RXRA; ABCA1; MAPK8; ALDH1A1; GSTP1; MAPK9; ABCB1; TRAF2; TLR4; TNF; MAP3K7; NR1H2; SREBF1; JUN; IL1R1; in the case of diseases or disorders associated with or involving aberrant, pathologic, and/or LXR/RXR function, regulation and/or signaling in the brain, neurons, and/or CNS and/or diseases or disorders thereof: FASN; RXRA; NCOR2; ABCA1; NFKB2; IRF3; RELA; NOS2A; TLR4; TNF; RELB; LDLR; NR1H2; NFKB1; SREBF1; IL1R1; CCL2; IL6; MMP9; in the case of diseases or disorders associated with or involving aberrant, pathologic, and/or amyloid processing in the brain, neurons, and/or CNS and/or diseases or disorders thereof: PRKCE; CSNK1E; MAPK1; CAPNS1; AKT2; CAPN2; CAPN1; MAPK3; MAPK13; MAPT; MAPK14; AKT1; PSEN1; CSNK1A1; GSK3B; AKT3; APP; in the case of diseases or disorders associated with or involving aberrant, pathologic, and/or abnormal IL-4 activity, signaling, and/or regulation in the brain, neurons, and/or CNS and/or diseases or disorders thereof: AKT2; PIK3CA; PIK3CB; PIK3C3; IRS1; KRAS; SOCS1; PTPN6; NR3C1; PIK3C2A; JAK1; AKT1; JAK2; PIK3R1; FRAP1; AKT3; RPS6KB1; in the case of diseases or disorders associated with or involving aberrant, pathologic, and/or abnormal cell cycle: G2/M DNA damage checkpoint regulation activity, signaling, and/or regulation in the brain, neurons, and/or CNS and/or diseases or disorders thereof: EP300; PCAF; BRCA1; GADD45A; PLK1; BTRC; CHEK1; ATR; CHEK2; YWHAZ; TP53; CDKN1A; PRKDC; ATM; SFN; CDKN2A; in the case of diseases or disorders associated with or involving aberrant, pathologic, and/or abnormal purine metabolism signaling thereof, and/or regulation thereof in the brain, neurons, and/or CNS and/or diseases or disorders thereof: NME2; SMARCA4; MYH9; RRM2; ADAR; EIF2AK4; PKM2; ENTPD1; RAD51; RRM2B; TJP2; RAD51C; NT5E; POLD1; NME1; in the case of diseases or disorders associated with or involving aberrant, pathologic, and/or abnormal cAMP-mediated signaling and/or regulation in the brain, neurons, and/or CNS and/or diseases or disorders thereof: RAPlA; MAPK1; GNAS; CREB1; CAMK2A; MAPK3; SRC; RAF1; MAP2K2; STAT3; MAP2K1; BRAF; ATF4; in the case of diseases or disorders associated with or involving aberrant, pathologic, and/or abnormal mitochondrial function in the brain, neurons, and/or CNS and/or diseases or disorders thereof: SOD2; MAPK8; CASP8; MAPK10; MAPK9; CASP9; PARK7; PSEN1; PARK2; APP; CASP3; AIF; CytC; SMAC (Diablo); Aifm-1; Aifm-2; in the case of diseases or disorders associated with or involving aberrant, pathologic, and/or abnormal notch signaling and/or regulation in the brain, neurons, and/or CNS and/or diseases or disorders thereof: HES1; JAG1; NUMB; NOTCH4; ADAM17; NOTCH2; PSEN1; NOTCH3; NOTCH1; DLL4; in the case of diseases or disorders associated with or involving aberrant, pathologic, and/or abnormal endoplasmic reticulum stress pathway activity, signaling, and/or regulation in the brain, neurons, and/or CNS and/or diseases or disorders thereof: HSPA5; MAPK8; XBP1; TRAF2; ATF6; CASP9; ATF4; EIF2AK3; CASP3; in the case of diseases or disorders associated with or involving aberrant, pathologic, and/or abnormal pyrimidine metabolism, signaling thereof, and/or regulation thereof in the brain, neurons, and/or CNS and/or diseases or disorders thereof: NME2; AICDA; RRM2; EIF2AK4; ENTPD1; RRM2B; NT5E; POLD1; NME1; in the case of diseases or disorders associated with or involving aberrant, pathologic, and/or abnormal Parkinson's signaling in the brain, neurons, and/or CNS and/or diseases or disorders thereof: UCHL1; MAPK8; MAPK13; MAPK14; CASP9; PARK7; PARK2; CASP3; in the case of diseases or disorders associated with or involving aberrant, pathologic, and/or abnormal Glycolysis/Gluconeogenesis activity, signaling, and/or regulation in the brain, neurons, and/or CNS and/or diseases or disorders thereof: HK2; GCK; GPI; ALDH1A1; PKM2; LDHA; HK1; in the case of diseases or disorders associated with or involving aberrant, pathologic, and/or abnormal interferon activity, signaling, and/or regulation in the brain, neurons, and/or CNS and/or diseases or disorders thereof: IRF1; SOCS1; JAK1; JAK2; IFITM1; STAT1; IFIT3; in the case of diseases or disorders associated with or involving aberrant, pathologic, and/or abnormal sonic the hedgehog activity, signaling, and/or regulation in the brain, neurons, and/or CNS and/or diseases or disorders thereof: ARRB2; SMO; GLI2; DYRKIA; GLI1; GSK3B; DYRK1B; in the case of diseases or disorders associated with or involving aberrant, pathologic, and/or abnormal glycerophospholipid metabolism, signaling thereof, and/or regulation thereof in the brain, neurons, and/or CNS and/or diseases or disorders thereof: PLD1; GRN; GPAM; YWHAZ; SPHK1; SPHK2; in the case of diseases or disorders associated with or involving aberrant, pathologic, and/or abnormal phospholipid degradation, signaling thereof, and/or regulation thereof in the brain, neurons, and/or CNS and/or diseases or disorders thereof: PRDX6; PLD1; GRN; YWHAZ; SPHK1; SPHK2; in the case of diseases or disorders associated with or involving aberrant, pathologic, and/or abnormal tryptophan metabolism, signaling thereof, and/or regulation thereof in the brain, neurons, and/or CNS and/or diseases or disorders thereof: SIAH2; PRMT5; NEDD4; ALDH1A1; CYP1B1; SIAH1; in the case of diseases or disorders associated with or involving aberrant, pathologic, and/or abnormal lysine degradation, signaling thereof, and/or regulation thereof in the brain, neurons, and/or CNS and/or diseases or disorders thereof: SUV39H1; EHMT2; NSD1; SETD7; PPP2R5C; in the case of diseases or disorders associated with or involving aberrant, pathologic, and/or abnormal nucleotide excision repair pathway activity, signaling thereof, and/or regulation thereof in the brain, neurons, and/or CNS and/or diseases or disorders thereof: ERCC5; ERCC4; XPA; XPC; ERCC1; in the case of diseases or disorders associated with or involving aberrant, pathologic, and/or abnormal nucleotide starch and sucrose metabolism, signaling thereof, and/or regulation thereof in the brain, neurons, and/or CNS and/or diseases or disorders thereof: UCHL1; HK2; GCK; GPI; HK1; in the case of diseases or disorders associated with or involving aberrant, pathologic, and/or abnormal aminosugars metabolism, signaling thereof, and/or regulation thereof in the brain, neurons, and/or CNS and/or diseases or disorders thereof: NQO1; HK2; GCK; HK1; in the case of diseases or disorders associated with or involving aberrant, pathologic, and/or abnormal arachidonic acid metabolism, signaling thereof, and/or regulation thereof in the brain, neurons, and/or CNS and/or diseases or disorders thereof: PRDX6; GRN; YWHAZ; CYP1B1; in the case of diseases or disorders associated with or involving aberrant, pathologic, and/or abnormal circadian rhythm signaling and/or regulation in the brain, neurons, and/or CNS and/or diseases or disorders thereof: CSNK1E; CREB1; ATF4; NR1D1; in the case of diseases or disorders associated with or involving aberrant, pathologic, and/or coagulation system activity signaling and/or regulation in the brain, neurons, and/or CNS and/or diseases or disorders thereof: BDKRB1; F2R; SERPINE1; F3; a PAR (e.g. PAR1, PAR2, etc.) PLC, aPC; in the case of diseases or disorders associated with or involving aberrant, pathologic, and/or abnormal dopamine receptor signaling and/or regulation in the brain, neurons, and/or CNS and/or diseases or disorders thereof: PPP2R1A; PPP2CA; PPP1CC; PPP2R5C; in the case of diseases or disorders associated with or involving aberrant, pathologic, and/or abnormal Glutathione Metabolism signaling and/or regulation in the brain, neurons, and/or CNS and/or diseases or disorders thereof: IDH2; GSTP1; ANPEP; IDH1; in the case of diseases or disorders associated with or involving aberrant, pathologic, and/or abnormal Glycerolipid Metabolism signaling and/or regulation in the brain, neurons, and/or CNS and/or diseases or disorders thereof: ALDH1A1; GPAM; SPHK1; SPHK2; in the case of diseases or disorders associated with or involving aberrant, pathologic, and/or abnormal Linoleic Acid Metabolism signaling and/or regulation in the brain, neurons, and/or CNS and/or diseases or disorders thereof: PRDX6; GRN; YWHAZ; CYP1B1; in the case of diseases or disorders associated with or involving aberrant, pathologic, and/or abnormal Methionine Metabolism signaling and/or regulation in the brain, neurons, and/or CNS and/or diseases or disorders thereof: DNMT1; DNMT3B; AHCY; DNMT3A; in the case of diseases or disorders associated with or involving aberrant, pathologic, and/or abnormal Pyruvate Metabolism signaling and/or regulation in the brain, neurons, and/or CNS and/or diseases or disorders thereof: GLO1; ALDH1A1; PKM2; LDHA; in the case of diseases or disorders associated with or involving aberrant, pathologic, and/or abnormal Arginine and Proline Metabolism signaling and/or regulation in the brain, neurons, and/or CNS and/or diseases or disorders thereof: ALDH1A1; NOS3; NOS2A; in the case of diseases or disorders associated with or involving aberrant, pathologic, and/or abnormal Eicosanoid signaling and/or regulation in the brain, neurons, and/or CNS and/or diseases or disorders thereof: PRDX6; GRN; YWHAZ; in the case of diseases or disorders associated with or involving aberrant, pathologic, and/or abnormal fructose and mannose metabolism signaling and/or regulation in the brain, neurons, and/or CNS and/or diseases or disorders thereof: HK2; GCK; HK1; in the case of diseases or disorders associated with or involving aberrant, pathologic, and/or abnormal antigen presentation pathway activity, signaling and/or regulation in the brain, neurons, and/or CNS and/or diseases or disorders thereof: CALR; B2M; in the case of diseases or disorders associated with or involving aberrant, pathologic, and/or abnormal steroid biosynthesis in the brain, neurons, and/or CNS and/or diseases or disorders thereof: NQO1; DHCR7; in the case of diseases or disorders associated with or involving aberrant, pathologic, and/or abnormal butanoate metabolism in the brain, neurons, and/or CNS and/or diseases or disorders thereof: ALDH1A1; NLGN1; in the case of diseases or disorders associated with or involving an aberrant, pathologic, and/or abnormal citrate cycle in the brain, neurons, and/or CNS and/or diseases or disorders thereof: IDH2; IDH1; in the case of diseases or disorders associated with or involving aberrant, pathologic, and/or abnormal fatty acid metabolism in the brain, neurons, and/or CNS and/or diseases or disorders thereof: ALDH1A1; CYP1B1; in the case of diseases or disorders associated with or involving aberrant, pathologic, and/or abnormal Glycerophospholipid metabolism in the brain, neurons, and/or CNS and/or diseases or disorders thereof: PRDX6; CHKA; in the case of diseases or disorders associated with or involving aberrant, pathologic, and/or abnormal histidine metabolism in the brain, neurons, and/or CNS and/or diseases or disorders thereof: PRMT5; ALDH1A1; in the case of diseases or disorders associated with or involving aberrant, pathologic, and/or abnormal inositol metabolism in the brain, neurons, and/or CNS and/or diseases or disorders thereof: ERO1L; APEX1; in the case of diseases or disorders associated with or involving aberrant, pathologic, and/or abnormal Phenylalanine metabolism in the brain, neurons, and/or CNS and/or diseases or disorders thereof: PRDX6; PRDX1; in the case of diseases or disorders associated with or involving aberrant, pathologic, and/or abnormal Seleno amino acid metabolism in the brain, neurons, and/or CNS and/or diseases or disorders thereof: PRMT5; AHCY; in the case of diseases or disorders associated with or involving aberrant, pathologic, and/or abnormal Sphingolipid metabolism in the brain, neurons, and/or CNS and/or diseases or disorders thereof: SPHK1; SPHK2; in the case of diseases or disorders associated with or involving aberrant, pathologic, and/or abnormal Aminophosphonate metabolism in the brain, neurons, and/or CNS and/or diseases or disorders thereof: PRMT5; in the case of diseases or disorders associated with or involving aberrant, pathologic, and/or abnormal androgen and/or estrogen metabolism in the brain, neurons, and/or CNS and/or diseases or disorders thereof: PRMT5; in the case of diseases or disorders associated with or involving aberrant, pathologic, and/or abnormal Ascorbate and Aldarate metabolism in the brain, neurons, and/or CNS and/or diseases or disorders thereof: ALDH1A1; in the case of diseases or disorders associated with or involving aberrant, pathologic, and/or abnormal Cysteine Metabolism in the brain, neurons, and/or CNS and/or diseases or disorders thereof: LDHA; in the case of diseases or disorders associated with or involving aberrant, pathologic, and/or abnormal fatty acid biosynthesis in the brain, neurons, and/or CNS and/or diseases or disorders thereof: FASN; in the case of diseases or disorders associated with or involving aberrant, pathologic, and/or abnormal glutamate receptor signaling in the brain, neurons, and/or CNS and/or diseases or disorders thereof: GNB2L1; in the case of diseases or disorders associated with or involving aberrant, pathologic, and/or abnormal Pentose Phosphate pathway in the brain, neurons, and/or CNS and/or diseases or disorders thereof: GPI; in the case of diseases or disorders associated with or involving aberrant, pathologic, and/or abnormal retinol metabolism in the brain, neurons, and/or CNS and/or diseases or disorders thereof: ALDH1A1; in the case of diseases or disorders associated with or involving aberrant, pathologic, and/or abnormal Pentose and Glucuronate interconversions in the brain, neurons, and/or CNS and/or diseases or disorders thereof: UCHL1; in the case of diseases or disorders associated with or involving aberrant, pathologic, and/or abnormal Riboflavin Metabolism in the brain, neurons, and/or CNS and/or diseases or disorders thereof: TYR; in the case of diseases or disorders associated with or involving aberrant, pathologic, and/or abnormal Tyrosine Metabolism in the brain, neurons, and/or CNS and/or diseases or disorders thereof: PRMT5, TYR; in the case of diseases or disorders associated with or involving aberrant, pathologic, and/or abnormal Ubiquinone biosynthesis in the brain, neurons, and/or CNS and/or diseases or disorders thereof: PRMT5; in the case of diseases or disorders associated with or involving aberrant, pathologic, and/or abnormal Valine, leucine and isoleucine degradation in the brain, neurons, and/or CNS and/or diseases or disorders thereof: ALDH1A1; in the case of diseases or disorders associated with or involving aberrant, pathologic, and/or abnormal glycine, serine, and threonine metabolism in the brain, neurons, and/or CNS and/or diseases or disorders thereof: CHKA; in the case of diseases or disorders associated with or involving aberrant, pathologic, and/or abnormal lysine degradation in the brain, neurons, and/or CNS and/or diseases or disorders thereof: ALDH1A1; in the case of diseases or disorders associated with or involving aberrant, pathologic, and/or abnormal pain or pain signaling or pain signal generation in the brain, neurons, and/or CNS and/or diseases or disorders thereof: TRPM7; TRPC5; TRPC6; TRPC1; Cnr1; cnr2; Grk2; Trpa1; Pomc; Cgrp; Crf; Pka; Era; Nr2b; TRPM5; Prkaca; Prkacb; Prkarla; Prkar2a; in the case of diseases or disorders associated with or involving aberrant, pathologic, and/or abnormal brain, neuron, and/or CNS development and/or diseases or disorders thereof: BMP-4; Chordin (Chrd); Noggin (Nog); WNT (Wnt2; Wnt2b; Wnt3a; Wnt4; Wnt5a; Wnt6; Wnt7b; Wnt8b; Wnt9a; Wnt9b; Wnt10a; Wnt10b; Wnt16); beta-catenin; Dkk-1; Frizzled related proteins; Otx-2; Gbx2; FGF-8; Reelin; Dabl; unc-86 (Pou4f1 or Brn3a); Numb; Reln; in the case of diseases or disorders associated with or involving prion disorders of or in the brain, neuron, and/or CNS and/or diseases or disorders thereof: Prp; in the case of substance or activity additions involving activities of the brain, neuron, and/or CNS: Prkce (alcohol); Drd2; Drd4; ABAT (alcohol); GRIA2; Grm5; Grin1; Htrlb; Grin2a; Drd3; Pdyn; Gria1 (alcohol); in the case of diseases or disorders associated with or involving PI3K/AKT signaling and/or regulation thereof in the brain, neuron, and/or CNS and/or diseases or disorders thereof: PRKCE; ITGAM; ITGA5; IRAK1; PRKAA2; EIF2AK2; PTEN; EIF4E; PRKCZ; GRK6; MAPK1; TSC1; PLK1; AKT2; IKBKB; PIK3CA; CDK8; CDKN1B; NFKB2; BCL2; PIK3CB; PPP2R1A; MAPK8; BCL2L1; MAPK3; TSC2; ITGA1; KRAS; EIF4EBP1; RELA; PRKCD; NOS3; PRKAA1; MAPK9; CDK2; PPP2CA; PIM1; ITGB7; YWHAZ; ILK; TP53; RAF1; IKBKG; RELB; DYRKIA; CDKN1A; ITGB1; MAP2K2; JAK1; AKT1; JAK2; PIK3R1; CHUK; PDPK1; PPP2R5C; CTNNB1; MAP2K1; NFKB1; PAK3; ITGB3; CCND1; GSK3A; FRAP1; SFN; ITGA2; TTK; CSNK1A1; BRAF; GSK3B; AKT3; FOXOl; SGK; HSP90AA1; RPS6KB1; in the case of diseases or disorders associated with or involving ERK/MAPK signaling and/or regulation thereof in the brain, neuron, and/or CNS and/or diseases or disorders thereof: PRKCE; ITGAM; ITGA5; HSPB1; IRAK1; PRKAA2; EIF2AK2; RAC1; RAPlA; TLN1; EIF4E; ELK1; GRK6; MAPK1; RAC2; PLK1; AKT2; PIK3CA; CDK8; CREB1; PRKCI; PTK2; FOS; RPS6KA4; PIK3CB; PPP2R1A; PIK3C3; MAPK8; MAPK3; ITGA1; ETS1; KRAS; MYCN; EIF4EBP1; PPARG; PRKCD; PRKAA1; MAPK9; SRC; CDK2; PPP2CA; PIM1; PIK3C2A; ITGB7; YWHAZ; PPP1CC; KSR1; PXN; RAF1; FYN; DYRKIA; ITGB1; MAP2K2; PAK4; PIK3R1; STAT3; PPP2R5C; MAP2K1; PAK3; ITGB3; ESR1; ITGA2; MYC; TTK; CSNK1A1; CRKL; BRAF; ATF4; PRKCA; SRF; STAT1; SGK; in the case of diseases or disorders associated with or involving glucocorticoid receptor signaling and/or regulation thereof in the brain, neuron, and/or CNS and/or diseases or disorders thereof: RAC1; TAF4B; EP300; SMAD2; TRAF6; PCAF; ELK1; MAPK1; SMAD3; AKT2; IKBKB; NCOR2; UBE2I; PIK3CA; CREB1; FOS; HSPA5; NFKB2; BCL2; MAP3K14; STAT5B; PIK3CB; PIK3C3; MAPK8; BCL2L1; MAPK3; TSC22D3; MAPK10; NRIP1; KRAS; MAPK13; RELA; STAT5A; MAPK9; NOS2A; PBX1; NR3C1; PIK3C2A; CDKN1C; TRAF2; SERPINE1; NCOA3; MAPK14; TNF; RAF1; IKBKG; MAP3K7; CREBBP; CDKN1A; MAP2K2; JAK1; IL8; NCOA2; AKT1; JAK2; PIK3R1; CHUK; STAT3; MAP2K1; NFKB1; TGFBR1; ESR1; SMAD4; CEBPB; JUN; AR; AKT3; CCL2; MMP1; STAT1; IL6; HSP90AA1; in the case of diseases or disorders associated with or involving ephrin receptor signaling and/or regulation thereof in the brain, neuron, and/or CNS and/or diseases or disorders thereof: PRKCE; ITGAM; ROCK1; ITGA5; CXCR4; IRAK1; PRKAA2; EIF2AK2; RACI; RAPlA; GRK6; ROCK2; MAPK1; PGF; RAC2; PTPN11; GNAS; PLK1; AKT2; DOK1; CDK8; CREB1; PTK2; CFL1; GNAQ; MAP3K14; CXCL12; MAPK8; GNB2L1; ABL1; MAPK3; ITGA1; KRAS; RHOA; PRKCD; PRKAA1; MAPK9; SRC; CDK2; PIM1; ITGB7; PXN; RAF1; FYN; DYRKIA; ITGB1; MAP2K2; PAK4; AKT1; JAK2; STAT3; ADAM10; MAP2K1; PAK3; ITGB3; CDC42; VEGFA; ITGA2; EPHA8; TTK; CSNK1A1; CRKL; BRAF; PTPN13; ATF4; AKT3; SGK; in the case of diseases or disorders associated with or involving B cell receptor signaling and/or regulation thereof in the brain, neuron, and/or CNS and/or diseases or disorders thereof: RAC1; PTEN; LYN; ELK1; MAPK1; RAC2; PTPN11; AKT2; IKBKB; PIK3CA; CREB1; SYK; NFKB2; CAMK2A; MAP3K14; PIK3CB; PIK3C3; MAPK8; BCL2L1; ABL1; MAPK3; ETS1; KRAS; MAPK13; RELA; PTPN6; MAPK9; EGRI; PIK3C2A; BTK; MAPK14; RAF1; IKBKG; RELB; MAP3K7; MAP2K2; AKT1; PIK3R1; CHUK; MAP2K1; NFKB1; CDC42; GSK3A; FRAP1; BCL6; BCL10; JUN; GSK3B; ATF4; AKT3; VAV3; RPS6KB1.

Additional non-limiting examples of disease-associated genes and polynucleotides and disease specific information is available from McKusick-Nathans Institute of Genetic Medicine, Johns Hopkins University (Baltimore, Md.) and National Center for Biotechnology Information, National Library of Medicine (Bethesda, Md.), available on the World Wide Web. Any of these can be appropriate to be treated by one or more of the methods described herein. In some embodiments, the disease is a muscle disease or disorder, neuro-muscular disease or disorder, or a cardiomyopathy. In some embodiments, the disease or disorder selected from any one or more of the following: (a) an auto immune disease; (b) a cancer; (c) a muscular dystrophy; (d) a neuro-muscular disease; (e) a sugar or glycogen storage disease; (f) an expanded repeat disease; (g) a dominant negative disease; (h) a cardiomyopathy; (i) a viral disease; (j) a progeroid disease; or (k) any combination thereof.

In some embodiments, the expanded repeat disease is Huntington's disease, a Myotonic Dystrophy, or Facioscapulohumeral muscular dystrophy (FSHD). In some embodiments, the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD. In some embodiments, the myotonic dystrophy is Type 1 or Type 2. In some embodiments, the cardiomyopathy is dilated cardiomyopathy, hypertrophic cardiomyopathy, DMD-associated cardiomyopathy, or Dannon disease. In some embodiments, the sugar or glycogen storage disease is a MPS type III disease or Pompe disease. In some embodiments, the MPS type III disease, is MPS Type IIIA, IIIB, IIIC, or IIID. In some embodiments, the neuro-muscular disease is Charcot-Marie-Tooth disease or Friedreich's Ataxia.

More specifically, Mutations in these genes and pathways can result in production of improper proteins or proteins in improper amounts which affect function. Further examples of genes, diseases and proteins are hereby incorporated by reference from U.S. Provisional application 61/736,527 filed Dec. 12, 2012. Such genes, proteins and pathways may be the target polynucleotide of a CRISPR complex or other method of gene modification of the present invention. Examples of disease-associated and/or cell function-associated genes and polynucleotides are listed in Tables 2 and 3. Additional examples are discussed elsewhere herein.

TABLE 2
Exemplary Genetic and Other Diseases and Associated Genes

| | Primary | Additional | |
|---|---|---|---|
| | Tissues or | Tissues/ | |
| | System | Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune | | KIR3DL1, NKAT3, NKB1, AMB11, |
| | System | | KIR3DS1, IFNG, CXCL12, SDF1 |
| Albinism (including | Skin, hair, | | TYR, OCA2, TYRP1, and SLC45A2, |
| oculocutaneous albinism (types | eyes, | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; ALS7; |
| | | | STEX; FUS; TARDBP; VEGF |
| | | | (VEGF-a; VEGF-b; VEGF-c); DPP6; |
| | | | NEFH, PTGS1, SLC1A2, |
| | | | TNFRSF10B, PRPH, HSP90AA1, |
| | | | CRIA2, IFNG, AMPA2 S100B, FGF2, |
| | | | AOX1, CS, TXN, RAPHJ1, MAP3K5, |
| | | | NBEAL1, GPX1, ICA1L, RAC1, |
| | | | MAPT, ITPR2, ALS2CR4, GLS, |
| | | | ALS2CR8, CNTFR, ALS2CR11, |
| | | | FOLH1, FAM117B, P4HB, CNTF, |
| | | | SQSTM1, STRADB, NAIP, NLR, |
| | | | YWHAQ, SLC33A1, TRAK2, SCA1, |
| | | | NIF3L1, NIF3, PARD3B, COX8A, |
| | | | CDK15, HECW1, HECT, C2, WW 15, |
| | | | NOS1, MET, SOD2, HSPB1, NEFL, |
| | | | CTSB, ANG, HSPA8, RNase A, |
| | | | VAPB, VAMP, SNCA, alpha HGF, |
| | | | CAT, ACTB, NEFM, TH, BCL2, |
| | | | FAS, CASP3, CLU, SMN1, G6PD, |
| | | | BAX, HSF1, RNF19A, JUN, |
| | | | ALS2CR12, HSPA5, MAPK14, |
| | | | APEX1, TXNRD1, NOS2, TIMP1, |
| | | | CASP9, XIAP, GLG1, EPO, VEGFA, |
| | | | ELN, GDNF, NFE2L2, SLC6A3, |
| | | | HSPA4, APOE, PSMB8, DCTN2, |
| | | | TIMP3, KIFAP3, SLC1A1, SMN2, |
| | | | CCNC, STUB1, ALS2, PRDX6, SYP, |
| | | | CABIN1, CASP1, GART, CDK5, |
| | | | ATXN3, RTN4, C1QB, VEGFC, |
| | | | HTT, PARK7, XDH, GFAP, MAP2, |
| | | | CYCS, FCGR3B, CCS, UBL5, |
| | | | MMP9m SLC18A3, TRPM7, HSPB2, |
| | | | AKT1, DEERL1, CCL2, NGRN, GSR, |
| | | | TPPP3, APAF1, BTBD10, GLUD1, |
| | | | CXCR4, S:C1A3, FLT1, PON1, AR, |
| | | | LIF, ERBB3, :GA:S1, CD44, TP53, |
| | | | TLR3, GRIA1, GAPDH, AMPA, |
| | | | GRIK1, DES, CHAT, FLT4, |
| | | | CHMP2B, BAG1, CHRNA4, GSS, |
| | | | BAK1, KDR, GSTP1, OGG1, IL6 |
| Alzheimer's Disease | Brain | | E1; CHIP; UCH; UBB; Tau; LRP; |
| | | | PICALM; CLU; PS1; SORL1; CR1; |
| | | | VLDLR; UBA1; UBA3; CHIP28; |
| | | | AQP1; UCHL1; UCHL3; APP, AAA, |
| | | | CVAP, AD1, APOE, AD2, DCP1, |
| | | | ACE1, MPO, PACIP1, PAXIP1L, |
| | | | PTIP, A2M, BLMH, BMH, PSEN1, |
| | | | AD3, ALAS2, ABCA1, BIN1, BDNF, |
| | | | BTNL8, C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, ILIRN, IL- |
| | | | IRA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMR1, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD)— | | | |
| (includes diseases such as von | | | |
| Hippel-Lindau disease and | | | |
| tuberous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2 ) | | | |
| Bleeding Disorders | blood | | TBXA2R, P2RX1, P2X1 |
| Blue Cone Monochromacy | eye | | OPNILW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | |
| | | | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PONI, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAVI, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RACI, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADHIC, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADHIB, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNAIC, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MATIA, OPRL1, IMPA1, CLCN2, |
| | | | DLD, PSMA6, PSMB8, CHI3L1, |
| | | | ALDH1B1, PARP2,STAR, LBP, |
| | | | ABCC6, RGS2, EFNB2, GJB6, |
| | | | APOA2, AMPD1, DYSF, |
| | | | FDFT1,EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SATI, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2,TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP, ANGPLT3 |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQPO, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT2O), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chediak-Higashi Syndrome | Immune system | Skin, hair, eyes, | LYST |
| | | neurons | |
| Choroideremia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC |
| adenomatous polyposis (FAP) | | | HNPCC: MSH2, MLH1, PMS2, SH6, |
| and hereditary nonpolyposis | | | PMS1 |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUAIA, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POCIB, RAX2, |
| | | | RDH5, RPGRIP1, TTLL5, RetCG1, |
| | | | GUCY2E |
| Congenital Stationary Night | eye | | CABP4, CACNAIF, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44 |
| Dent Disease (Types 1 and 2) | Kidney | | Type 1: CLCN5, Type 2: ORCL |
| Dentatorubral-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; GRIA2; |
| | | | Grm5; Grin1; Htr1b; Grin2a; Drd3; |
| | | | Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various— | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholesterolemia | Cardiovascular | | APOB, LDLR, PCSK9 |
| | system | | |
| Familial Mediterranean Fever | Various— | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various—blood | | FANCA, FACA, FAI, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various—where | GALT, GALK1, and GALE |
| | metabolism | galactose | |
| | disorder | accumulates— | |
| | | liver, brain, eyes | |
| Gastrointestinal Epithelial | | | CISH |
| Cancer, GI cancer | | | |
| Gaucher Disease (Types 1, 2, and | Fat metabolism | Various—liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulosclerosis | kidney | | CC chemokine ligand 2 |
| Glycogen Storage Diseases | Metabolism | | SLC2A2, GLUT2, G6PC, G6PT, |
| Types I-VI—See also Cori's | Diseases | | G6PT1, GAA, LAMP2, LAMPB, |
| Disease, Pompe Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup disease | Malabsorption | Various—brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various— | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB, FIX |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXINI, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various— | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various—liver, | DDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various—liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various—liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g., galactosemia), Amino |
| organic acidemias), fatty acid | | | acid Metabolism disorders (e.g., |
| oxidation defects, amino | | | phenylketonuria), Fatty acid |
| acidopathies, carbohydrate | | | metabolism (e.g., MCAD deficiency), |
| disorders, mitochondrial | | | Urea Cycle disorders (e.g., |
| disorders | | | Citrullinemia), Organic acidemias |
| | | | (e.g., Maple Syrup Urine disease), |
| | | | Mitochondrial disorders (e.g., |
| | | | MELAS), peroxisomal disorders (e.g., |
| | | | Zellweger syndrome) |
| Inflammation | Various | | IL-10; IL-1 (IL-11a; IL-1b); IL-13; IL- |
| | | | 17 (IL-17a (CTLA8); IL-17b; IL-17c; |
| | | | IL-17d; IL-17f); II-23; Cx3cr1; |
| | | | ptpn22; TNFa; NOD2/CARD15 for |
| | | | IBD; IL-6; IL-12 (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g., Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-B type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPLI, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various—joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSCIL1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHMI, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HTRA1, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinese | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease | | Hurler disease, Sanfilippo syndrome, |
| C, I S, IVA and B, IX, VII, and | - affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; Notch1; Notch2; |
| | | | Notch3; Notch4; AKT; AKT2; AKT3; |
| | | | HIF; HIF1a; HIF3a; Met; HRG; Bcl2; |
| | | | PPAR alpha; PPAR gamma; WT1 |
| | | | (Wilms Tumor); FGF Receptor Family |
| | | | members (5 members: 1, 2, 3, 4, 5); |
| | | | CDKN2a; APC; RB (retinoblastoma); |
| | | | MEN1; VHL; BRCA1; BRCA2; AR |
| | | | (Androgen Receptor); TSG101; IGF; |
| | | | IGF Receptor; Igf1 (4 variants); Igf2 (3 |
| | | | variants); Igf 1 Receptor; Igf 2 |
| | | | Receptor; Bax; Bcl2; caspases family |
| | | | (9 members: 1, 2, 3, 4, 6, 7, 8, 9, 12); |
| | | | Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various—where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe Disease | Glycogen | Various—heart, | GAA |
| | storage disease | liver, spleen | |
| Porphyria (actually refers to a | Various— | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g., type | Various—eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various—liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); Complexin1 (Cplx1); |
| | | | Tph1 Tryptophan hydroxylase; Tph2 |
| | | | Tryptophan hydroxylase 2; Neurexin |
| | | | 1; GSK3; GSK3a; GSK3b; 5-HTT |
| | | | (Slc6a4); COMT; DRD (Drd1a); |
| | | | SLC6A3; DAOA; DTNBP1; Dao |
| | | | (Dao1); TCF4; COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1,PAWR, NOTCJ2, M6PR, |
| | | | CYP46A1, CSNK1D, MAPK14, |
| | | | PRG2, PRKCA, L1 CAM, CD40, |
| | | | NR112, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOFIA, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLREIC, ARTEMIS, |
| Immunodeficiency (SCID) and | | | SCIDA, RAG1, RAG2, ADA, PTPRC, |
| SCID-X1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 and |
| | | | −130 of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome;22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; FXN/X25 |
| (generally) | | | ATX3; ATXN1; ATXN2; DMPK; |
| | | | Atrophin-1 and Atn1 (DRPLA Dx); |
| | | | CBP (Creb-BP—global instability); |
| | | | VLDLR; Atxn7; Atxn10; FEN1, |
| | | | TNRC6A, PABPN1, JPH3, MED 15, |
| | | | ATXN1, ATXN3, TBP, CACNA1A, |
| | | | ATXN80S, PPP2R2B, ATXN7, |
| | | | TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various— | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various— | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardio-facial | | | |
| syndrome) | | | |
| Von Gierke's Disease (Glycogen | Glycogen | Various—liver, | G6PC and SLC37A4 |
| Storage Disease type I) | Storage disease | kidney | |
| Von Hippel-Lindau Syndrome | Various—cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various— | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

TABLE 3
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKNIB; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRKIA; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STATI; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKNIA; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; |
| Signaling | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor | PRKCE; ITGAM; ROCK1; ITGA5; CXCR4; IRAK1; |
| Signaling | PRKAA2; EIF2AK2; RAC1; RAPIA; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRKIA; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; ILIR1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKNIA; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon | HSPB1; EP300; FASN; TGM2; RXRA; MAPK1; NQO1; |
| Receptor Signaling | NCOR2; SP1; ARNT; CDKN1B; FOS; CHEK1; |
| | SMARCA4; NFKB2; MAPK8; ALDHIA1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKNIA; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGCIA; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| Signaling | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNKIA1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell | PRKCE; RAC1; PRKCZ; MAPK1; RAC2; PTPN11; |
| Signaling | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKNIB; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKNIA; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKNIA; MAP2K2; JAK1; |
| | AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; |
| Nicotinamide Metabolism | PLK1; AKT2; CDK8; MAPK8; MAPK3; PRKCD; PRKAA1; |
| | PBEF1; MAPK9; CDK2; PIM1; DYRKIA; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGCIA; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; ILIR1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKNIA; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor | IRAK1; EIF2AK2; MYD88; TRAF6; PPARA; ELK1; |
| Signaling | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; ILIR1; |
| | SRF; STAT1 |
| Neurotrophin/TRK | NTRK2; MAPK1; PTPN11; PIK3CA; CREB1; FOS; |
| Signaling | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAPIA; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated | IRAK1; MYD88; TRAF6; PPARA; RXRA; ABCA1; |
| Inhibition of RXR | MAPK8; ALDH1A1; GSTP1; MAPK9; ABCB1; TRAF2; |
| Function | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling | KDR; FLT1; PGF; AKT2; PIK3CA; PIK3CB; PIK3C3; |
| in the Cardiovascular | CAV1; PRKCD; NOS3; PIK3C2A; AKT1; PIK3R1; |
| System | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| CAMP-mediated | RAP1A; MAPK1; GNAS; CREB1; CAMK2A; MAPK3; |
| Signaling | SRC; RAF1; MAP2K2; STAT3; MAP2K1; BRAF; ATF4 |
| Mitochondrial | SOD2; MAPK8; CASP8; MAPK10; MAPK9; CASP9; |
| Dysfunction Notch | PARK7; PSEN1; PARK2; APP; CASP3 |
| Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress | EIF2AK3; CASP3 |
| Pathway Pyrimidine | NME2; AICDA; RRM2; EIF2AK4; ENTPD1; RRM2B; |
| Metabolism | NT5E; POLD1; NME1 |
| Parkinson's Signaling | UCHL1; MAPK8; MAPK13; MAPK14; CASP9; PARK7; |
| | PARK2; CASP3 |
| Cardiac & Beta | GNAS; GNAQ; PPP2R1A; GNB2L1; PPP2CA; PPP1CC; |
| Adrenergic Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Amino sugars Metabolism | NQO1; HK2; GCK; HK1 |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPPICC; PPP2R5C |
| Signaling | |
| Glutathione Metabolism | IDH2; GSTP1; ANPEP; IDH1 |
| Glycerolipid Metabolism | ALDHIA1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDHIA1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate Pathway | GPI |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

Thus, also described herein are methods of inducing one or more mutations in a eukaryotic or prokaryotic cell (in vitro, i.e., in an isolated eukaryotic cell) as herein discussed comprising delivering to cell a vector as described herein. The mutation(s) can include the introduction, deletion, or substitution of one or more nucleotides at a target sequence of cell(s). In some embodiments, the mutations can include the introduction, deletion, or substitution of 1-75 nucleotides at each target sequence of said cell(s). The mutations can include the introduction, deletion, or substitution of 1, 5, 10, 11, 12, 13, 14, 15, 16, 17, 18, 19, 20, 21, 22, 23. 24. 25, 26. 27. 28. 29. 30. 35, 40. 45, 50, or 75 nucleotides at each target sequence. The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The mutations can include the introduction, deletion, or substitution of 20, 21, 22, 23, 24, 25, 26, 27, 28, 29, 30, 35, 40, 45, 50, or 75 nucleotides at each target sequence of said cell(s). The mutations can include the introduction, deletion, or substitution of 40, 45, 50, 75, 100, 200, 300, 400 or 500 nucleotides at each target sequence of said cell(s). The mutations can include the introduction, deletion, or substitution of 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In some embodiments, the modifications can include the introduction, deletion, or substitution of nucleotides at each target sequence of said cell(s) via nucleic acid components (e.g., guide(s) RNA(s) or sgRNA(s)), such as those mediated by a CRISPR-Cas system.

In some embodiments, the modifications can include the introduction, deletion, or substitution of nucleotides at a target or random sequence of said cell(s) via a non CRISPR-Cas system or technique. Such techniques are discussed elsewhere herein, such as where engineered cells and methods of generating the engineered cells and organisms are discussed.

For minimization of toxicity and off-target effect when using a CRISPR-Cas system, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9-like with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, a tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to a guide sequence.

In one embodiment, the invention provides a method of modifying a target polynucleotide in a eukaryotic cell. In some embodiments, the method includes delivering an engineered targeting moiety, polypeptide, polynucleotide, vector, vector system, particle, viral (e.g., AAV) particle, cell, or any combination thereof described herein having a genetic modifying agent (including, but not limited to, a CRISPR-Cas system or system component) as a cargo molecule to a subject and/or cell. The CRISPR-Cas system molecule(s) delivered can complex to bind to the target polynucleotide, e.g., to effect cleavage of said target polynucleotide, thereby modifying the target polynucleotide, wherein the CRISPR complex comprises a CRISPR enzyme complexed with a guide sequence hybridized to a target sequence within said target polynucleotide, wherein said guide sequence can be linked to a tracr mate sequence which in turn hybridizes to a tracr sequence. In some embodiments, said cleavage comprises cleaving one or two strands at the location of the target sequence by said CRISPR enzyme. In some embodiments, said cleavage results in decreased transcription of a target gene. In some embodiments, the method further comprises repairing said cleaved target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expressed from a gene comprising the target sequence. In some embodiments, the method further comprises delivering one or more vectors to said eukaryotic cell, wherein one or more vectors comprise the CRISPR enzyme and one or more vectors drive expression of one or more of: the guide sequence linked to the tracr mate sequence, and the tracr sequence. In some embodiments, said CRISPR enzyme drive expression of one or more of: the guide sequence linked to the tracr mate sequence, and the tracr sequence. In some embodiments such CRISPR enzyme are delivered to the eukaryotic cell in a subject. In some embodiments, said modifying takes place in said eukaryotic cell in a cell culture. In some embodiments, the method further comprises isolating said eukaryotic cell from a subject prior to said modifying. In some embodiments, the method further comprises returning said eukaryotic cell and/or cells derived therefrom to said subject. In some embodiments, the isolated cells can be returned to the subject after delivery of one or more engineered targeting moieties, polypeptides, polynucleotides, vectors, vector systems, particles, viral (e.g., AAV) particles, cells, or any combination thereof described herein to the isolated cell. In some embodiments, the isolated cells can be returned to the subject after delivering one or more molecules of the engineered delivery system described herein to the isolated cell, thus making the isolated cells engineered cells as previously described.

### Screening and Cell Selection

The targeting moieties, polypeptides, polynucleotides, vectors, vector systems, particles, viral (e.g., AAV) particles, cells, or any combination thereof described herein described herein can be used in a screening assay and/or cell selection assay. The engineered targeting moieties, polypeptides, polynucleotides, vectors, vector systems, particles, viral (e.g., AAV) particles, cells, or any combination thereof described herein can be delivered to a subject and/or cell. In some embodiments, the cell is a eukaryotic cell. The cell can be in vitro, ex vivo, in situ, or in vivo. The targeting moieties, polypeptides, polynucleotides, vectors, vector systems, particles, viral (e.g., AAV) particles, cells, or any combination thereof described herein can introduce an exogenous molecule or compound, such as a cargo, to subject or cell to which they are delivered. The presence of an exogenous molecule or compound can be detected which can allow for identification of a cell and/or attribute thereof. In some embodiments, the delivered molecules or particles can impart a gene or other nucleotide modification (e.g., mutations, gene or polynucleotide insertion and/or deletion, etc.). In some embodiments the nucleotide modification can be detected in a cell by sequencing. In some embodiments, the nucleotide modification can result in a physiological and/or biological modification to the cell that results in a detectable phenotypic change in the cell, which can allow for detection, identification, and/or selection of the cell. In some embodiments, the phenotypic change can be cell death, such as embodiments where binding of a CRISPR complex to a target polynucleotide results in cell death. Embodiments of the invention allow for selection of specific cells without requiring a selection marker or a two-step process that may include a counter-selection system. The cell(s) may be prokaryotic or eukaryotic cells.

In one embodiment the invention provides for a method of selecting one or more cell(s) by introducing one or more mutations in a gene in the one or more cell (s), the method comprising: introducing one or more vectors, which can include one or more engineered delivery system molecules or vectors described elsewhere herein, into the cell (s), wherein the one or more vectors can include a CRISPR enzyme and/or drive expression of one or more of: a guide sequence linked to a tracr mate sequence, a tracr sequence, and an editing template; or other polynucleotide to be inserted into the cell and/or genome thereof, wherein, for example that which is being expressed is within and expressed in vivo by the CRISPR enzyme and/or the editing template, when included, comprises the one or more mutations that abolish CRISPR enzyme cleavage; allowing homologous recombination of the editing template with the target polynucleotide in the cell(s) to be selected; allowing a CRISPR complex to bind to a target polynucleotide to effect cleavage of the target polynucleotide within said gene, wherein the CRISPR complex comprises the CRISPR enzyme complexed with (1) the guide sequence that is hybridized to the target sequence within the target polynucleotide, and (2) the tracr mate sequence that is hybridized to the tracr sequence, wherein binding of the CRISPR complex to the target polynucleotide induces cell death, thereby allowing one or more cell(s) in which one or more mutations have been introduced to be selected. In a preferred embodiment, the CRISPR enzyme is a Cas protein. In another embodiment of the invention the cell to be selected may be a eukaryotic cell.

The screening methods involving the engineered targeting moieties, polypeptides, polynucleotides, vectors, vector systems, particles, viral (e.g., AAV) particles, cells, or any combination thereof described herein, including but not limited to those that deliver one more CRISPR-Cas system molecules to cell, can be used in detection methods such as fluorescence in situ hybridization (FISH). In some embodiments, one or more components of an engineered CRISPR-Cas system that includes a catalytically inactive Cas protein, can be delivered by engineered targeting moieties, polypeptides, polynucleotides, vectors, vector systems, particles, viral (e.g. AAV) particles, cells, or any combination thereof described herein to a cell and used in a FISH method. The CRISPR-Cas system can include an inactivated Cas protein (dCas) (e.g. a dCas9), which lacks the ability to produce DNA double-strand breaks may be fused with a marker, such as fluorescent protein, such as the enhanced green fluorescent protein (eEGFP) and co-expressed with small guide RNAs to target pericentric, centric and teleomeric repeats in vivo. The dCas system can be used to visualize both repetitive sequences and individual genes in the human genome. Such new applications of labelled dCas, dCas CRISPR-Cas systems, engineered targeting moieties, polypeptides, polynucleotides, vectors, vector systems, particles, viral (e.g. AAV) particles, cells, or any combination thereof described herein can be used in imaging cells and studying the functional nuclear architecture, especially in cases with a small nucleus volume or complex 3-D structures. (Chen B, Gilbert L A, Cimini B A, Schnitzbauer J, Zhang W, Li G W, Park J, Blackburn E H, Weissman J S, Qi L S, Huang B. 2013. Dynamic imaging of genomic loci in living human cells by an optimized CRISPR/Cas system. Cell 155(7):1479-91. doi: 10.1016/j.cell.2013.12.001., the teachings of which can be applied and/or adapted to the CRISPR systems described herein. A similar approach involving a polynucleotide fused to a marker (e.g. a fluorescent marker) can be delivered to a cell via an engineered targeting moieties, polypeptides, polynucleotides, vectors, vector systems, particles, viral (e.g. AAV) particles, cells, or any combination thereof described herein and integrated into the genome of the cell and/or otherwise interact with a region of the genome of a cell for FISH analysis.

Similar approaches for studying other cell organelles and other cell structures can be accomplished by delivering to the cell (e.g., via an engineered targeting moieties, polypeptides, polynucleotides, vectors, vector systems, particles, viral (e.g. AAV) particles, cells, or any combination thereof described herein) one or more molecules fused to a marker (such as a fluorescent marker), wherein the molecules fused to the marker are capable of targeting one or more cell structures. By analyzing the presence of the markers, one can identify and/or image specific cell structures.

In some embodiments, the engineered targeting moieties, polypeptides, polynucleotides, vectors, vector systems, particles, viral (e.g., AAV) particles, cells, or any combination thereof described herein can be used in a screening assay inside or outside of a cell. In some embodiments, the screening assay can include delivering a CRISPR-Cas cargo molecule(s) via engineered targeting moieties, polypeptides, polynucleotides, vectors, vector systems, particles, viral (e.g. AAV) particles, cells, or any combination thereof described herein.

Use of the present system in screening is also provided by the present invention, e.g., gain of function screens. Cells which are artificially forced to overexpress a gene are able to down regulate the gene over time (re-establishing equilibrium) e.g., by negative feedback loops. By the time the screen starts the unregulated gene might be reduced again. Other screening assays are discussed elsewhere herein.

In an embodiment, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results.

In an embodiment, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results; and wherein the cell product is altered compared to the cell not contacted with the delivery system, for example altered from that which would have been wild type of the cell but for the contacting. In an embodiment, the cell product is non-human or animal. In some embodiments, the cell product is human.

In some embodiments, a host cell is transiently or non-transiently transfected with one or more vectors described herein. In some embodiments, a cell is transfected as it naturally occurs in a subject optionally to be reintroduced therein. In some embodiments, a cell that is transfected is taken from a subject. In some embodiments, the cell obtained from or is derived from cells taken from a subject, such as a cell line. Delivery mechanisms and techniques of the targeting moieties, polypeptides, polynucleotides, vectors, vector systems, particles, viral (e.g., AAV) particles, cells, or any combination thereof described herein.

In some embodiments it is envisaged to introduce one or more of the engineered targeting moieties, polypeptides, polynucleotides, vectors, vector systems, particles, viral (e.g., AAV) particles, cells, or any combination thereof described herein directly to the host cell. For instance, the engineered AAV capsid system molecule(s) can be delivered together with one or more cargo molecules to be packaged into an engineered AAV particle.

In some embodiments, the invention provides a method of expressing an engineered delivery molecule and cargo molecule to be packaged in an engineered viral (e.g. AAV) particle in a cell that can include the step of introducing the vector according any of the vector delivery systems disclosed herein.

The invention is further described in the following examples, which do not limit the scope of the invention described in the claims.

Further embodiments are illustrated in the following Examples which are given for illustrative purposes only and are not intended to limit the scope of the invention.

### EXAMPLES

### Example 1—mRNA Based Detection Methods are More Stringent for Selection of AAV Variants

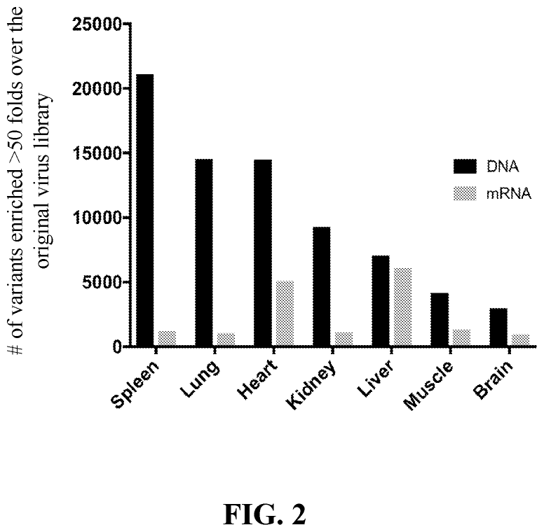

FIG. 1 demonstrates the adeno-associated virus (AAV) transduction mechanism, which results in production of mRNA. As is demonstrated in FIG. 1, functional transduction of a cell by an AAV particle can result in the production of an mRNA strand. Non-functional transduction would not produce such a product despite the viral genome being detectable using a DNA-based assay. Thus, mRNA-based detection assays to detect transduction by e.g., an AAV can be more stringent and provide feedback as to the functionality of a virus particle that is able to functionally transduce a cell. FIG. 2 shows a graph that can demonstrate that mRNA-based selection of AAV variants can be more stringent than DNA-based selection. The virus library was expressed under the control of a CMV promoter.

### Example 2—mRNA Based Detection Methods can be Used to Detect AAV Capsid Variants from a Capsid Variant Library

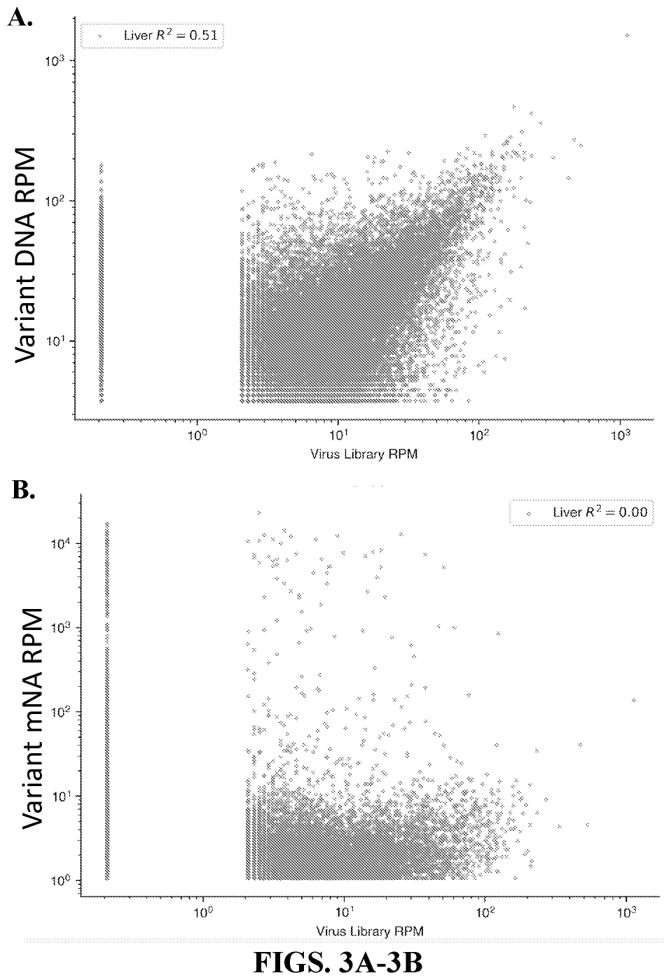

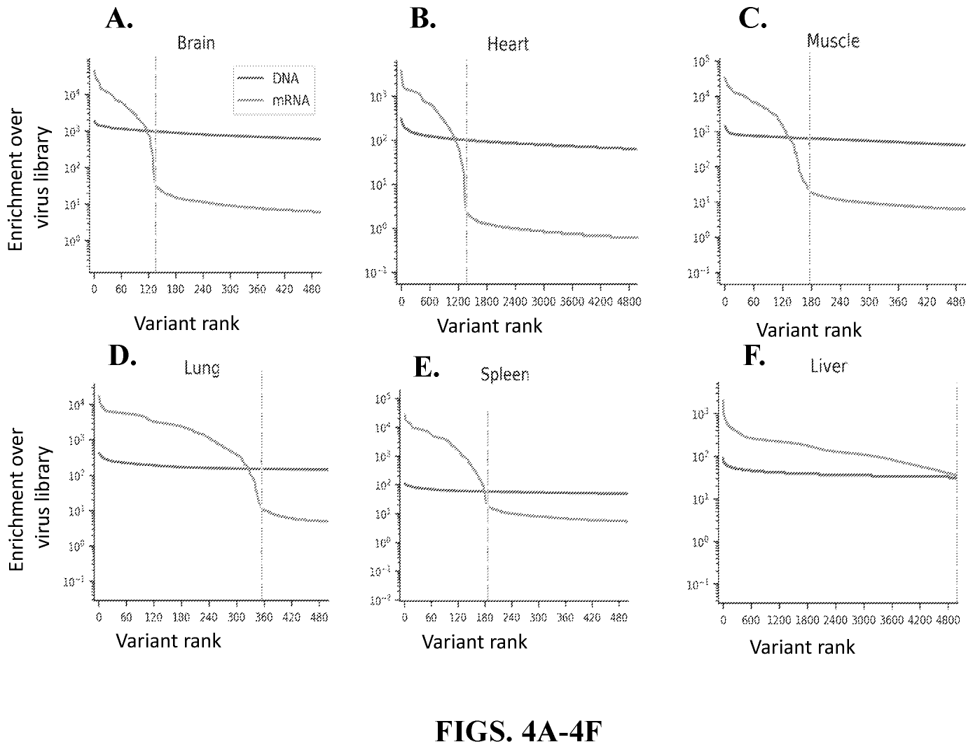

FIGS. 3A-3B show graphs that can demonstrate a correlation between the virus library and vector genome DNA (FIG. 3A) and mRNA (FIG. 3B) in the liver. FIGS. 4A-4F show graphs that can demonstrate capsid variants expressed at the mRNA level identified in different tissues.

### Example 3—Capsid mRNA Expression can be Driven by Tissue Specific Promoters

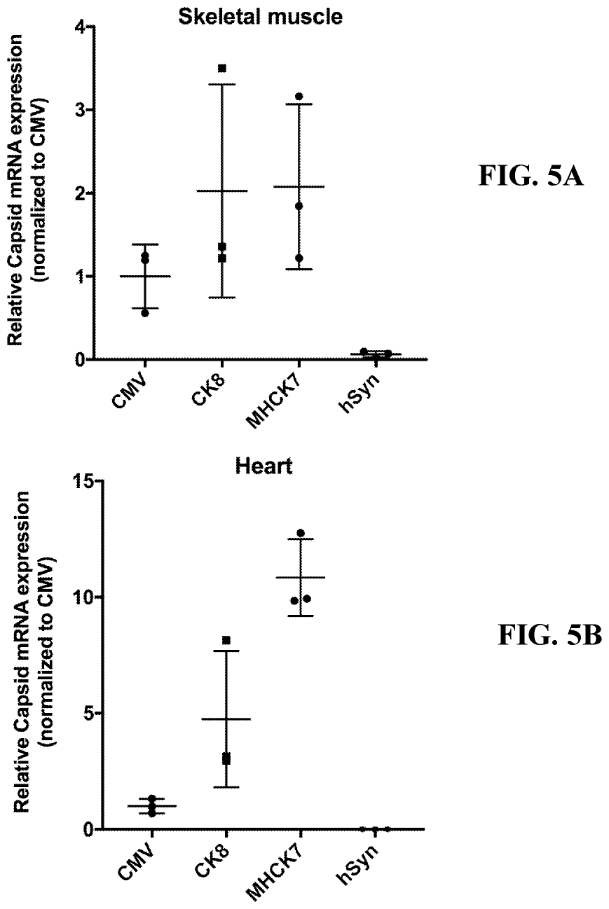

FIGS. 5A-5C show graphs that can demonstrate capsid mRNA expression in different tissues under the control of cell-type specific promoters (as noted on x-axis). CMV was included as an exemplary constitutive promoter. CK8 is a muscle-specific promoter. MHCK7 is a muscle-specific promoter. hSyn is a neuron specific promoter.

### Example 4—Capsid Variant Library Generation, Variant Screening, and Variant Dentification

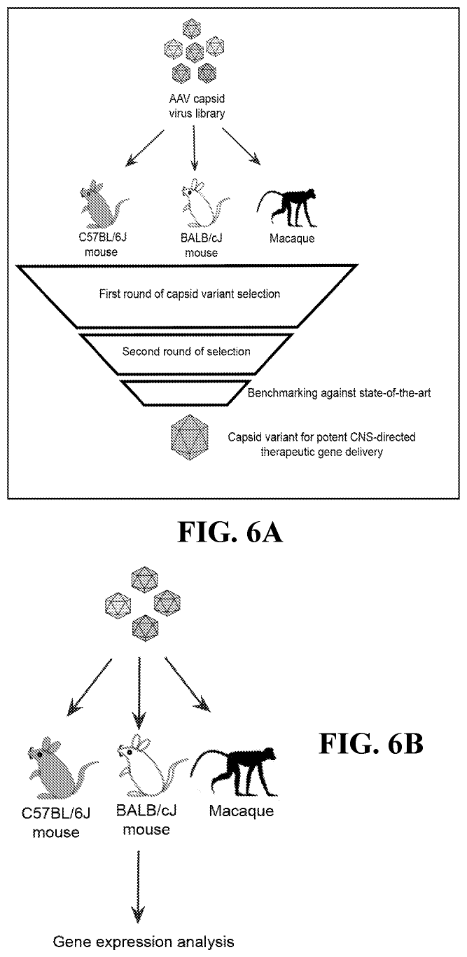

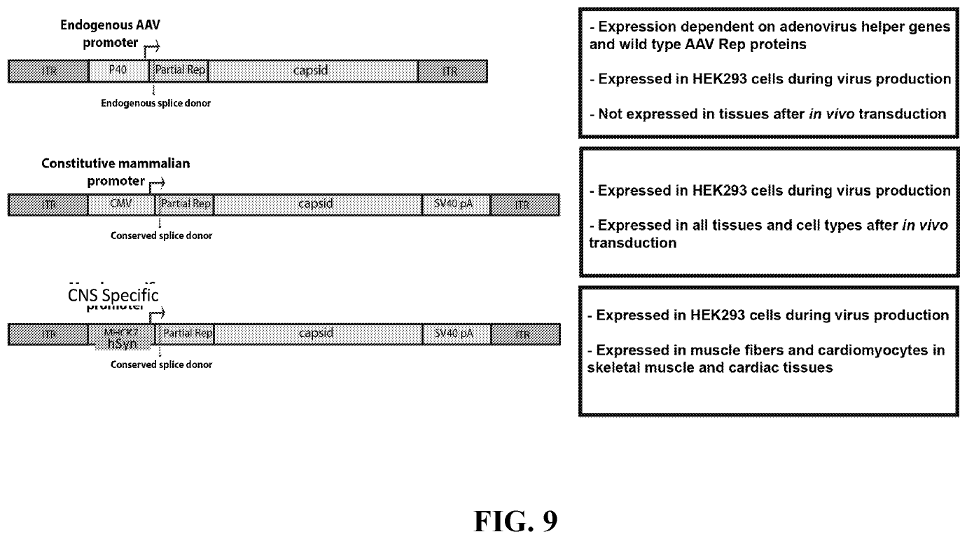

Generally, an AAV capsid library can be generated by expressing engineered capsid vectors each containing an engineered AAV capsid polynucleotide previously described in an appropriate AAV producer cell line. See e.g., FIG. 8. This can generate an AAV capsid library that can contain one more desired cell-specific engineered AAV capsid variant. FIG. 7 shows a schematic demonstrating embodiments of generating an AAV capsid variant library, particularly insertion of a random n-mer (n=3-15 amino acids) into a wild-type AAV, e.g., AAV9. In this example, random 7-mers were inserted between aa588-589 of variable region VIII of AAV9 viral protein and used to form the viral genome containing vectors with one variant per vector. As shown in FIG. 8, the capsid variant vector library was used to generate AAV particles where each capsid variant encapsulated its coding sequence as the vector genome. FIG. 9 shows vector maps of representative AAV capsid plasmid library vectors (see e.g., FIG. 8) that can be used in an AAV vector system to generate an AAV capsid variant library. The library can be generated with the capsid variant polynucleotide under the control of a tissue specific promoter or constitutive promoter. The library was also made with capsid variant polynucleotide that included a polyadenylation signal.

As shown in FIG. 6A the AAV capsid library can be administered to various non-human animals for a first round of mRNA-based selection. As shown in FIG. 1, the transduction process by AAVs and related vectors can result in the production of an mRNA molecule that is reflective of the genome of the virus that transduced the cell. As is at least demonstrated in the Examples herein, mRNA based selection can be more specific and effective to determine a virus particle capable of functionally transducing a cell because it is based on the functional product produced as opposed to just detecting the presence of a virus particle in the cell by measuring the presence of viral DNA.

As is further shown in FIG. 6A, fter first-round administration, one or more engineered AAV virus particles having a desired capsid variant can then be used to form a filtered AAV capsid library. Desirable AAV virus particles can be identified by measuring the mRNA expression of the capsid variants and determining which variants are highly expressed in the desired cell type(s) as compared to non-desired cells type(s). Those that are highly expressed in the desired cell, tissue, and/or organ type are the desired AAV capsid variant particles. In some embodiments, the AAV capsid variant encoding polynucleotide is under control of a tissue-specific promoter that has selective activity in the desired cell, tissue, or organ.

The engineered AAV capsid variant particles identified from the first round can then be administered to various non-human animals. In some embodiments, the animals used in the second round of selection and identification are not the same as those animals used for first round selection and identification. Similar to round 1, after administration the top expressing variants in the desired cell, tissue, and/or organ type(s) can be identified by measuring viral mRNA expression in the cells. The top variants identified after round two can then be optionally barcoded and optionally pooled. In some embodiments, top variants from the second round can then be administered to a non-human primate to identify the top cell-specific variant(s), particularly if the end use for the top variant is in humans. Administration at each round can be systemic. As further shown in FIG. 6B after the second round of selection, a third round of selection, which can optionally include benchmarking against known, control, and/or standard (e.g., benchmark) variants can be performed.

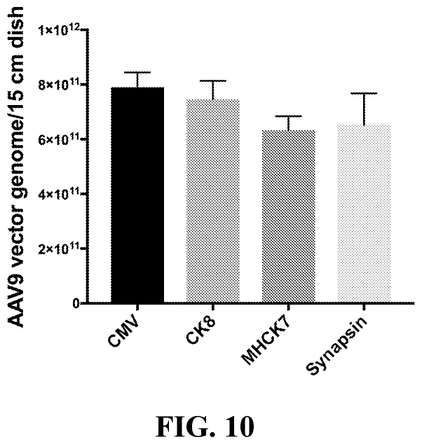

FIG. 10 shows a graph that can demonstrate the viral titer (calculated as AAV9 vector genome/15 cm dish) produced by libraries generated using different promoters. As demonstrated in FIG. 10, virus titer was not affected significantly be the use of different promoters.

### Example 5—Motif Variants Having Muscle Specificity or Both Muscle and CNS Specificity

As shown in FIGS. 6A-6B shows a general schematic for selecting CNS specific capsid, which includes a benchmarking round which evaluates the performance of selected capsids against currently used capsids for, e.g., delivery to the CNS. Tables 4-11 shows the top ranking capsid variants produced in rounds of directed evolution of capsid variants for skeletal muscle specificity, cardiac muscle specificity, CNS and Cardiac muscle septicity, or CNS and skeletal muscle specificity in NHPs. As shown in the Tables below with at least respect to those variant n-mer inserts containing P-motifs the first three amino acids of the variant sequences shown are amino acids that replaced amino acids corresponding to positions 596, 597, and 598 of an AAV9 capsid polypeptide. Thus, the P-motif, for example, was inserted between amino acids at positions 598 and 599 of the AAV9 vector.

TABLE 4
Top Ranking Skeletal Muscle Specific n-mer
inserts and/or RGD Motifs

| Variant | SEQ ID NO: |
|---|---|
| TTGRGDQARL | 4 |
| TTLRGDHSQL | 5 |
| TNLRGDYQLT | 6 |
| STSRGDYVTH | 7 |
| MPARGDLVQT | 8 |
| SGVRGDRLAV | 9 |
| SNERGDQHRL | 10 |
| STLRGDYAVS | 11 |
| SNARGDIMTT | 12 |
| EASRGDLQMR | 13 |
| TYARGDLSGH | 14 |
| DSSRGDRSSV | 15 |
| SQSRGDLVPL | 16 |
| SATRGDYSIL | 17 |
| QATRGDLQHT | 18 |
| HDDRGDRRML | 19 |
| SSHRGDLLAT | 20 |
| SYQRGDQHNM | 21 |
| SHQRGDYNTL | 22 |
| MDQRGDMSVV | 23 |
| QIARGDLHVP | 24 |
| TYTRGDQHMM | 25 |
| DASRGDRTSL | 26 |
| SDRRGDLHPQ | 27 |
| SATRGDHSTL | 28 |
| AYQRGDFTSV | 29 |
| TGNRGDLQSV | 30 |
| AWRRGDLENA | 31 |
| NAARGDHGLL | 32 |
| SYSRGDHQAL | 33 |
| DGVRGDRTVV | 34 |
| SSERGDKMST | 35 |
| TSMRGDYTHV | 36 |
| SGVRGDRIII | 37 |
| ENRRGDAQLL | 38 |
| TVYRGDREQH | 39 |
| LDRRGDLLNS | 40 |
| ESVRGDLRST | 41 |
| DKYRGDQRGL | 42 |
| TDRRGDAHIL | 43 |
| SQTRGDTSLM | 44 |
| DATRGDTRML | 45 |
| SMQRGDLTHA | 46 |
| SGTRGDTLIL | 47 |
| ANTRGDHQNF | 48 |
| TSQRGDHTQL | 49 |
| SHWRGDTSSL | 50 |
| ISGRGDRQEL | 51 |
| SVLRGDIGTI | 52 |
| QDSRGDLSGK | 53 |
| KPIRGDGAEL | 54 |
| TYMRGDLQHT | 55 |
| TELRGDLSST | 56 |
| SAMRGDHAVL | 57 |
| SWVRGDASTL | 58 |
| QYARGDPHAL | 59 |
| STNRGDIVVH | 60 |
| VNIRGDLHQT | 61 |
| REYRGDSSIL | 62 |
| STLRGDGSMM | 63 |
| SSQRGDITAH | 64 |
| TEFRGDRSTV | 65 |
| TSTRGDYVTV | 66 |
| NHLRGDVSAI | 67 |
| LERRGDLVST | 68 |
| NEYRGDHSHP | 69 |
| NWQRGDTSTL | 70 |
| TDSRGDRTVL | 71 |
| YAVRGDQHTI | 72 |
| FGVRGDTRDL | 73 |
| SSARGDTGML | 74 |
| SAMRGDQMGQ | 75 |
| SHTRGDLVTT | 76 |
| VTTRGDTAIL | 77 |
| EPTRGDRHSI | 78 |
| GSQRGDRLVL | 79 |
| QHYRGDLSTT | 80 |
| METRGDRQQL | 81 |
| EALRGDKVVL | 82 |
| NAVRGDQSYV | 83 |
| SPLRGDNRAL | 84 |
| ENRRGDTHAL | 85 |
| NSQRGDLTGT | 86 |
| QYAPSQGTV | 87 |
| DNSRGDHSRL | 88 |
| NTHRGDYASI | 89 |
| HTTRGDQQPL | 90 |
| EWRRGDQTAL | 91 |
| TAVRGDQTTV | 92 |
| EYLRGDRISQ | 93 |
| SRYRGDQETI | 94 |
| ASLRGDTVTL | 95 |
| VGVRGDSHVL | 96 |
| TNSRGDHTSL | 97 |
| VDQRGDQRAL | 98 |
| ASVRGDRLDV | 99 |
| TSYRGDLSSN | 100 |
| TSDRGDSSNR | 101 |
| SYMRGDLDMR | 102 |
| TDVRGDRSMI | 103 |
| SYVRGDSSTV | 104 |
| QVYRGDRESV | 105 |
| TDRRGDAVSF | 106 |
| TNSRGDLHTT | 107 |
| EAIRGDFKTL | 108 |
| TGIRGDTAGL | 109 |
| TSSRGDVQSL | 110 |
| SSRRGDTNEL | 111 |
| SNARGDHTPL | 112 |
| SELRGDRSPY | 113 |
| VHARGDTSIM | 114 |
| SALRGDHGTL | 115 |
| QTGRGDIAGM | 116 |
| TVARGDQIRL | 117 |
| TNSRGDSVAL | 118 |
| QNARGDSAVI | 119 |
| HYLRGDTTTL | 120 |
| ANARGDLQNT | 121 |
| QNIRGDIRTV | 122 |
| AALRGDHGQM | 123 |
| TTDRGDLSAR | 124 |
| LNVRGDLVHT | 125 |
| YSTRGDHTVL | 126 |
| TNARGDLTVS | 127 |
| TNARGDYTAL | 128 |
| AEGRGDTRGS | 129 |
| TNIRGDQSSV | 130 |
| VDRRGDTMTM | 131 |
| QIGRGDVTPL | 132 |
| MQARGDLLGV | 133 |
| NTQRGDYASV | 134 |
| SDTRGDRITQ | 135 |
| SAIRGDYRDV | 136 |
| VGVRGDVHPL | 137 |
| TVLRGDAERL | 138 |
| ASTRGDLSGQ | 139 |
| DSRRGDGQNL | 140 |
| IDKRGDIQGL | 141 |
| TVTRGDLLVN | 142 |
| DSTRGDRLGL | 143 |
| AANRGDYASL | 144 |
| RDYRGDTSSL | 145 |
| SNIRGDASLH | 146 |
| NLQRGDRTDF | 147 |
| NASRGDHSSM | 148 |
| TSMRGDYTHV | 149 |
| TSRRGDLNDV | 150 |
| QSHRGDVQSL | 151 |
| STTRGDYQSV | 152 |
| YSTRGDHAML | 153 |
| TSARGDLIHT | 154 |
| ATARGDRESF | 155 |
| ASTRGDYSTM | 156 |
| SNNRGDVQRL | 157 |
| ERLRGDQHII | 158 |
| SSIRGDHATL | 159 |
| NPTRGDIATV | 160 |
| MSTRGDLSVN | 161 |
| MDRRGDITYA | 162 |
| NATRGDLMSA | 163 |
| SDHRGDRTYQ | 164 |
| NWVRGDLSTA | 165 |
| SSHRGDLSAT | 166 |
| NATRGDYREI | 167 |
| DTLRGDLQTA | 168 |
| IAVRGDLTPV | 169 |
| TLLRGDLAHS | 170 |
| MNTRGDHTVL | 171 |
| TGHRGDIAVY | 172 |
| TSNRGDQMSN | 173 |
| SATRGDHTAI | 174 |
| SSVRGDREHA | 175 |
| TNSRGDLVVH | 176 |
| ETRRGDLSSQ | 177 |
| TYARGDITLT | 178 |
| SASRGDTLHL | 179 |
| DNQRGDLSSR | 180 |
| TQSRGDLRDA | 181 |
| ENRRGDLTNA | 182 |
| TSIRGDISSV | 183 |
| ISARGDTMTL | 184 |
| SRVRGDQDHW | 185 |
| SGNRGDIASY | 186 |
| NEQRGDRYNH | 187 |
| EALRGDVRAL | 188 |
| VDRRGDYTQM | 189 |
| NNVRGDYTSV | 190 |
| QTYRGDASVL | 191 |
| SNYRGDHGSL | 192 |
| TSSRGDLREV | 193 |
| QYTRGDVSNL | 194 |
| SDLRGDVRER | 195 |
| QNARGDTTAL | 196 |
| NSLRGDRDTM | 197 |
| SNSRGDQAQV | 198 |
| EATRGDRSTL | 199 |
| VPQRGDITNL | 200 |
| DKIRGDLRSI | 201 |
| SAARGDYSIV | 202 |
| VGLRGDLIGQ | 203 |
| QAGRGDLTVI | 204 |
| SKTRGDFQEA | 205 |
| GTARGDYSML | 206 |
| ISTRGDLVTI | 207 |
| TGTRGDREYM | 208 |
| MTTRGDLSIT | 209 |
| STSRGDMTSV | 210 |
| GPARGDFTSS | 211 |
| VDRRGDLGHV | 212 |
| NSGRGDMSQL | 213 |
| SQGRGDLERY | 214 |
| VFGRGDQYTL | 215 |
| IVGRGDLHLQ | 216 |
| SYSRGDTSRL | 217 |
| MSSRGDRATI | 218 |
| MSSRGDLTRV | 219 |
| QHTRGDFGNL | 220 |
| ETRRGDLSSQ | 221 |
| TGTRGDLRAI | 222 |
| QMARGDLSTA | 223 |
| LVERGDRIMN | 224 |
| GLVRGDLNAR | 225 |
| NNPRGDLSSV | 226 |
| HEKRGDLSNS | 227 |
| SQARGDLHQT | 228 |
| FTGRGDLRTA | 229 |
| QSIRGDTGHL | 230 |
| SAGRGDMSVL | 231 |
| SERRGDEHKW | 232 |
| TTGRGDLVSL | 233 |
| LGLRGDQIGQ | 234 |
| VALRGDLTHQ | 235 |
| MIGRGDQAPL | 236 |
| FQARGDHSTL | 237 |
| NSVRGDHSSL | 238 |
| WPNRGDLQST | 239 |
| SASRGDYQTL | 240 |
| TMQRGDLHTI | 241 |
| TALRGDLAGT | 242 |
| VNARGDFSQT | 243 |
| GWIRGDRTSM | 244 |
| TFSRGDHTVL | 245 |
| SGARGDLVGV | 246 |
| NNVRGDYTSV | 247 |
| QTARGDHVVL | 248 |
| TLIRGDLTRS | 249 |
| VTTRGDRLEL | 250 |

TABLE 5
Top Ranking Skeletal Muscle Specific n-mer
inserts and/or RGD Motifs

| Variant | SEQ ID NO: |
|---|---|
| EASRGDLQMR | 251 |
| TYARGDLSGH | 252 |
| DSSRGDRSSV | 253 |
| SQSRGDLVPL | 254 |
| MPARGDLVQT | 255 |
| SGVRGDRLAV | 256 |
| SNERGDQHRL | 257 |
| STLRGDYAVS | 258 |
| SNARGDIMTT | 259 |
| TTGRGDQARL | 260 |
| TTLRGDHSQL | 261 |
| TNLRGDYQLT | 262 |
| STSRGDYVTH | 263 |
| SATRGDYSIL | 264 |
| QATRGDLQHT | 265 |
| HDDRGDRRML | 266 |
| SSHRGDLLAT | 267 |
| SYQRGDQHNM | 268 |
| SHQRGDYNTL | 269 |
| MDQRGDMSVV | 270 |
| QIARGDLHVP | 271 |
| TYTRGDQHMM | 272 |
| DASRGDRTSL | 273 |
| SDRRGDLHPQ | 274 |
| SATRGDHSTL | 275 |
| AYQRGDFTSV | 276 |
| TGNRGDLQSV | 277 |
| AWRRGDLENA | 278 |
| NAARGDHGLL | 279 |
| SYSRGDHQAL | 280 |
| DGVRGDRTVV | 281 |
| SSERGDKMST | 282 |
| TSMRGDYTHV | 283 |
| SGVRGDRIII | 284 |
| ENRRGDAQLL | 285 |
| TVYRGDREQH | 286 |
| LDRRGDLLNS | 287 |
| ESVRGDLRST | 288 |
| DKYRGDQRGL | 289 |
| TDRRGDAHIL | 290 |
| SQTRGDTSLM | 291 |
| DATRGDTRML | 292 |
| SMQRGDLTHA | 293 |
| SGTRGDTLIL | 294 |
| ANTRGDHQNF | 295 |
| TSQRGDHTQL | 296 |
| SHWRGDTSSL | 297 |
| ISGRGDRQEL | 298 |
| SVLRGDIGTI | 299 |
| QDSRGDLSGK | 300 |
| KPIRGDGAEL | 301 |
| TYMRGDLQHT | 302 |
| TELRGDLSST | 303 |
| SAMRGDHAVL | 304 |
| SWVRGDASTL | 305 |
| QYARGDPHAL | 306 |
| STNRGDIVVH | 307 |
| VNIRGDLHQT | 308 |
| REYRGDSSIL | 309 |
| STLRGDGSMM | 310 |
| SSQRGDITAH | 311 |
| TEFRGDRSTV | 312 |
| TSTRGDYVTV | 313 |
| NHLRGDVSAI | 314 |
| LERRGDLVST | 315 |
| NEYRGDHSHP | 316 |
| NWQRGDTSTL | 317 |
| TDSRGDRTVL | 318 |
| YAVRGDQHTI | 319 |
| FGVRGDTRDL | 320 |
| SSARGDTGML | 321 |
| SAMRGDQMGQ | 322 |
| SHTRGDLVTT | 323 |
| VTTRGDTAIL | 324 |
| EPTRGDRHSI | 325 |
| GSQRGDRLVL | 326 |
| QHYRGDLSTT | 327 |
| METRGDRQQL | 328 |
| EALRGDKVVL | 329 |
| NAVRGDQSYV | 330 |
| SPLRGDNRAL | 331 |
| ENRRGDTHAL | 332 |
| NSQRGDLTGT | 333 |
| DNSRGDHSRL | 334 |
| NTHRGDYASI | 335 |
| HTTRGDQQPL | 336 |
| EWRRGDQTAL | 337 |
| TAVRGDQTTV | 338 |
| EYLRGDRISQ | 339 |
| SRYRGDQETI | 340 |
| ASLRGDTVTL | 341 |
| VGVRGDSHVL | 342 |
| TNSRGDHTSL | 343 |
| VDQRGDQRAL | 344 |
| ASVRGDRLDV | 345 |
| TSYRGDLSSN | 346 |
| TSDRGDSSNR | 347 |
| SYMRGDLDMR | 348 |
| TDVRGDRSMI | 349 |
| SYVRGDSSTV | 350 |
| QVYRGDRESV | 351 |
| TDRRGDAVSF | 352 |
| TNSRGDLHTT | 353 |
| EAIRGDFKTL | 354 |
| TGIRGDTAGL | 355 |
| TSSRGDVQSL | 356 |
| SSRRGDTNEL | 357 |
| SNARGDHTPL | 358 |
| SELRGDRSPY | 359 |
| VHARGDTSIM | 360 |
| SALRGDHGTL | 361 |
| QTGRGDIAGM | 362 |
| TVARGDQIRL | 363 |
| TNSRGDSVAL | 364 |
| QNARGDSAVI | 365 |
| HYLRGDTTTL | 366 |
| ANARGDLQNT | 367 |
| QNIRGDIRTV | 368 |
| AALRGDHGQM | 369 |
| TTDRGDLSAR | 370 |
| LNVRGDLVHT | 371 |
| YSTRGDHTVL | 372 |
| TNARGDLTVS | 373 |
| TNARGDYTAL | 374 |
| AEGRGDTRGS | 375 |
| TNIRGDQSSV | 376 |
| VDRRGDTMTM | 377 |
| QIGRGDVTPL | 378 |
| MQARGDLLGV | 379 |
| NTQRGDYASV | 380 |
| SDTRGDRITQ | 381 |
| SAIRGDYRDV | 382 |
| VGVRGDVHPL | 383 |
| TVLRGDAERL | 384 |
| ASTRGDLSGQ | 385 |
| DSRRGDGQNL | 386 |
| IDKRGDIQGL | 387 |
| TVTRGDLLVN | 388 |
| DSTRGDRLGL | 389 |
| AANRGDYASL | 390 |
| RDYRGDTSSL | 391 |
| SNIRGDASLH | 392 |
| NLQRGDRTDF | 393 |
| NASRGDHSSM | 394 |
| TSMRGDYTHV | 395 |
| TSRRGDLNDV | 396 |
| QSHRGDVQSL | 397 |
| STTRGDYQSV | 398 |
| YSTRGDHAML | 399 |
| TSARGDLIHT | 400 |
| ATARGDRESF | 401 |
| ASTRGDYSTM | 402 |
| SNNRGDVQRL | 403 |
| ERLRGDQHII | 404 |
| SSIRGDHATL | 405 |
| NPTRGDIATV | 406 |
| MSTRGDLSVN | 407 |
| MDRRGDITYA | 408 |
| NATRGDLMSA | 409 |
| SDHRGDRTYQ | 410 |
| NWVRGDLSTA | 411 |
| SSHRGDLSAT | 412 |
| NATRGDYREI | 413 |
| DTLRGDLQTA | 414 |
| IAVRGDLTPV | 415 |
| TLLRGDLAHS | 416 |
| MNTRGDHTVL | 417 |
| TGHRGDIAVY | 418 |
| TSNRGDQMSN | 419 |
| SATRGDHTAI | 420 |
| SSVRGDREHA | 421 |
| TNSRGDLVVH | 422 |
| ETRRGDLSSQ | 423 |
| TYARGDITLT | 424 |
| SASRGDTLHL | 425 |
| DNQRGDLSSR | 426 |
| TQSRGDLRDA | 427 |
| ENRRGDLTNA | 428 |
| TSIRGDISSV | 429 |
| ISARGDTMTL | 430 |
| SRVRGDQDHW | 431 |
| SGNRGDIASY | 432 |
| NEQRGDRYNH | 433 |
| EALRGDVRAL | 434 |
| VDRRGDYTQM | 435 |
| NNVRGDYTSV | 436 |
| QTYRGDASVL | 437 |
| SNYRGDHGSL | 438 |
| TSSRGDLREV | 439 |
| QYTRGDVSNL | 440 |
| SDLRGDVRER | 441 |
| QNARGDTTAL | 442 |
| NSLRGDRDTM | 443 |
| SNSRGDQAQV | 444 |
| EATRGDRSTL | 445 |
| VPQRGDITNL | 446 |
| DKIRGDLRSI | 447 |
| SAARGDYSIV | 448 |
| VGLRGDLIGQ | 449 |
| QAGRGDLTVI | 450 |
| SKTRGDFQEA | 451 |
| GTARGDYSML | 452 |
| ISTRGDLVTI | 453 |
| TGTRGDREYM | 454 |
| MTTRGDLSIT | 455 |
| STSRGDMTSV | 456 |
| GPARGDFTSS | 457 |
| VDRRGDLGHV | 458 |
| NSGRGDMSQL | 459 |
| SQGRGDLERY | 460 |
| VFGRGDQYTL | 461 |
| IVGRGDLHLQ | 462 |
| SYSRGDTSRL | 463 |
| MSSRGDRATI | 464 |
| MSSRGDLTRV | 465 |
| QHTRGDFGNL | 466 |
| ETRRGDLSSQ | 467 |
| TGTRGDLRAI | 468 |
| QMARGDLSTA | 469 |
| LVERGDRIMN | 470 |
| GLVRGDLNAR | 471 |
| NNPRGDLSSV | 472 |
| HEKRGDLSNS | 473 |
| SQARGDLHQT | 474 |
| FTGRGDLRTA | 475 |
| QSIRGDTGHL | 476 |
| SAGRGDMSVL | 477 |
| SERRGDEHKW | 478 |
| TTGRGDLVSL | 479 |
| LGLRGDQIGQ | 480 |
| VALRGDLTHQ | 481 |
| MIGRGDQAPL | 482 |
| FQARGDHSTL | 483 |
| NSVRGDHSSL | 484 |
| WPNRGDLQST | 485 |
| SASRGDYQTL | 486 |
| TMQRGDLHTI | 487 |
| TALRGDLAGT | 488 |
| VNARGDFSQT | 489 |
| GWIRGDRTSM | 490 |
| TFSRGDHTVL | 491 |
| SGARGDLVGV | 492 |
| NNVRGDYTSV | 493 |
| QTARGDHVVL | 494 |
| TLIRGDLTRS | 495 |
| VTTRGDRLEL | 496 |

TABLE 6
Top Ranking Cardiac Muscle Specific n-mer
inserts and/or RGD Motifs

| Variant | SEQ ID NO: |
|---|---|
| EARRGDLQSP | 497 |
| SSSRGDRLEL | 498 |
| SSLRGDKDHL | 499 |
| ESLRGDVGAR | 500 |
| TSSRGDVQSL | 501 |
| LQSRGDVERQ | 502 |
| IDRRGDGHSL | 503 |
| QATRGDHAVV | 504 |
| QQTRGDLLPT | 505 |
| INVRGDYSST | 506 |
| YAYRGDTHNL | 507 |
| KPTRGDTATT | 508 |
| NERRGDHLGL | 509 |
| DRGRGDSTQG | 510 |
| MPARGDLVQT | 511 |
| QASRGDLQAS | 512 |
| LALRGDGALT | 513 |
| MERRGDQTQV | 514 |
| ITLRGDRDFP | 515 |
| TAARGDYIGM | 516 |
| EARRGDGVNV | 517 |
| SSGRGDTAGV | 518 |
| AISRGDTESV | 519 |
| MLIRGDHSAA | 520 |
| MKGRGDITDP | 521 |
| RNVRGDSDTT | 522 |
| LPYRGDTAYA | 523 |
| SNSRGDAQSK | 524 |
| TNQRGDRTEM | 525 |
| TEKRGDISGT | 526 |
| GSYRGDERQL | 527 |
| AGFRGDANAL | 528 |
| QYRRGDASLI | 529 |
| MLGRGDTLAL | 530 |
| VINRGDTTYS | 531 |
| EQLRGDLSNR | 532 |
| SHQRGDNPLY | 533 |
| SSIRGDMVRI | 534 |
| VHVRGDMATT | 535 |
| KDRRGDQTEY | 536 |
| TKQRGDTENY | 537 |
| SGERGDSMGL | 538 |
| TLARGDPKDA | 539 |
| MSVRGDLSTS | 540 |
| GYQRGDAMKL | 541 |
| DSSRGDRLSV | 542 |
| VNKRGDANEL | 543 |
| NERRGDSLYM | 544 |
| CAARGDLTNS | 545 |
| TTSRGDQYEK | 546 |
| ERTRGDIATQ | 547 |
| AAMRGDLSTT | 548 |
| TALRGDYAST | 549 |
| ASTRGDMSSS | 550 |
| AVGRGDSLRL | 551 |
| TRERGDTGHP | 552 |
| KYSRGDLSGE | 553 |
| SEVRGDILRS | 554 |
| MNKRGDHETQ | 555 |
| ASSRGDYDRV | 556 |
| SQLRGDNASS | 557 |
| NATRGDSGVT | 558 |
| STTRGDRMET | 559 |
| ASSRGDTSYL | 560 |
| DRTRGDLQSS | 561 |
| SNTRGDVVVT | 562 |
| ENARGDRTAM | 563 |
| NTNRGDATLH | 564 |
| HPGRGDSPTS | 565 |
| TSHRGDTLTT | 566 |
| KAPRGDNLPT | 567 |
| NMVRGDRDVL | 568 |
| GTSRGDFANV | 569 |
| ENKRGDVQQM | 570 |
| TANRGDTSSL | 571 |
| QSYRGDMSAS | 572 |
| NSARGDRLEL | 573 |
| TSNRGDRETF | 574 |
| SGVRGDVSVH | 575 |
| VTSRGDTASL | 576 |
| TGARGDTTVV | 577 |
| LVLRGDVRVM | 578 |
| SSARGDISGI | 579 |
| SNGRGDLHGS | 580 |
| VTGRGDTQYL | 581 |
| VVNRGDRTFN | 582 |
| LDKRGDPQWQ | 583 |
| QYRRGDYELS | 584 |
| VLPRGDQIIA | 585 |
| TSTRGDAAIV | 586 |
| TVFRGDRGND | 587 |
| SDSRGDTRGL | 588 |
| KDRRGDQTEY | 589 |
| TQLRGDLHPQ | 590 |
| MNGRGDASHI | 591 |
| WQSRGDRTIS | 592 |
| QDSRGDLSGK | 593 |
| SSARGDRADA | 594 |
| SSTRGDLTVN | 595 |
| ISPRGDSGGI | 596 |
| ANLRGDLHTP | 597 |
| TSSRGDLHFT | 598 |
| AATRGDTQTH | 599 |
| RDARGDLANS | 600 |
| YPHRGDQIMV | 601 |
| AGQRGDNWAQ | 602 |
| SARRGDQLEM | 603 |
| STGRGDQKLL | 604 |
| SLGRGDVSMV | 605 |
| ANERGDVSVL | 606 |
| SSIRGDLKLA | 607 |
| YSLRGDQHSS | 608 |
| STVRGDRAIT | 609 |
| HNSRGDPLGQ | 610 |
| RETRGDSSSF | 611 |
| VDKRGDLVLA | 612 |
| CNSRGDTQFL | 613 |
| TTARGDASQT | 614 |
| KMDRGDMAVS | 615 |
| KPTRGDTEHL | 616 |
| QYARGDPHAL | 617 |
| SSARGDTVAF | 618 |
| SERRGDLAPN | 619 |
| KFLRGDAGSS | 620 |
| QFNRGDQQRL | 621 |
| KFVRGDTLLP | 622 |
| EQRRGDHSAL | 623 |
| ADLRGDLRQS | 624 |
| SRVRGDRLED | 625 |
| SMTRGDTLRL | 626 |
| MSNRGDLREI | 627 |
| MLHRGDRMAV | 628 |
| NLQRGDRIPL | 629 |
| SSTRGDRLIS | 630 |
| EERRGDTHRL | 631 |
| MDKRGDQTIH | 632 |
| STKRGDLLSG | 633 |
| LIDRGDRMER | 634 |
| GRVRGDIVVV | 635 |
| DKLRGDLRLQ | 636 |
| NPTRGDNIGY | 637 |
| ELRRGDSANT | 638 |
| TAHRGDTSSI | 639 |
| HSTRGDRHLD | 640 |
| SATRGDLLST | 641 |
| TSSRGDSHAL | 642 |
| DANRGDLRDK | 643 |
| IDRRGDVRDM | 644 |
| TGTRGDLSTT | 645 |
| SNSRGDVALV | 646 |
| SYTRGDQHGL | 647 |

TABLE 7
Top Ranking Cardiac Muscle Specific n-mer
inserts and/or RGD Motifs

| Variant | SEQ ID NO.: |
|---|---|
| EARRGDLQSP | 648 |
| SSSRGDRLEL | 649 |
| SSLRGDKDHL | 650 |
| ESLRGDVGAR | 651 |
| TSSRGDVQSL | 652 |
| LQSRGDVERQ | 653 |
| IDRRGDGHSL | 654 |
| QATRGDHAVV | 655 |
| QQTRGDLLPT | 656 |
| INVRGDYSST | 657 |
| YAYRGDTHNL | 658 |
| KPTRGDTATT | 659 |
| NERRGDHLGL | 660 |
| DRGRGDSTQG | 661 |
| MPARGDLVQT | 662 |
| QASRGDLQAS | 663 |
| LALRGDGALT | 664 |
| MERRGDQTQV | 665 |
| ITLRGDRDFP | 666 |
| TAARGDYIGM | 667 |
| EARRGDGVNV | 668 |
| SSGRGDTAGV | 669 |
| AISRGDTESV | 670 |
| MLIRGDHSAA | 671 |
| MKGRGDITDP | 672 |
| RNVRGDSDTT | 673 |
| LPYRGDTAYA | 674 |
| SNSRGDAQSK | 675 |
| TNQRGDRTEM | 676 |
| TEKRGDISGT | 677 |
| GSYRGDERQL | 678 |
| AGFRGDANAL | 679 |
| QYRRGDASLI | 680 |
| MLGRGDTLAL | 681 |
| VINRGDTTYS | 682 |
| EQLRGDLSNR | 683 |
| SHQRGDNPLY | 684 |
| SSIRGDMVRI | 685 |
| VHVRGDMATT | 686 |
| KDRRGDQTEY | 687 |
| TKQRGDTENY | 688 |
| SGERGDSMGL | 689 |
| TLARGDPKDA | 690 |
| MSVRGDLSTS | 691 |
| GYQRGDAMKL | 692 |
| DSSRGDRLSV | 693 |
| VNKRGDANEL | 694 |
| NERRGDSLYM | 695 |
| CAARGDLTNS | 696 |
| TTSRGDQYEK | 697 |
| ERTRGDIATQ | 698 |
| AAMRGDLSTT | 699 |
| TALRGDYAST | 700 |
| ASTRGDMSSS | 701 |
| AVGRGDSLRL | 702 |
| TRERGDTGHP | 703 |
| KYSRGDLSGE | 704 |
| SEVRGDILRS | 705 |
| MNKRGDHETQ | 706 |
| ASSRGDYDRV | 707 |
| SQLRGDNASS | 708 |
| NATRGDSGVT | 709 |
| STTRGDRMET | 710 |
| ASSRGDTSYL | 711 |
| DRTRGDLQSS | 712 |
| SNTRGDVVVT | 713 |
| ENARGDRTAM | 714 |
| NTNRGDATLH | 715 |
| HPGRGDSPTS | 716 |
| TSHRGDTLTT | 717 |
| KAPRGDNLPT | 718 |
| NMVRGDRDVL | 719 |
| GTSRGDFANV | 720 |
| ENKRGDVQQM | 721 |
| TANRGDTSSL | 722 |
| QSYRGDMSAS | 723 |
| NSARGDRLEL | 724 |
| TSNRGDRETF | 725 |
| SGVRGDVSVH | 726 |
| VTSRGDTASL | 727 |
| TGARGDTTVV | 728 |
| LVLRGDVRVM | 729 |
| SSARGDISGI | 730 |
| SNGRGDLHGS | 731 |
| VTGRGDTQYL | 732 |
| VVNRGDRTFN | 733 |
| LDKRGDPQWQ | 734 |
| QYRRGDYELS | 735 |
| VLPRGDQIIA | 736 |
| TSTRGDAAIV | 737 |
| TVFRGDRGND | 738 |
| SDSRGDTRGL | 739 |
| KDRRGDQTEY | 740 |
| TQLRGDLHPQ | 741 |
| MNGRGDASHI | 742 |
| WQSRGDRTIS | 743 |
| QDSRGDLSGK | 744 |
| SSARGDRADA | 745 |
| SSTRGDLTVN | 746 |
| ISPRGDSGGI | 747 |
| ANLRGDLHTP | 748 |
| TSSRGDLHFT | 749 |
| AATRGDTQTH | 750 |
| RDARGDLANS | 751 |
| YPHRGDQIMV | 752 |
| AGQRGDNWAQ | 753 |
| SARRGDQLEM | 754 |
| STGRGDQKLL | 755 |
| SLGRGDVSMV | 756 |
| ANERGDVSVL | 757 |
| SSIRGDLKLA | 758 |
| YSLRGDQHSS | 759 |
| STVRGDRAIT | 760 |
| HNSRGDPLGQ | 761 |
| RETRGDSSSF | 762 |
| VDKRGDLVLA | 763 |
| CNSRGDTQFL | 764 |
| TTARGDASQT | 765 |
| KMDRGDMAVS | 766 |
| KPTRGDTEHL | 767 |
| QYARGDPHAL | 768 |
| SSARGDTVAF | 769 |
| SERRGDLAPN | 770 |
| KFLRGDAGSS | 771 |
| QFNRGDQQRL | 772 |
| KFVRGDTLLP | 773 |
| EQRRGDHSAL | 774 |
| ADLRGDLRQS | 775 |
| SRVRGDRLED | 776 |
| SMTRGDTLRL | 777 |
| MSNRGDLREI | 778 |
| MLHRGDRMAV | 779 |
| NLQRGDRIPL | 780 |
| SSTRGDRLIS | 781 |
| EERRGDTHRL | 782 |
| MDKRGDQTIH | 783 |
| STKRGDLLSG | 784 |
| LIDRGDRMER | 785 |
| GRVRGDIVVV | 786 |
| DKLRGDLRLQ | 787 |
| NPTRGDNIGY | 788 |
| ELRRGDSANT | 789 |
| TAHRGDTSSI | 790 |
| HSTRGDRHLD | 791 |
| SATRGDLLST | 792 |
| TSSRGDSHAL | 793 |
| DANRGDLRDK | 794 |
| IDRRGDVRDM | 795 |
| TGTRGDLSTT | 796 |
| SNSRGDVALV | 797 |
| SYTRGDQHGL | 798 |

TABLE 8
Top Ranking n-mer inserts, P-motifs,
and/or RGD motifs having CNS
and Skeletal Muscle Specificity

| Variant | SEQ ID NO: |
|---|---|
| GVLPQQGTPR | 799 |
| SERPEQGTQR | 800 |
| MTVPNQGTNR | 801 |
| NNTPSQGTSR | 802 |
| SRDPPQGTTR | 803 |
| GTVPSQGTSR | 804 |
| SFLPNQGTAR | 805 |
| SGDPSQGTYR | 806 |
| SREPNQGTPR | 807 |
| NSEPYQGTQR | 808 |
| IEGPTQGTSR | 809 |
| SSLPGQGTMR | 810 |
| STAPVQGTNR | 811 |
| DYSPGQGTVR | 812 |
| GSVPAQGTTR | 813 |
| NTNPDQGTMR | 814 |
| DDSPRQGTMR | 815 |
| SGQPEQGTAR | 816 |
| ERTPVQGTDR | 817 |
| ASTPSQGTER | 818 |
| GAVPTQGTPR | 819 |
| AGTPVQGTSR | 820 |
| NVNPNQGTDR | 821 |
| AREPVQGTVR | 822 |
| SSVPKQGTER | 823 |
| ESRPNQGTDR | 824 |
| EGVPIQGTHR | 825 |
| SKDPVQGTVR | 826 |
| MEPPHQGTGR | 827 |
| AISPNQGTTR | 828 |
| TRDPGQGTNR | 829 |
| QASPVQGTTR | 830 |
| GAHPSQGTDR | 831 |
| GYQPAQGTGR | 832 |
| MDNPLQGTLR | 833 |
| MSVPPQGTGR | 834 |
| SATPPQGTNR | 835 |
| NNGPDQGTNR | 836 |
| TDGPGQGTSR | 837 |
| SGNPTQGTMR | 838 |
| MEGPSQGTMR | 839 |
| TADPYQGTGR | 840 |
| AMNPVQGTPR | 841 |
| ADSPQQGTLR | 842 |
| TQVPTQGTVR | 843 |
| RGDPIQGTLR | 844 |
| SGPPYQGTER | 845 |
| TSNPVQGTGR | 846 |
| IVSPVQGTTR | 847 |
| AGEPMQGTVR | 848 |
| SGEPNQGTLR | 849 |
| STTPLQGTTR | 850 |
| GDEPRQGTTR | 851 |
| EMGPRQGTDR | 852 |
| GTTPMQGTSR | 853 |
| GQKPDQGTVR | 854 |
| TAIPSQGTSR | 855 |
| SVLPSQGTQR | 856 |
| SYNPSQGTVR | 857 |
| DAKPSQGTSR | 858 |
| MMAPLQGTDR | 859 |
| AEHPVQGTMR | 860 |
| SASPVQGTSR | 861 |
| ETNPRQGTDR | 862 |
| ALLPTQGTAR | 863 |
| GEAPSQGTNR | 864 |
| SWEPRQGTDR | 865 |
| WVSPKQGTER | 866 |
| YDGPSQGTSR | 867 |
| GTQPTQGTSR | 868 |
| NEVPRQGTPR | 869 |
| GSTPMQGTAR | 870 |
| LPRPVQGTDR | 871 |
| HALPSQGTDR | 872 |
| SWDPSQGTIR | 873 |
| TQLPTQGTIR | 874 |
| AVYPEQGTSR | 875 |
| NETPTQGTTR | 876 |
| QTGPTQGTER | 877 |
| TQVPTQGTVR | 878 |
| TMGPNQGTTR | 879 |
| GTMPDQGTLR | 880 |
| SESPVQGTVR | 881 |
| TVTPVQGTSR | 882 |
| REYPIQGTER | 883 |
| WYEPKQGTAR | 884 |
| AENPROGTER | 885 |
| STSPIQGTTR | 886 |
| TMSPPQGTGR | 887 |
| MEHPYQGTGR | 888 |
| EAYPKQGTER | 889 |
| TTEPTQGTQR | 890 |
| EVGPTQGTTR | 891 |
| NSTPDQGTHR | 892 |
| DAQPKQGTGR | 893 |
| SGVPVQGTVR | 894 |
| NGEPLQGTMR | 895 |
| VSVPGQGTLR | 896 |
| EFAPSQGTAR | 897 |
| DGNPQQGTGR | 898 |
| QNMPLQGTGR | 899 |
| WLGPNQGTDR | 900 |
| SSMPVQGTDR | 901 |
| EGAPGQGTGR | 902 |
| MVNPIQGTGR | 903 |
| SDMPGQGTGR | 904 |
| GDDPRQGTVR | 905 |
| ATMPTQGTPR | 906 |
| GGDPGQGTVR | 907 |
| TGSPIQGTLR | 908 |
| AEGPNQGTLR | 909 |
| SDGPGQGTAR | 910 |
| QNGPDQGTNR | 911 |
| EMIPVQGTGR | 912 |
| VERPPQGTDR | 913 |
| GTSPGQGTTR | 914 |
| QTMPPQGTVR | 915 |
| ESYPTQGTAR | 916 |
| DAVPKQGTVR | 917 |
| AEMPEQGTRR | 918 |
| SKDPIQGTTR | 919 |
| TASPIQGTGR | 920 |
| AGTPEQGTLR | 921 |
| SHDPSQGTLR | 922 |
| QYLPTQGTDR | 923 |
| MLRPVQGTDR | 924 |
| QAGPTQGTYR | 925 |
| EHSPLQGTSR | 926 |
| SASPTQGTLR | 927 |
| DTKPSQGTGR | 928 |
| MVVPIQGTGR | 929 |
| GTVPPQGTSR | 930 |
| EGAPIQGTAR | 931 |
| STVPNQGTTR | 932 |
| EGNPSQGTYR | 933 |
| SEGPHQGTLR | 934 |
| LDRPNQGTER | 935 |
| SSIPVQGTVR | 936 |
| AEVPHQGTNR | 937 |
| AAAPGQGTTR | 938 |
| LANPVQGTAR | 939 |
| MPMPGQGTGR | 940 |
| MSYPHQGTER | 941 |
| EGSPVQGTSR | 942 |
| EALPRQGTDR | 943 |
| ESYPRQGTDR | 944 |
| NASPGQGTSR | 945 |
| ENSPSQGTFR | 946 |
| LVMRGDTHSS | 947 |
| TLLRGDHSLT | 948 |
| TSTRGDVENR | 949 |
| NVSRGDVTRN | 950 |
| KYERGDVIQL | 951 |
| LTVRGDQSFQ | 952 |
| SALRGDGPLP | 953 |
| NVIRGDNSTT | 954 |
| SSNRGDVLTS | 955 |
| MHNRGDNQYT | 956 |
| NTSRGDSSST | 957 |
| SNFRGDSSVT | 958 |
| ATNRGDGRNL | 959 |
| NHGRGDPATL | 960 |
| TRERGDGNQE | 961 |
| TSDRGDTKPL | 962 |
| LTERGDRDRA | 963 |
| ERSRGDSSTT | 964 |
| GYNRGDDPPS | 965 |
| QSTRGDVLTS | 966 |
| NTTRGDQNPI | 967 |
| TNGRGDLTGT | 968 |
| SNQRGDSTRL | 969 |
| YRARGDGAID | 970 |
| QTLRGDNTPT | 971 |
| ELGRGDQHAR | 972 |
| SSFRGDNSER | 973 |
| THVRGDIVHN | 974 |
| VWQRGDITMN | 975 |
| TANRGDNTVT | 976 |
| ASVRGDSAMT | 977 |
| ESLRGDISTT | 978 |
| TSSRGDSTSV | 979 |
| LPTRGDTATT | 980 |
| MRDRGDTALE | 981 |
| DKPRGDFGTS | 982 |
| SRLRGDELNS | 983 |
| TNLRGDGKHT | 984 |
| LQGRGDPQVA | 985 |
| SVPRGDSAGL | 986 |
| SELRGDYPVN | 987 |
| GGLRGDASNP | 988 |
| DLNRGDVQRP | 989 |
| EQQRGDKAIT | 990 |
| RTFRGDPNVQ | 991 |
| RATRGDSAET | 992 |
| SYYRGDPQST | 993 |
| RTVRGDVSNP | 994 |
| SNNRGDGSFT | 995 |
| TGGRGDAHEW | 996 |
| MQTRGDPKML | 997 |
| LGIRGDPHTT | 998 |
| QLGRGDTNER | 999 |
| AVIRGDNVML | 1000 |
| VDRRGDVSAE | 1001 |
| VTLRGDKNST | 1002 |
| SMLRGDTRIS | 1003 |
| WTHRGDPSER | 1004 |
| LAVRGDVSHG | 1005 |
| MWLRGDSTVS | 1006 |
| SVKRGDGEPF | 1007 |
| TGRRGDNLEL | 1008 |
| TYMRGDTNQQ | 1009 |
| SNVRGDTLTT | 1010 |
| TWTRGDDAKL | 1011 |
| MSIRGDSQAQ | 1012 |
| LFTRGDSMVT | 1013 |
| YKERGDPLAP | 1014 |
| TPVRGDVTPS | 1015 |
| EVVRGDPSNY | 1016 |
| DTKRGDVAPR | 1017 |
| ITYRGDNSTL | 1018 |
| LNGRGDNLYE | 1019 |
| CTERGDQVSN | 1020 |
| TMHRGDNNRV | 1021 |
| TRDRGDNLTE | 1022 |
| YVLRGDSSSH | 1023 |
| GPTRGDSVLT | 1024 |
| TWMRGDDTHS | 1025 |
| TGTRGDTDII | 1026 |
| TNARGDGFQG | 1027 |
| TTHRGDPLPA | 1028 |
| SLGRGDRSEG | 1029 |
| ELSRGDTVTV | 1030 |
| GTLRGDNSPL | 1031 |
| MALRGDGVSG | 1032 |
| NAYRGDKTQA | 1033 |
| GAARGDGAGA | 1034 |
| TSWRGDSRDL | 1035 |
| VTLRGDNSRE | 1036 |
| VTERGDDRAL | 1037 |
| TSTRGDTLMT | 1038 |
| TDARGDAGQA | 1039 |
| TRDRGDHAPT | 1040 |
| SGTRGDTVDA | 1041 |
| SGNRGDSAST | 1042 |
| SSMRGDDSHY | 1043 |
| EGARGDKQAT | 1044 |
| LSSRGDLQER | 1045 |
| QLERGDNSSS | 1046 |
| VVARGDVVAL | 1047 |
| RQTRGDTTSA | 1048 |
| TTSRGDTASG | 1049 |
| AQGRGDPPTI | 1050 |
| ETHRGDQHDP | 1051 |
| RTYRGDDSEI | 1052 |
| ESLRGDLRHS | 1053 |
| NKLRGDDMTN | 1054 |
| ESVRGDSMAH | 1055 |
| QDARGDMTRM | 1056 |
| QTVRGDIQVT | 1057 |
| TSRRGDMSKH | 1058 |
| TAARGDTEVR | 1059 |
| KSERGDLRNT | 1060 |
| THPRGDNHIL | 1061 |
| SGPRGDSNGV | 1062 |
| QMHRGDQGQV | 1063 |
| ERGRGDPATL | 1064 |
| DGVRGDDNKL | 1065 |
| QLERGDKITL | 1066 |
| DSYRGDRTNL | 1067 |
| HWMRGDDTET | 1068 |
| LPGRGDPSTY | 1069 |
| LAPRGDYTLT | 1070 |
| NQMRGDGTHP | 1071 |
| ATSRGDSLQM | 1072 |
| NRDRGDTTES | 1073 |
| IAVRGDQRLT | 1074 |

TABLE 9
Top Ranking n-mer inserts, P-moitfs, and/or RGD
Motifs Having CNS and Skeletal Muscle Specificity

| Motif Variant | SEQ ID NO: |
|---|---|
| SGDPSQGTYR | 1075 |
| SREPNQGTPR | 1076 |
| NVSPTQGTSR | 1077 |
| AAQPYQGTGR | 1078 |
| SRDPPQGTTR | 1079 |
| SSVPKQGTER | 1080 |
| QASPVQGTTR | 1081 |
| SERPEQGTQR | 1082 |
| MTVPNQGTNR | 1083 |
| SLNPQQGTTR | 1084 |
| AGTPSQGTLR | 1085 |
| MEPPHQGTGR | 1086 |
| TQVPTQGTVR | 1087 |
| MASPGQGTLR | 1088 |
| SSLPGQGTMR | 1089 |
| DAVPKQGTVR | 1090 |
| MVNPIQGTGR | 1091 |
| LPRPVQGTDR | 1092 |
| SESPYQGTLR | 1093 |
| ESRPNQGTDR | 1094 |
| SGTPIQGTLR | 1095 |
| TTSPTQGTSR | 1096 |
| MEGPSQGTMR | 1097 |
| NNTPSQGTSR | 1098 |
| AQEPWQGTGR | 1099 |
| QNMPLQGTGR | 1100 |
| AQSPPQGTQR | 1101 |
| NTNPDQGTMR | 1102 |
| ETNPRQGTDR | 1103 |
| EMGPRQGTDR | 1104 |
| DYSPGQGTVR | 1105 |
| MLTPHQGTPR | 1106 |
| AMNPVQGTPR | 1107 |
| SQMPNQGTAR | 1108 |
| VAHPSQGTER | 1109 |
| LANPVQGTAR | 1110 |
| AIDPKQGTFR | 1111 |
| EAYPRQGTDR | 1112 |
| SSSPDQGTTR | 1113 |
| EGVPIQGTHR | 1114 |
| TAIPSQGTSR | 1115 |
| NVNPNQGTDR | 1116 |
| GVTPAQGTAR | 1117 |
| TMSPPQGTGR | 1118 |
| GTVPSQGTSR | 1119 |
| SEMPSQGTSR | 1120 |
| TVTPVQGTSR | 1121 |
| GSTPSQGTMR | 1122 |
| TKEPGQGTAR | 1123 |
| AGTPVQGTSR | 1124 |
| SESPKQGTDR | 1125 |
| ADNPIQGTGR | 1126 |
| ERTPVQGTDR | 1127 |
| KHEPNQGTER | 1128 |
| TSTPTQGTPR | 1129 |
| LETPTQGTSR | 1130 |
| GATPNQGTAR | 1131 |
| FGSPAQGTGR | 1132 |
| ASNPMQGTTR | 1133 |
| SYSPLQGTTR | 1134 |
| SALPAQGTNR | 1135 |
| SQTPTQGTVR | 1136 |
| EGTPAQGTGR | 1137 |
| TDVPYQGTGR | 1138 |
| GHEPNQGTAR | 1139 |
| SSTPGQGTAR | 1140 |
| TGLPMQGTGR | 1141 |
| TQVPTQGTVR | 1142 |
| SDRPYQGTDR | 1143 |
| VSVPGQGTLR | 1144 |
| DSGPGQGTTR | 1145 |
| ATIPVQGTDR | 1146 |
| MTSPIQGTVR | 1147 |
| TRDPGQGTNR | 1148 |
| YPGPEQGTGR | 1149 |
| SNSPPQGTER | 1150 |
| GTSPIQGTLR | 1151 |
| MVSPTQGTSR | 1152 |
| MEGPHQGTGR | 1153 |
| IEGPTQGTSR | 1154 |
| SKEPVQGTSR | 1155 |
| TMGPNQGTTR | 1156 |
| QNYPTQGTTR | 1157 |
| SESPVQGTVR | 1158 |
| SYAPSQGTAR | 1159 |
| TSQPTQGTGR | 1160 |
| QIRPSQGTDR | 1161 |
| DGSPNQGTGR | 1162 |
| SGEPNQGTLR | 1163 |
| ASLPVQGTSR | 1164 |
| VVNPTQGTIR | 1165 |
| EDLPRQGTGR | 1166 |
| SFLPNQGTAR | 1167 |
| KGTPSQGTER | 1168 |
| SFTPVQGTSR | 1169 |
| GVLPQQGTPR | 1170 |
| NSPPPQGTNR | 1171 |
| AREPVQGTVR | 1172 |
| GTTPMQGTSR | 1173 |
| TLLRGDHSLT | 1174 |
| TSTRGDVENR | 1175 |
| NVSRGDVTRN | 1176 |
| KYERGDVIQL | 1177 |
| LTVRGDQSFQ | 1178 |
| SALRGDGPLP | 1179 |
| NVIRGDNSTT | 1180 |
| SSNRGDVLTS | 1181 |
| MHNRGDNQYT | 1182 |
| NTSRGDSSST | 1183 |
| SNFRGDSSVT | 1184 |
| ATNRGDGRNL | 1185 |
| NHGRGDPATL | 1186 |
| TRERGDGNQE | 1187 |
| TSDRGDTKPL | 1188 |
| LTERGDRDRA | 1189 |
| ERSRGDSSTT | 1190 |
| GYNRGDDPPS | 1191 |
| QSTRGDVLTS | 1192 |
| NTTRGDQNPI | 1193 |
| TNGRGDLTGT | 1194 |
| SNQRGDSTRL | 1195 |
| YRARGDGAID | 1196 |
| QTLRGDNTPT | 1197 |
| ELGRGDQHAR | 1198 |
| SSFRGDNSER | 1199 |
| THVRGDIVHN | 1200 |
| VWQRGDITMN | 1201 |
| TANRGDNTVT | 1202 |
| ASVRGDSAMT | 1203 |
| ESLRGDISTT | 1204 |
| TSSRGDSTSV | 1205 |
| LPTRGDTATT | 1206 |
| MRDRGDTALE | 1207 |
| DKPRGDFGTS | 1208 |
| SRLRGDELNS | 1209 |
| TNLRGDGKHT | 1210 |
| LQGRGDPQVA | 1211 |
| SVPRGDSAGL | 1212 |
| SELRGDYPVN | 1213 |
| GGLRGDASNP | 1214 |
| DLNRGDVQRP | 1215 |
| EQQRGDKAIT | 1216 |
| RTFRGDPNVQ | 1217 |
| RATRGDSAET | 1218 |
| SYYRGDPQST | 1219 |
| RTVRGDVSNP | 1220 |
| SNNRGDGSFT | 1221 |
| TGGRGDAHEW | 1222 |
| MQTRGDPKML | 1223 |
| LGIRGDPHTT | 1224 |
| QLGRGDTNER | 1225 |
| AVIRGDNVML | 1226 |
| VDRRGDVSAE | 1227 |
| VTLRGDKNST | 1228 |
| SMLRGDTRIS | 1229 |
| WTHRGDPSER | 1230 |
| LAVRGDVSHG | 1231 |
| MWLRGDSTVS | 1232 |
| SVKRGDGEPF | 1233 |
| TGRRGDNLEL | 1234 |
| TYMRGDTNQQ | 1235 |
| SNVRGDTLTT | 1236 |
| TWTRGDDAKL | 1237 |
| MSIRGDSQAQ | 1238 |
| LFTRGDSMVT | 1239 |
| YKERGDPLAP | 1240 |
| TPVRGDVTPS | 1241 |
| EVVRGDPSNY | 1242 |
| DTKRGDVAPR | 1243 |
| ITYRGDNSTL | 1244 |
| LNGRGDNLYE | 1245 |
| CTERGDQVSN | 1246 |
| TMHRGDNNRV | 1247 |
| TRDRGDNLTE | 1248 |
| YVLRGDSSSH | 1249 |
| GPTRGDSVLT | 1250 |
| TWMRGDDTHS | 1251 |
| TGTRGDTDII | 1252 |
| TNARGDGFQG | 1253 |
| TTHRGDPLPA | 1254 |
| SLGRGDRSEG | 1255 |
| ELSRGDTVTV | 1256 |
| GTLRGDNSPL | 1257 |
| MALRGDGVSG | 1258 |
| NAYRGDKTQA | 1259 |
| GAARGDGAGA | 1260 |
| TSWRGDSRDL | 1261 |
| VTLRGDNSRE | 1262 |
| VTERGDDRAL | 1263 |
| TSTRGDTLMT | 1264 |
| TDARGDAGQA | 1265 |
| TRDRGDHAPT | 1266 |
| SGTRGDTVDA | 1267 |
| SGNRGDSAST | 1268 |
| SSMRGDDSHY | 1269 |
| EGARGDKQAT | 1270 |
| LSSRGDLQER | 1271 |
| QLERGDNSSS | 1272 |
| VVARGDVVAL | 1273 |
| RQTRGDTTSA | 1274 |
| TTSRGDTASG | 1275 |
| AQGRGDPPTI | 1276 |
| ETHRGDQHDP | 1277 |
| RTYRGDDSEI | 1278 |
| ESLRGDLRHS | 1279 |
| NKLRGDDMTN | 1280 |
| ESVRGDSMAH | 1281 |
| QDARGDMTRM | 1282 |
| QTVRGDIQVT | 1283 |
| TSRRGDMSKH | 1284 |
| TAARGDTEVR | 1285 |
| KSERGDLRNT | 1286 |
| THPRGDNHIL | 1287 |
| SGPRGDSNGV | 1288 |
| QMHRGDQGQV | 1289 |
| ERGRGDPATL | 1290 |
| DGVRGDDNKL | 1291 |
| QLERGDKITL | 1292 |
| DSYRGDRTNL | 1293 |
| HWMRGDDTET | 1294 |
| LPGRGDPSTY | 1295 |
| LAPRGDYTLT | 1296 |
| NOMRGDGTHP | 1297 |
| ATSRGDSLQM | 1298 |
| NRDRGDTTES | 1299 |
| IAVRGDQRLT | 1300 |

TABLE 10
Top Ranking n-mer inserts and/or P-Motifs
having CNS and Cardiac Muscle Specificity

| Variant | SEQ ID NO: |
|---|---|
| EGAPSQGTYR | 1301 |
| TASPSQGTTR | 1302 |
| GTEPKQGTLR | 1303 |
| NAGPSQGTSR | 1304 |
| MATPTQGTSR | 1305 |
| NGTPEQGTTR | 1306 |
| GTVPYQGTGR | 1307 |
| GGNPVQGTAR | 1308 |
| NSGPGQGTMR | 1309 |
| AHAPLOGTAR | 1310 |
| GVLPGQGTGR | 1311 |
| MMHPVQGTER | 1312 |
| MNGPQQGTLR | 1313 |
| TMLPVQGTGR | 1314 |
| LEHPTQGTGR | 1315 |
| TYNPTQGTVR | 1316 |
| GSLPSQGTIR | 1317 |
| EATPSQGTMR | 1318 |
| CANPVQGTNR | 1319 |
| SSGPNQGTVR | 1320 |
| GQKPDQGTVR | 1321 |
| MQVPTQGTSR | 1322 |
| GSLPIQGTSR | 1323 |
| WLGPGQGTER | 1324 |
| EQNPKQGTER | 1325 |
| DGSPVQGTGR | 1326 |
| SMHPTQGTAR | 1327 |
| SGQPWQGTDR | 1328 |
| EPGPPQGTNR | 1329 |
| SMDPAQGTQR | 1330 |
| GTNPGQGTIR | 1331 |
| TNVPSQGTMR | 1332 |
| IEKPSQGTER | 1333 |
| YEGPSQGTGR | 1334 |
| TPYPNQGTER | 1335 |
| TREPFQGTGR | 1336 |
| GTVPTQGTGR | 1337 |
| GSDPSQGTMR | 1338 |
| SFQPPQGTNR | 1339 |
| YATPSQGTVR | 1340 |
| GTGPHQGTLR | 1341 |
| ATQPDQGTGR | 1342 |
| NGFPIQGTER | 1343 |
| LGSPSQGTTR | 1344 |
| TEMPVQGTVR | 1345 |
| SNSPPQGTER | 1346 |
| GTMPQQGTGR | 1347 |
| MEHPTQGTGR | 1348 |
| MSGPSQGTNR | 1349 |
| SMLPHQGTSR | 1350 |
| VRDPNQGTSR | 1351 |
| LQAPSQGTAR | 1352 |
| GMEPPQGTVR | 1353 |
| VTTPLQGTDR | 1354 |
| MAAPVQGTPR | 1355 |
| STEPDQGTKR | 1356 |
| VGTPEQGTAR | 1357 |
| AGSPTQGTLR | 1358 |
| GNTPSQGTVR | 1359 |
| GGAPVQGTSR | 1360 |
| QGLPTQGTPR | 1361 |
| TTSPSQGTAR | 1362 |
| NHTPAQGTQR | 1363 |
| YTSPVQGTVR | 1364 |
| SGEPVQGTVR | 1365 |
| EGNPAQGTVR | 1366 |
| SSEPNQGTPR | 1367 |
| GAIPNQGTTR | 1368 |
| MAHPVQGTER | 1369 |
| VLHPVQGTDR | 1370 |
| AQDPSQGTSR | 1371 |
| LVDPIQGTIR | 1372 |
| ARDPMQGTGR | 1373 |
| SVTPVQGTLR | 1374 |
| AVTPAQGTLR | 1375 |
| MANPSQGTVR | 1376 |
| GSIPSQGTSR | 1377 |
| MGVPEQGTTR | 1378 |
| EGSPYQGTDR | 1379 |
| MGYPMQGTGR | 1380 |
| SWTPNQGTNR | 1381 |
| GNGPSQGTTR | 1382 |
| SEVPIQGTVR | 1383 |
| GQLPSQGTGR | 1384 |
| GHNPDQGTVR | 1385 |
| ETGPPQGTMR | 1386 |
| SALPDQGTAR | 1387 |
| MSSPPQGTAR | 1388 |
| SISPTQGTGR | 1389 |
| VSAPSQGTQR | 1390 |
| QMMPSQGTVR | 1391 |
| LASPPQGTSR | 1392 |
| SSDPKQGTPR | 1393 |
| ESEPRQGTTR | 1394 |
| NAGPDQGTVR | 1395 |
| MASPIQGTTR | 1396 |
| GPLPVQGTMR | 1397 |
| NSLPKQGTDR | 1398 |
| GSMPAQGTNR | 1399 |
| SAGPLQGTAR | 1400 |
| SGPPEQGTNR | 1401 |
| GTSPIQGTLR | 1402 |
| AMQPVQGTAR | 1403 |
| YTSPIQGTNR | 1404 |
| ANSPVQGTVR | 1405 |
| SPVPKQGTER | 1406 |
| AESPVQGTGR | 1407 |
| SMHPTQGTAR | 1408 |
| MSGPEQGTLR | 1409 |
| AQAPEQGTIR | 1410 |
| RLQPVQGTDR | 1411 |
| TSVPSQGTER | 1412 |
| AAVPVQGTPR | 1413 |
| GMAPGQGTAR | 1414 |
| GMMPAQGTNR | 1415 |
| SEHPSQGTMR | 1416 |
| SYAPSQGTAR | 1417 |
| SWSPIQGTSR | 1418 |
| SNTPDQGTSR | 1419 |
| GQQPAQGTQR | 1420 |
| NTIPGQGTSR | 1421 |
| SDRPSQGTDR | 1422 |
| SSAPIQGTTR | 1423 |
| RVEPSQGTER | 1424 |
| QHTPIQGTDR | 1425 |
| GTTPMQGTSR | 1426 |
| LEGPVQGTTR | 1427 |
| IGEPHQGTMR | 1428 |
| AGFPVQGTDR | 1429 |
| YSSPVQGTER | 1430 |
| MMTPGQGTPR | 1431 |
| SESPFQGTVR | 1432 |
| TVTPMQGTLR | 1433 |
| TEGPLQGTHR | 1434 |
| LRSPVQGTDR | 1435 |
| SVLPQQGTTR | 1436 |
| EEMPNQGTRR | 1437 |
| SRMPDQGTSR | 1438 |
| SISPLQGTTR | 1439 |
| GVHPDQGTGR | 1440 |
| GMMPVQGTGR | 1441 |
| MASPVQGTSR | 1442 |
| SPMPSQGTSR | 1443 |
| SAAPIQGTNR | 1444 |
| CDGPVQGTNR | 1445 |
| QHQPYQGTER | 1446 |
| YSYPTQGTTR | 1447 |
| IVGPSQGTAR | 1448 |
| TDKPVQGTDR | 1449 |
| GADPRQGTIR | 1450 |
| NTSPSQGTVR | 1451 |
| AGTPAQGTTR | 1452 |
| LRSPPQGTDR | 1453 |
| SDRPPQGTFR | 1454 |
| DAAPVQGTLR | 1455 |
| MGTPVQGTER | 1456 |
| NSCPGQGTCR | 1457 |
| VTSPAQGTNR | 1458 |
| SEQPSQGTQR | 1459 |
| SQAPPQGTLR | 1460 |
| ATTPGQGTGR | 1461 |
| LSGPGQGTYR | 1462 |
| LTVPEQGTVR | 1463 |
| EGKPEQGTYR | 1464 |
| SQNPNQGTDR | 1465 |
| SMIPGQGTVR | 1466 |
| ANAPNQGTMR | 1467 |
| QEGPLQGTQR | 1468 |
| MEPPLQGTTR | 1469 |
| ERAPYQGTDR | 1470 |
| LEGPTQGTAR | 1471 |
| LRQPDQGTTR | 1472 |
| DRMPGQGTSR | 1473 |
| EGGPVQGTQR | 1474 |
| EGLPIQGTTR | 1475 |
| SSTPVQGTVR | 1476 |
| VVGPTQGTER | 1477 |
| SVVPVQGTQR | 1478 |
| SLGPPQGTLR | 1479 |
| SFTPVQGTSR | 1480 |
| DGRPYQGTTR | 1481 |
| ASVPVQGTGR | 1482 |
| ATAPVQGTNR | 1483 |
| SAAPYQGTSR | 1484 |
| AGLPIQGTTR | 1485 |
| SKVPAQGTER | 1486 |
| ASAPMQGTMR | 1487 |
| SAYPTQGTMR | 1488 |
| VTGPDQGTGR | 1489 |
| MTQPGQGTIR | 1490 |
| ALGPSQGTSR | 1491 |
| GVIPVQGTSR | 1492 |
| CEVPSQGTAR | 1493 |
| SNAPAQGTVR | 1494 |
| SAQPNQGTPR | 1495 |
| ETGPAQGTAR | 1496 |
| GVSPAQGTVR | 1497 |

TABLE 11
Top Ranking n-mer inserts and/or P-Motifs
having Cardiac Muscle and CNS Specificity

| Variant | SEQ ID NO: |
|---|---|
| EGAPSQGTYR | 1498 |
| EATPSQGTMR | 1499 |
| GTEPKQGTLR | 1500 |
| MEHPTQGTGR | 1501 |
| SEMPSQGTSR | 1502 |
| GVLPGQGTGR | 1503 |
| GPLPVQGTMR | 1504 |
| SALPDQGTAR | 1505 |
| MATPTQGTSR | 1506 |
| NGTPEQGTTR | 1507 |
| GTVPYQGTGR | 1508 |
| GGNPVQGTAR | 1509 |
| FEAPSQGTGR | 1510 |
| AHAPLOGTAR | 1511 |
| MNGPQQGTLR | 1512 |
| MQGPTQGTDR | 1513 |
| TASPSQGTTR | 1514 |
| SESPSQGTSR | 1515 |
| MMHPVQGTER | 1516 |
| ETGPPQGTMR | 1517 |
| NHTPAQGTQR | 1518 |
| TNVPSQGTMR | 1519 |
| LEHPTQGTGR | 1520 |
| SSGPNQGTVR | 1521 |
| GQKPDQGTVR | 1522 |
| TMLPVQGTGR | 1523 |
| CANPVQGTNR | 1524 |
| GSDPSQGTMR | 1525 |
| DGSPVQGTGR | 1526 |
| TYNPTQGTVR | 1527 |
| WLGPGQGTER | 1528 |
| TPYPNQGTER | 1529 |
| SMHPTQGTAR | 1530 |
| STQPTQGTDR | 1531 |
| NRQPDQGTAR | 1532 |
| SESPVQGTVR | 1533 |
| FEVPQQGTLR | 1534 |
| IETPAQGTGR | 1535 |
| ISDPNQGTAR | 1536 |
| EFGPTQGTLR | 1537 |
| LDHPTQGTIR | 1538 |
| TDAPTQGTMR | 1539 |
| CEGPKQGTDR | 1540 |
| LGSPSQGTTR | 1541 |
| ATQPDQGTGR | 1542 |
| YATPSQGTVR | 1543 |
| MSGPSQGTNR | 1544 |
| GTMPQQGTGR | 1545 |
| MTSPDQGTMR | 1546 |
| AGSPTQGTLR | 1547 |
| ASDPKQGTYR | 1548 |
| GNTPSQGTVR | 1549 |
| GMEPPQGTVR | 1550 |
| GSPPEQGTGR | 1551 |
| LQAPSQGTAR | 1552 |
| LNGPVQGTTR | 1553 |
| YCTPQQGTTR | 1554 |
| HTDPTQGTGR | 1555 |
| SSYPAQGTAR | 1556 |
| SNSPPQGTER | 1557 |
| TEMPVQGTVR | 1558 |
| ARDPMQGTGR | 1559 |
| GGAPVQGTSR | 1560 |
| TTSPSQGTAR | 1561 |
| YTSPVQGTVR | 1562 |
| STEPDQGTKR | 1563 |
| EGNPAQGTVR | 1564 |
| MGVPEQGTTR | 1565 |
| EGSPYQGTDR | 1566 |
| LESPSQGTGR | 1567 |
| GQLPSQGTGR | 1568 |
| GHNPDQGTVR | 1569 |
| MAAPVQGTPR | 1570 |
| AVIPTQGTSR | 1571 |
| VTTPLQGTDR | 1572 |
| THLPGQGTGR | 1573 |
| QMMPSQGTVR | 1574 |
| TAWPTQGTTR | 1575 |
| ESEPRQGTTR | 1576 |
| NAGPDQGTVR | 1577 |
| TSYPVQGTLR | 1578 |
| MASPIQGTTR | 1579 |
| TNSPGQGTVR | 1580 |
| SGEPVQGTVR | 1581 |
| NSLPKQGTDR | 1582 |
| MANPSQGTVR | 1583 |
| AQAPLOGTPR | 1584 |
| GSMPAQGTNR | 1585 |
| SAGPLOGTAR | 1586 |
| SESPSQGTHR | 1587 |
| QGLPTQGTPR | 1588 |
| YTSPIQGTNR | 1589 |
| LVDPIQGTIR | 1590 |
| MGYPMQGTGR | 1591 |
| SPVPKQGTER | 1592 |
| SMHPTQGTAR | 1593 |
| DSKPPQGTSR | 1594 |
| TQMPDQGTHR | 1595 |
| AAVPVQGTPR | 1596 |
| GMMPAQGTNR | 1597 |
| SWSPIQGTSR | 1598 |
| INSPIQGTAR | 1599 |
| CSVPNQGTGR | 1600 |
| SATPTQGTFR | 1601 |
| SWTPNQGTNR | 1602 |
| SISPTQGTGR | 1603 |
| SSEPNQGTPR | 1604 |
| YSSPVQGTER | 1605 |
| LHTPSQGTPR | 1606 |
| SESPFQGTVR | 1607 |
| TDGPGQGTAR | 1608 |
| GAIPNQGTTR | 1609 |
| GNGPSQGTTR | 1610 |
| EEMPNQGTRR | 1611 |
| SRMPDQGTSR | 1612 |
| DSKPGQGTMR | 1613 |
| SISPLQGTTR | 1614 |
| SPMPSQGTSR | 1615 |
| NTSPSQGTVR | 1616 |
| SDRPPQGTFR | 1617 |
| AATPTQGTTR | 1618 |
| DAAPVQGTLR | 1619 |
| NSGPGQGTMR | 1620 |
| SPMPSQGTSR | 1621 |
| TSSPTQGTMR | 1622 |
| IKSPDQGTGR | 1623 |
| KSEPVQGTTR | 1624 |
| QYSPQQGTGR | 1625 |
| SQAPPQGTLR | 1626 |
| AGVPIQGTER | 1627 |
| GTSPIQGTLR | 1628 |
| GGTPAQGTQR | 1629 |
| MLMPSQGTDR | 1630 |
| VSAPSQGTQR | 1631 |
| LRTPVQGTDR | 1632 |
| RVEPSQGTER | 1633 |
| TSLPQQGTSR | 1634 |
| QEGPLQGTQR | 1635 |
| DAGPNQGTAR | 1636 |
| STNPTQGTVR | 1637 |
| MEPPLQGTTR | 1638 |
| NSVPGQGTLR | 1639 |
| GMAPGQGTAR | 1640 |
| LRQPDQGTTR | 1641 |
| TANPNQGTYR | 1642 |
| MAMPAQGTLR | 1643 |
| SNLPGQGTLR | 1644 |
| EGVPRQGTDR | 1645 |
| QVSPTQGTSR | 1646 |
| NAGPSQGTSR | 1647 |
| GTTPMQGTSR | 1648 |
| SAAPYQGTSR | 1649 |
| TVTPMQGTLR | 1650 |
| SEAPTQGTGR | 1651 |
| MMTPGQGTPR | 1652 |
| SVLPQQGTTR | 1653 |
| SGEPPQGTNR | 1654 |
| ALGPSQGTSR | 1655 |
| DGTPYQGTSR | 1656 |
| TEVPKQGTPR | 1657 |
| LPTPSQGTSR | 1658 |
| SVTPVQGTLR | 1659 |
| MYKPIQGTER | 1660 |
| MGTPVQGTER | 1661 |
| SDGPGQGTGR | 1662 |
| LQYPEQGTER | 1663 |
| AMQPVQGTAR | 1664 |
| MMNPGQGTLR | 1665 |
| SHLPNQGTVR | 1666 |
| ANSPVQGTVR | 1667 |
| GSAPDQGTLR | 1668 |
| VENPTQGTYR | 1669 |
| LGQPPQGTSR | 1670 |
| WAGPSQGTLR | 1671 |
| ANNPSQGTFR | 1672 |
| NQIPLQGTLR | 1673 |
| SNSPVQGTMR | 1674 |
| EVGPTQGTTR | 1675 |
| TVKPTQGTER | 1676 |
| MASPVQGTSR | 1677 |
| SWGPGQGTIR | 1678 |
| AETPNQGTFR | 1679 |
| GVHPDQGTGR | 1680 |
| GMMPVQGTGR | 1681 |
| AAVPLQGTTR | 1682 |
| SEVPIQGTVR | 1683 |
| MTLPGQGTPR | 1684 |
| SDRPDQGTAR | 1685 |
| MQVPTQGTSR | 1686 |
| MNGPLQGTSR | 1687 |
| NDKPVQGTAR | 1688 |
| LSNPVQGTGR | 1689 |
| SLTPVQGTQR | 1690 |
| AVTPVQGTSR | 1691 |
| AGFPVQGTDR | 1692 |
| AGTPAQGTTR | 1693 |
| RDSPGQGTER | 1694 |
| LDSPYQGTGR | 1695 |
| GSLPIQGTSR | 1696 |
| SGQPWQGTDR | 1697 |
| GQVPTQGTNR | 1698 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising:
a targeting moiety effective to target a muscle cell or both a muscle cell and a central nervous system (CNS) cell, wherein the targeting moiety comprises one or more n-mer inserts each comprising:
one or more P-motifs, wherein at least one P-motif comprises or consists of the amino acid sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7; or
one or more RGD motifs, wherein at least one of the RGD motifs comprises or consists of XₘRGDXₙ, wherein m is 0-4 amino acids, wherein n is 0-15 amino acids, and wherein Xₘ, and Xₙ are each independently selected from any amino acid; or
both, and
optionally a cargo, wherein the cargo is coupled to or is otherwise associated with the targeting moiety.

2. The composition of claim 1, wherein the one or more of the one or more P-motifs and one or more of the RGD motifs are each independently selected from any one set forth in one or more of SEQ ID NOs: 4-1698 (Tables 4-11).

3. The composition of any one of the preceding claims, wherein the one or more RGD motifs and/or the one or more P motifs are selected from any one or more set forth in:
a. SEQ ID NOs: 4-250 (Table 4);
b. SEQ ID NOs: 497-647 (Table 6);
c. SEQ ID NOs: 799-1074 (Table 8);
d. SEQ ID NOs: 1301-1497 (Table 10); or
e. any combination thereof.

4. The composition of any one of the preceding claims, wherein the one or more RGD motifs and/or the one or more P motifs are selected from any one or more set forth in:
a. SEQ ID NOs: 251-496 (Table 5);
b. SEQ ID NOs: 648-798 (Table 7);
c. SEQ ID NOs: 1498-1698 (Table 9);
d. SEQ ID NOs: 1075-1300 (Table 11); or
e. any combination thereof.

5. The composition of any one of the preceding claims, wherein the one or more RGD motifs and/or the one or more P motifs are selected from any one or more set forth in:
a. SEQ ID NOs: 4-250 (Table 4) and/or SEQ ID NOs: 251-496 (Table 5);
b. SEQ ID NOs: 497-647 (Table 6) and/or SEQ ID NOs: 648-798 (Table 7);
c. SEQ ID NOs: 799-1074 (Table 8) and/or SEQ ID NOs: 1498-1698 (Table 9); or
d. SEQ ID NOs: 1301-1497 (Table 10) and/or SEQ ID NOs: 1075-1300 (Table 11).

6. The composition of any one of the preceding claims, wherein the targeting moiety is effective target
a. a skeletal muscle cell;
b. a cardiac muscle cell;
c. a skeletal muscle cell and a CNS cell; or
d. a cardiac muscle cell and a CNS cell.

7. The composition of any one of the preceding claims, wherein the one or more n-mer inserts are each 3-25 or 3-15 amino acids in length.

8. The composition of any one of the preceding claims, wherein
a. X₁ is S, T, or A,
b. X₂ is L, V, F, or I, or
c. both.

9. The composition of any one of the preceding claims, wherein the one or more RGD motifs and/or one or more P-motifs is/are immediately preceded by AQ or DG in the targeting moiety.

10. The composition of any one of the preceding claims, wherein the targeting moiety comprises a polypeptide, a polynucleotide, a lipid, a polymer, a sugar, or a combination thereof.

11. The composition of any one of the preceding claims, wherein the targeting moiety comprises a viral protein.

12. The composition of claim 11, wherein the viral protein is a capsid protein.

13. The composition of any one of claims 11-12, wherein one or more of the n-mer inserts are incorporated into the viral protein such that at least one of the one or more RGD motifs, at least one of the one or more P motifs, or both is/are located between two amino acids of the viral protein such that at least one of the one or more RGD motifs and/or one or more P-motifs is external to a viral capsid.

14. The composition of any one of claims 11-13, wherein the viral protein is an adeno associated virus (AAV) protein.

15. The composition of claim 14, wherein the AAV protein is an AAV capsid protein.

16. The composition of any one of claims 14-15, wherein one or more of the one or more n-mer inserts are incorporated into the AAV protein such that at least one or more of the one more RGD motifs and/or at least one or more of the one or more P motifs are each inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

17. The composition of any one of claims 14-16, wherein at least one of the one or more n-mer inserts is incorporated into the AAV protein such that at least one of the one more RGD motifs and/or at least one of the one or more P motifs is inserted between amino acids 588 and 589 in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

18. The composition of any one of claims 14-17, wherein the AAV capsid protein is an engineered AAV capsid protein having reduced or eliminated uptake in a non-CNS and/or non-muscle cell as compared to a corresponding wild-type AAV capsid polypeptide.

19. The composition of claim 18, wherein the non-CNS and/or non-muscle cell is a liver cell.

20. The composition of any one of claims 18-19, wherein the wild-type capsid polypeptide is an AAV1, AAV2, AAV3, AAV4, AAV5, AAV6, AAV7, AAV8, AAV9, AAV rh.74, or AAV rh.10 capsid polypeptide.

21. The composition of any one of claims 18-20, wherein the engineered AAV capsid protein comprises one or more mutations that result in reduced or eliminated uptake in a non-CNS and/or non-muscle cell.

22. The composition of claim 21, wherein the one or more mutations are
a. in position 267,
b. in position 269,
c. in position 504,
d. in position 505,
e. in position 590,
f. or any combination thereof
in the AAV9 capsid protein (SEQ ID NO: 1) or in one or more positions corresponding thereto in a non-AAV9 capsid polypeptide.

23. The composition of claim 22, wherein the non-AAV9 capsid protein is an AAV1, AAV2, AAV3, AAV4, AAV5, AAV6, AAV7, AAV8, AAV rh.74, or AAV rh.10 capsid polypeptide.

24. The composition of any one of claims 22-23, wherein the mutation in position 267 in the AAV9 capsid protein (SEQ ID NO: 1) or position corresponding thereto in a non-AAV9 capsid polypeptide is a G or X mutation to A, wherein X is any amino acid.

25. The composition of any one of claims 22-24, wherein the mutation in position 269 in the AAV9 capsid protein (SEQ ID NO: 1) or position corresponding thereto in a non-AAV9 capsid polypeptide is an S or X to T mutation, wherein X is any amino acid.

26. The composition of any one of claims 22-25, wherein the mutation in position 504 in the AAV9 capsid protein (SEQ ID NO: 1) or position corresponding thereto in a non-AAV9 capsid polypeptide is a G or X to A mutation, wherein X is any amino acid.

27. The composition of any one of claims 22-26, wherein the mutation in position 505 in the AAV9 capsid protein (SEQ ID NO: 1) or position corresponding thereto in a non-AAV9 capsid polypeptide is a P or X to A mutation, wherein X is any amino acid.

28. The composition of any one of claims 22-27, wherein the mutation in position 590 in the AAV9 capsid protein (SEQ ID NO: 1) or position corresponding thereto in a non-AAV9 capsid polypeptide is a Q or X to A mutation, wherein X is any amino acid.

29. The composition of any one of claims 21-23, wherein the engineered AAV capsid protein is an engineered AAV9 capsid polypeptide comprising a mutation at position 267, position 269 or both of a wild-type AAV9 capsid protein (SEQ ID NO: 1), wherein the mutation at position 267 is a G to A mutation and wherein the mutation at position 269 is an S to T mutation.

30. The composition of any one of claims 21-23, wherein the engineered AAV capsid protein is an engineered AAV9 capsid polypeptide comprising a mutation at position 590 of a wild-type AAV9 capsid protein (SEQ ID NO: 1), wherein the mutation at position 509 is a Q to A mutation.

31. The composition of any one of claims 21-23, wherein the engineered AAV capsid protein is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

32. The composition of any one of claims 1-31, wherein the composition is an engineered viral particle.

33. The composition of claim 32, wherein the engineered viral particle is an engineered AAV viral particle.

34. The composition of claim 33, wherein the AAV viral particle is an engineered AAV1, AAV2, AAV3, AAV4, AAV5, AAV6, AAV7, AAV8, AAV9, AAV rh.74, or AAV rh.10 viral particle.

35. The composition of any of claims 1-34, wherein the optional cargo is capable of treating or preventing a CNS, a muscle disease or disorder, or both.

36. The composition of any one of claims 1-35, wherein the muscle disease and/or CNS disease or disorder is an auto immune disease; a cancer; a muscular dystrophy; a neuro-muscular disease; a sugar or glycogen storage disease; an expanded repeat disease; a dominant negative disease; a cardiomyopathy; a viral disease; a progeroid disease; or any combination thereof.

37. The composition of any one of claims 1-36, wherein the optional cargo is a morpholino, a peptide-linked morpholino, an antisense oligonucleotide, a PMO, a therapeutic transgene, a polynucleotide encoding a therapeutic polypeptide or peptide, a PPMO, one or more peptides, one or more polynucleotides encoding a CRISPR-Cas protein, a guide RNA, or both, a ribonucleoprotein, wherein the ribonucleoprotein comprises a CRISPR-Cas system molecule, a therapeutic transgene RNA, or other gene modifying or therapeutic RNA and/or protein, or any combination thereof.

38. The composition of any one of claims 1-37, wherein the optional cargo is capable of inducing exon skipping in a gene, optionally a dystrophin gene.

39. The composition of any one of claims 1-38, wherein the cargo is a mini- or micro-dystrophin gene.

40. The composition of claim 39, wherein the mini- or micro-dystrophin gene comprises spectrin-like repeats 1, 2, 3, and 24, and optionally an nNOS domain.

41. The composition of any one of claims 35-40, wherein the expanded repeat disease is Huntington's disease, a Myotonic Dystrophy, or Facioscapulohumeral muscular dystrophy (FSHD).

42. The composition of any one of claims 35-41, wherein the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD.

43. The composition of claim 42, wherein the myotonic dystrophy is Type 1 or Type 2.

44. The composition of any one of claims 36-43, wherein the cardiomyopathy is dilated cardiomyopathy, hypertrophic cardiomyopathy, DMD-associated cardiomyopathy, or Dannon disease.

45. The composition of any one of claims 36-44, wherein the sugar or glycogen storage disease is a MPS type III disease or Pompe disease.

46. The composition of claim 45, wherein the MPS type III disease, is MPS Type IIIA, IIIB, IIIC, or HID.

47. The composition of any one of claims 36-46, wherein the neuro-muscular disease is Charcot-Marie-Tooth disease or Friedreich's Ataxia.

48. A vector system comprising:
a vector comprising:
one or more polynucleotides, wherein at least one of the one or more polynucleotides encodes all or part of a targeting moiety effective to target a muscle cell or both a muscle cell and a central nervous system (CNS) cell, wherein the targeting moiety comprises one or more n-mer inserts comprising:
one or more P-motifs, wherein at least one P-motif comprises or consists of the amino acid sequence XₘPX₁QGTX₂RXₙ (SEQ ID NO: 1699), wherein X₁, X₂, Xₘ, and Xₙ are each independently selected from any amino acid, wherein m is 0, 1, 2, or 3, and wherein n is 0, 1, 2, 3, 4, 5, 6, or 7; or
one or more RGD motifs, wherein at least one of the RGD motifs comprises or consists of XₘRGDXₙ, wherein m is 0-4 amino acids, wherein n is 0-15 amino acids, and wherein Xₘ, and Xₙ are each independently selected from any amino acid; or
both,
wherein at least one of the one or more polynucleotides encodes the at least one RGD motif, at least one P-motif, or both; and
optionally, a regulatory element operatively coupled to one or more of the one or more polynucleotides.

49. The vector system of claim 48, wherein the one or more of the one or more P-motifs and one or more of the RGD motifs are each independently selected from any one set forth in any one or more of SEQ ID NOs: 4-1698 (Tables 4-11).

50. The vector system of any one of the preceding claims, wherein the one or more RGD motifs and/or the one or more P motifs are selected from any one or more set forth in:
a. SEQ ID NOs: 4-250 (Table 4);
b. SEQ ID NOs: 497-647 (Table 6);
c. SEQ ID NOs: 799-1074 (Table 8);
d. SEQ ID NOs: 1301-1497 (Table 10); or
e. any combination thereof.

51. The vector system of any one of the preceding claims, wherein the one or more RGD motifs and/or the one or more P motifs are selected from any one or more set forth in:
a. SEQ ID NOs: 251-496 (Table 5);
b. SEQ ID NOs: 648-798 (Table 7);
c. SEQ ID NOs: 1498-1698 (Table 9);
d. SEQ ID NOs: 1075-1300 (Table 11); or
e. any combination thereof.

52. The vector system of any one of the preceding claims, wherein the one or more RGD motifs and/or the one or more P motifs are selected from any one or more set forth in:
a. SEQ ID NOs: 4-250 (Table 4) and/or SEQ ID NOs: 251-496 (Table 5);
b. SEQ ID NOs: 497-647 (Table 6) and/or SEQ ID NOs: 648-798 (Table 7);
c. SEQ ID NOs: 799-1074 (Table 8) and/or SEQ ID NOs: 1498-1698 (Table 9); or
d. SEQ ID NOs: 1301-1497 (Table 10) and/or SEQ ID NOs: 1075-1300 (Table 11).

53. The vector system of any one of the preceding claims, wherein the targeting moiety is effective target
a. a skeletal muscle cell;
b. a cardiac muscle cell;
c. a skeletal muscle cell and a CNS cell; or
d. a cardiac muscle cell and a CNS cell.

54. The vector system of any one of the preceding claims, wherein the one or more n-mer inserts are each 3-25 or 3-15 amino acids in length.

55. The vector system of any one of the preceding claims, wherein
a. X₁ is S, T, or A,
b. X₂ is L, V, F, or I, or
c. both.

56. The vector system of any one of the preceding claims, wherein the n-mer insert is immediately preceded by AQ or DG in the targeting moiety.

57. The vector system of any one of claims 48-56, further comprising a cargo.

58. The vector system of claim 57, wherein the cargo is a cargo polynucleotide and is optionally operatively coupled to one or more of the one or more polynucleotides encoding all or part of the targeting moiety.

59. The vector system of any one of claims 48-58, wherein the vector system is capable of producing virus particles, virus particles that contain the cargo, or both.

60. The vector system of any one of claims 48-59, wherein the vector system is capable of producing a polypeptide comprising one or more of the targeting moieties.

61. The vector system of claim 60, wherein the polypeptide is a viral polypeptide.

62. The vector system of claim 61, wherein the viral polypeptide is a capsid polypeptide.

63. The vector system of claim 62, wherein the capsid polypeptide is an adeno associated virus (AAV) capsid polypeptide.

64. The vector system of any one of claims 61-63, wherein the virus particles are AAV virus particles.

65. The vector system of any one of claims 51-52, wherein the AAV virus particles or AAV capsid polypeptide are engineered AAV1, AAV2, AAV3, AAV4, AAV5, AAV6, AAV7, AAV8, AAV9, AAV rh.74, or AAV rh.10 viral particles or polypeptides.

66. The vector system of any one of claims 61-63, wherein one or more of the one or more n-mer inserts are incorporated in the targeting moiety such that at least one of the one or more RGD motifs, at least one of the one or more P motifs, or both is/are located between two amino acids of the viral protein such that at least one of the one or more RGD motifs and/or at least one or more P-motifs is external to a viral capsid of the virus particles.

67. The vector system of any one of claims 63-66, wherein one or more of the one or more n-mer inserts are incorporated into the AAV protein such that at least one or more of the one more RGD motifs and/or at least one or more of the one or more P motifs are each inserted between any two contiguous amino acids independently selected from amino acids 262-269, 327-332, 382-386, 452-460, 488-505, 527-539, 545-558, 581-593, 598-599, 704-714, or any combination thereof in an AAV9 capsid polypeptide or in an analogous position in an AAV1, AAV2, AAV3, AAV4, AAV5, AAV6, AAV7, AAV8, AAV rh.74, AAV rh.10 capsid polypeptide.

68. The vector system of any one of claims 63-67, wherein at least one of the one or more n-mer inserts is incorporated into the AAV protein such that at least one of the one more RGD motifs and/or at least one of the one or more P motifs is inserted between amino acids 588 and 589 in the AAV9 capsid polynucleotide or in an analogous position in an AAV1, AAV2, AAV3, AAV4, AAV5, AAV6, AAV7, AAV8, AAV rh.74, AAV rh.10 capsid polypeptide.

69. The vector system any one of claims 63-68, wherein the AAV capsid protein is an engineered AAV capsid protein having reduced or eliminated uptake in a non-CNS cell or a non-muscle cell as compared to a corresponding wild-type AAV capsid polypeptide.

70. The vector system of claim 69, wherein the non-CNS or non-muscle cell is a liver cell.

71. The vector system of any one of claims 69-70, wherein the wild-type capsid polypeptide is an AAV1, AAV2, AAV3, AAV4, AAV5, AAV6, AAV7, AAV8, AAV9, AAV rh.74, or AAV rh.10 capsid polypeptide.

72. The vector system of any one of claims 69-70, wherein the engineered AAV capsid protein comprises one or more mutations that result in reduced or eliminated uptake in a non-CNS cell.

73. The vector system of claim 72, wherein the one or more mutations are
a. in position 267,
b. in position 269,
c. in position 504,
d. in position 505,
e. in position 590,
f. or any combination thereof
in the AAV9 capsid protein (SEQ ID NO: 1) or in one or more positions corresponding thereto in a non-AAV9 capsid polypeptide.

74. The vector system of claim 73, wherein the non-AAV9 capsid protein is an AAV1, AAV2, AAV3, AAV4, AAV5, AAV6, AAV7, AAV8, AAV rh.74, or AAV rh.10 capsid polypeptide.

75. The vector system of any one of claims 73-74, wherein the mutation in position 267 in the AAV9 capsid protein (SEQ ID NO: 1) or position corresponding thereto in a non-AAV9 capsid polypeptide is a G or X mutation to A, wherein X is any amino acid.

76. The vector system of any one of claims 73-75, wherein the mutation in position 269 in the AAV9 capsid protein (SEQ ID NO: 1) or position corresponding thereto in a non-AAV9 capsid polypeptide is an S or X to T mutation, wherein X is any amino acid.

77. The vector system of any one of claims 73-76, wherein the mutation in position 504 in the AAV9 capsid protein (SEQ ID NO: 1) or position corresponding thereto in a non-AAV9 capsid polypeptide is a G or X to A mutation, wherein X is any amino acid.

78. The vector system of any one of claims 73-77, wherein the mutation in position 505 in the AAV9 capsid protein (SEQ ID NO: 1) or position corresponding thereto in a non-AAV9 capsid polypeptide is a P or X to A mutation, wherein X is any amino acid.

79. The vector system of any one of claims 73-78, wherein the mutation in position 590 in the AAV9 capsid protein (SEQ ID NO: 1) or position corresponding thereto in a non-AAV9 capsid polypeptide is a Q or X to A mutation, wherein X is any amino acid.

80. The vector system of any one of claims 73-79, wherein the engineered AAV capsid protein is an engineered AAV9 capsid polypeptide comprising a mutation at position 267, position 269 or both of a wild-type AAV9 capsid protein (SEQ ID NO: 1), wherein the mutation at position 267 is a G to A mutation and wherein the mutation at position 269 is an S to T mutation.

81. The vector system of any one of claims 73-80, wherein the engineered AAV capsid protein is an engineered AAV9 capsid polypeptide comprising a mutation at position 590 of a wild-type AAV9 capsid protein (SEQ ID NO: 1), wherein the mutation at position 509 is a Q to A mutation.

82. The vector system of any one of claims 73-74, wherein the engineered AAV capsid protein is an engineered AAV9 capsid polypeptide comprising a mutation at position 504, position 505, or both of a wild-type AAV9 capsid protein (SEQ ID NO: 1), wherein the mutation at position 504 is a G to A mutation and wherein the mutation at position 505 is a P to A mutation.

83. The vector system of any one of claims 48-82, wherein the vector comprising the one or more polynucleotides does not comprise splice regulatory elements.

84. The vector system of any one of claims 48-83, further comprising a polynucleotide that encodes a viral rep protein.

85. The vector system of claim 84, wherein the viral rep protein is an AAV rep protein.

86. The vector system of any one of claims 84-85, wherein the polynucleotide that encodes the viral rep protein is on the same vector or a different vector as the one or more polynucleotides.

87. The vector system of any one of claims 84-86, wherein the polynucleotide that encodes the viral rep protein is operatively coupled to a regulatory element.

88. The vector system of any one of claims 48-87, wherein the vector system is capable of producing a composition or portion thereof as in any of claims 1-47.

89. A polypeptide encoded, produced, or both by a vector system as in any of claims 48-88.

90. The polypeptide of claim 89, wherein the polypeptide is a viral polypeptide.

91. The polypeptide of claim 90, wherein the viral polypeptide is an AAV polypeptide.

92. The polypeptide of any one of claims 89-91, wherein the polypeptide is coupled to or otherwise associated with a cargo.

93. A particle produced by a vector system as in any one of claims 48-88, optionally including a polypeptide as in any one of claims 89-92.

94. The particle of claim 93, wherein the particle is a viral particle.

95. The particle of claim 94, wherein the viral particle is an adeno-associated virus (AAV) particle, lentiviral particle, or a retroviral particle.

96. The particle of any one of claims 93-95, wherein the particle comprises a cargo.

97. The particle of any of claims 93-96, wherein the viral particle has a muscle tropism, or a muscle and central nervous system (CNS) tropism.

98. The vector system of any one of claims 48-88, the polypeptide as in any one of claims 89-92, or the particle of any one of claims 93-97, wherein the cargo is capable or preventing a CNS disease or, a muscle disease or disorder, or both a CNS and muscle disease or disorder.

99. The vector system of any one of claims 48-88 or 98, the polypeptide of any one of claims 89-92, or the particle of any one of claims 93-97, wherein the CNS or muscle disease or disorder is (a) an auto immune disease; (b) a cancer; (c) a muscular dystrophy; (d) a neuro-muscular disease; (e) a sugar or glycogen storage disease; (f) an expanded repeat disease; (g) a dominant negative disease; (h) a cardiomyopathy; (i) a viral disease; (j) a progeroid disease; or (k) any combination thereof.

100. The vector system, the polypeptide, or the particle of any one of claims 98-99, wherein the cargo is capable of inducing exon skipping in a gene.

101. The vector system, the polypeptide, or the particle of any one of claims 98-100, wherein the cargo is capable of inducing exon skipping in a dystrophin gene.

102. The vector system, the polypeptide, or the particle of any one of claims 98-101, wherein the cargo is a mini- or micro-dystrophin gene.

103. The vector system, the polypeptide, or the particle of claim 102, wherein the mini- or micro-dystrophin gene comprises spectrin-like repeats 1, 2, 3, and 24, and optionally an nNOS domain.

104. The vector system, the polypeptide, or the particle of any one of claims 98-103, wherein the expanded repeat disease is Huntington's disease, a Myotonic Dystrophy, or Facioscapulohumeral muscular dystrophy (FSHD).

105. The vector system, the polypeptide, or the particle of any one of claims 98-104, wherein the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD.

106. The vector system, the polypeptide, or the particle of claim 105, wherein the myotonic dystrophy is Type 1 or Type 2.

107. The vector system, the polypeptide, or the particle of any of claims 98-106, wherein the cardiomyopathy is dilated cardiomyopathy, hypertrophic cardiomyopathy, DMD-associated cardiomyopathy, or Dannon disease.

108. The vector system, the polypeptide, or the particle of any of claims 98-107, wherein the sugar or glycogen storage disease is a MPS type III disease or Pompe disease.

109. The vector system, the polypeptide, or the particle of claim 108, wherein the MPS type III disease, is MPS Type IIIA, IIIB, IIIC, or IIID.

110. The vector system, the polypeptide, or the particle of any of claims 98-109, wherein the neuro-muscular disease is Charcot-Marie-Tooth disease or Friedreich's Ataxia.

111. The polypeptide or the particle of any one of claims 89-110, wherein the polypeptide, the particle, or both have increased muscle cell potency, muscle cell specificity, reduced immunogenicity, or any combination thereof

112. A cell comprising:
a. a composition as in any of claims 1-47;
b. a vector system as in any one of claims 48-88 or 98-110;
c. a polypeptide as in any one of claims 89-92 or 98-111;
d. a particle of any one of claims 93-111; or
e. a combination thereof.

113. The cell of claim 87, wherein the cell is prokaryotic.

114. The cell of claim 87, wherein the cell is eukaryotic.

115. A pharmaceutical formulation comprising:
a. a composition as in any of claims 1-47;
b. a vector system as in any one of claims 48-88 or 98-110;
c. a polypeptide as in any one of claims 89-92 or 98-111;
d. a particle of any one of claims 93-111;
e. a cell as in any one of claims 99-101; or
f. a combination thereof; and
a pharmaceutically acceptable carrier.

116. A method of treating a muscle disease, disorder, or a symptom thereof, or both a muscle and a central nervous system disease, disorder, or a symptom thereof comprising:
administering, to the subject in need thereof,
a. a composition as in any of claims 1-47;
b. a vector system as in any one of claims 48-88 or 98-110;
c. a polypeptide as in any one of claims 89-92 or 98-111;
d. a particle of any one of claims 93-111;
e. a cell as in any one of claims 99-101;
f. a pharmaceutical formulation as in claim 102; or
g. a combination thereof.

117. The method of claim 116, wherein the central nervous system disease or disorder comprises a secondary muscle disease, disorder, or symptom thereof.

118. The method of any one of claims 116-117, wherein the central nervous system disease or disorder is Friedreich's Ataxia, Dravet Syndrome, Spinocerebellar Ataxia Type 3, Niemann Pick Type C, Huntington's Disease, Pompe Disease, Myotonic Dystrophy Type 1, Glut1 Deficiency Syndrome (De Vivo Syndrome), Tay-Sachs, Spinal Muscular Atrophy, Alzheimer's disease, Amyotrophic lateral sclerosis (ALS), Danon disease, Rett Syndrome, Angleman Syndrome, or a combination thereof.

119. The method of any one of claims 116-118, wherein the CNS or muscle disease or disorder is (a) an auto immune disease; (b) a cancer; (c) a muscular dystrophy; (d) a neuro-muscular disease; (e) a sugar or glycogen storage disease; (f) an expanded repeat disease; (g) a dominant negative disease; (h) a cardiomyopathy; (i) a viral disease; (j) a progeroid disease; or (k) any combination thereof.

120. The method of any one of claims 116-119, wherein the expanded repeat disease is Huntington's disease, a Myotonic Dystrophy, or Facioscapulohumeral muscular dystrophy (FSHD).

121. The method of any one of claims 116-120, wherein the muscular dystrophy is Duchene muscular dystrophy, Becker Muscular dystrophy, a Limb-Girdle muscular dystrophy, an Emery-Dreifuss muscular dystrophy, a myotonic dystrophy, or FSHD.

122. The method of claim 116, wherein the myotonic dystrophy is Type 1 or Type 2.

123. The method of any one of claims 116-122, wherein the cardiomyopathy is dilated cardiomyopathy, hypertrophic cardiomyopathy, DMD-associated cardiomyopathy, or Dannon disease.

124. The method of any one of claims 116-123, wherein the sugar or glycogen storage disease is a MPS type III disease or Pompe disease.

125. The method of claim 124, wherein the MPS type III disease, is MPS Type IIIA, IIIB, IIIC, or HID.

126. The method of any of claims 116-125, wherein the neuro-muscular disease is Charcot-Marie-Tooth disease or Friedreich's Ataxia.
